SEQUENCE LISTING (14 sequences)

SEQ ID NO: 1 - protein, 361 aa, Homo sapiens
MDPLGAAKPQWPWRRCLAALLFQLLVAVCFFSYLRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 2 - DNA, 1694 bp, Homo sapiens
gagaaagcaggcaaccaccatgtcatttgaaaacagtttcatcgggatataattcgcaacccatacagtgaatccatttaagatactctgacccatggatcccctgggtgcagccaagccacaatggccatggcgccgctgtctggccgcactgctatttcagctgctggtggctgtgtgtttcttctcctacctgcgtgtgtcccgagacgatgccactggatcccctagggctcccagtgggtcctcccgacaggacaccactcccacccgccccaccctcctgatcctgctatggacatggcctttccacatccctgtggctctgtcccgctgttcagagatggtgcccggcacagccgactgccacatcactgccgaccgcaaggtgtacccacaggcagacacggtcatcgtgcaccactgggatatcatgtccaaccctaagtcacgcctcccaccttccccgaggccgcaggggcagcgctggatctggttcaacttggagccaccccctaactgccagcacctggaagccctggacagatacttcaatctcaccatgtcctaccgcagcgactccgacatcttcacgccctacggctggctggagccgtggtccggccagcctgcccacccaccgctcaacctctcggccaagaccgagctggtggcctgggcggtgtccaactggaagccggactcagccagggtgcgctactaccagagcctgcaggctcatctcaaggtggacgtgtacggacgctcccacaagcccctgcccaaggggaccatgatggagacgctgtcccggtacaagttctacctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactggatttctgcaaggcctgctggaaactgcagcaggaatccaggtaccagacggtgcgcagcatagcggcttggttcacctgagaggccggcatggtgcctgggctgccgggaacctcatctgcctggggcctcacctgctggagtcctttgtggccaaccctctctcttacctgggacctcacacgctgggcttcacggctgccaggagcctctcccctccagaagacttgcctgctggggacctcgcctgctggggacctcgcctgttggggacctcacctgctggggacctcacctgctggggaccttggctgctggaggctgcacctactgaggatgtcggcggtcggggactttacctgctgggacctgctcccagagaccttgccacactgaatctcacctgctggggacctcaccctggagggccctgggccctggggaactggcttacttggggccccacccgggagtgatggttctggctgatttgtttgtgatgttgttagccgcctgtgaggggtgcagagagatcatcacggcacggtttccagatgtaatactgcaaggaaaaaaaaaaaaaaaaaaaaaaaaa

SEQ ID NO: 3 - protein, 530 aa, Homo sapiens
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 4 - DNA, 2287 bp, Homo sapiens
gcggcagctgctttagaaggtctcgagcctcctgtaccttcccagggatgaaccgggccttccctctggaaggcgagggttcgggccacagtgagcgagggccagggcggtgggcgcgcgcagagggaaaccggatcagttgagagagaatcaagagtagcggatgaggcgcttgtggggcgcggcccggaagccctcgggcgcgggctgggagaaggagtgggcggaggcgccgcaggaggctcccggggcctggtcgggccggctgggccccgggcgcagtggaagaaagggacgggcggtgcccggttgggcgtcctggccagctcaccttgccctggcggctcgccccgcccggcacttgggaggagcagggcagggcccgcggcctttgcattctgggaccgcccccttccattcccgggccagcggcgagcggcagcgacggctggagccgcagctacagcatgagagccggtgccgctcctccacgcctgcggacgcgtggcgagcggaggcagcgctgcctgttcgcgccatgggggcaccgtggggctcgccgacggcggcggcgggcgggcggcgcgggtggcgccgaggccgggggctgccatggaccgtctgtgtgctggcggccgccggcttgacgtgtacggcgctgatcacctacgcttgctgggggcagctgccgccgctgccctgggcgtcgccaaccccgtcgcgaccggtgggcgtgctgctgtggtgggagcccttcggggggcgcgatagcgccccgaggccgccccctgactgccggctgcgcttcaacatcagcggctgccgcctgctcaccgaccgcgcgtcctacggagaggctcaggccgtgcttttccaccaccgcgacctcgtgaaggggccccccgactggcccccgccctggggcatccaggcgcacactgccgaggaggtggatctgcgcgtgttggactacgaggaggcagcggcggcggcagaagccctggcgacctccagccccaggcccccgggccagcgctgggtttggatgaacttcgagtcgccctcgcactccccggggctgcgaagcctggcaagtaacctcttcaactggacgctctcctaccgggcggactcggacgtctttgtgccttatggctacctctaccccagaagccaccccggcgacccgccctcaggcctggccccgccactgtccaggaaacaggggctggtggcatgggtggtgagccactgggacgagcgccaggcccgggtccgctactaccaccaactgagccaacatgtgaccgtggacgtgttcggccggggcgggccggggcagccggtgcccgaaattgggctcctgcacacagtggcccgctacaagttctacctggctttcgagaactcgcagcacctggattatatcaccgagaagctctggcgcaacgcgttgctcgctggggcggtgccggtggtgctgggcccagaccgtgccaactacgagcgctttgtgccccgcggcgccttcatccacgtggacgacttcccaagtgcctcctccctggcctcgtacctgcttttcctcgaccgcaaccccgcggtctatcgccgctacttccactggcgccggagctacgctgtccacatcacctccttctgggacgagccttggtgccgggtgtgccaggctgtacagagggctggggaccggcccaagagcatacggaacttggccagctggttcgagcggtgaagccgcgctcccctggaagcgacccaggggaggccaagttgtcagctttttgatcctctactgtgcatctccttgactgccgcatcatgggagtaagttcttcaaacacccatttttgctctatgggaaaaaaacgatttaccaattaatattactcagcacagagatgggggcccggtttccatattttttgcacagctagcaattgggctccctttgctgctgatgggcatcattgtttaggggtgaaggagggggttcttcctcaccttgtaaccagtgcagaaatgaaatagcttagcggcaagaagccgttgaggcggtttcctgaatttccccatctgccacaggccatatttgtggcccgtgcagcttccaaatctcatacacaactgttcccgattcacgtttttctggaccaaggtgaagcaaatttgtggttgtagaaggagccttgttggtggagagtggaaggactgtggctgcaggtgggactttgttgtttggattcctcacagccttggctcctga

SEQ ID NO: 5 - protein, 405 aa, Homo sapiens
MGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 6 - DNA, 1215 bp, Homo sapiens
atgggggcaccgtggggctcgccgacggcggcggcgggcgggcggcgcgggtggcgccgaggccgggggctgccatggaccgtctgtgtgctggcggccgccggcttgacgtgtacggcgctgatcacctacgcttgctgggggcagctgccgccgctgccctgggcgtcgccaaccccgtcgcgaccggtgggcgtgctgctgtggtgggagcccttcggggggcgcgatagcgccccgaggccgccccctgactgccggctgcgcttcaacatcagcggctgccgcctgctcaccgaccgcgcgtcctacggagaggctcaggccgtgcttttccaccaccgcgacctcgtgaaggggccccccgactggcccccgccctggggcatccaggcgcacactgccgaggaggtggatctgcgcgtgttggactacgaggaggcagcggcggcggcagaagccctggcgacctccagccccaggcccccgggccagcgctgggtttggatgaacttcgagtcgccctcgcactccccggggctgcgaagcctggcaagtaacctcttcaactggacgctctcctaccgggcggactcggacgtctttgtgccttatggctacctctaccccagaagccaccccggcgacccgccctcaggcctggccccgccactgtccaggaaacaggggctggtggcatgggtggtgagccactgggacgagcgccaggcccgggtccgctactaccaccaactgagccaacatgtgaccgtggacgtgttcggccggggcgggccggggcagccggtgcccgaaattgggctcctgcacacagtggcccgctacaagttctacctggctttcgagaactcgcagcacctggattatatcaccgagaagctctggcgcaacgcgttgctcgctggggcggtgccggtggtgctgggcccagaccgtgccaactacgagcgctttgtgccccgcggcgccttcatccacgtggacgacttcccaagtgcctcctccctggcctcgtacctgcttttcctcgaccgcaaccccgcggtctatcgccgctacttccactggcgccggagctacgctgtccacatcacctccttctgggacgagccttggtgccgggtgtgccaggctgtacagagggctggggaccggcccaagagcatacggaacttggccagctggttcgagcgg

SEQ ID NO: 7 - protein, 374 aa, Homo sapiens
MDPLGPAKPQWLWRRCLAGLLFQLLVAVCFFSYLRVSRDDATGSPRPGLMAVEPVTGAPNGSRCQDSMATPAHPTLLILLWTWPFNTPVALPRCSEMVPGAADCNITADSSVYPQADAVIVHHWDIMYNPSANLPPPTRPQGQRWIWFSMESPSNCRHLEALDGYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQQESRYQTVRSIAAWFT

SEQ ID NO: 8 - DNA, 1487 bp, Homo sapiens
acagacatgagccacctcgcccagccttttcatggctgagtgatattccattgtgtggatggatcacactagctactctgacccatggatcccctgggcccagccaagccacagtggctgtggcgccgctgtctggccgggctgctgtttcagctgctggtggctgtgtgtttcttctcctacctgcgtgtgtcccgagacgatgccactggatcccctaggccagggcttatggcagtggaacctgtcaccggggctcccaatgggtcccgctgccaggacagcatggcgacccctgcccaccccaccctactgatcctgctgtggacgtggccttttaacacacccgtggctctgccccgctgctcagagatggtgcccggcgcggccgactgcaacatcactgccgactccagtgtgtacccacaggcagacgcggtcatcgtgcaccactgggatatcatgtacaaccccagtgccaacctcccgccccccaccaggccgcaggggcagcgctggatctggttcagcatggagtcccccagcaactgccggcacctggaagccctggacggatacttcaatctcaccatgtcctaccgcagcgactccgacatcttcacgccctatggctggctggagccgtggtccggccagcctgcccacccaccgctcaacctctcggccaagaccgagctggtggcctgggcggtgtccaactggaagccggactcggccagggtgcgctactaccagagcctgcaggctcatctcaaggtggacgtgtacggacgctcccacaagcccctgcccaaggggaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggatgacttccagagccccaaggacctggcccggtacctgcaggagctggacaaggaccacgcccgctacctgagctactttcgctggcgggagacgctgcggcctcgctccttcagctgggcactggctttctgcaaggcctgctggaagctgcagcaggaatccaggtaccagacggtgcgcagcatagcggcttggttcacctgagaggccggcatggggcctgggctgccaggacctcactttcccagggcctcacctacctagggtctcactagtcgggggatttacctacctggggcctcggctgcctggggtctcacctgcctggggcctcacctgctggagtctttggtggccaggcatgtgacttacctgggatttcacttgccgggcttcactgccaggagcctcccctgctggggaccttgccagctggggctggggatggtgcctactggggaccttgctttctggaggctgca

SEQ ID NO: 9 - protein, 359 aa, Homo sapiens [sequence omitted]

SEQ ID NO: 10 - DNA, 2875 bp, Homo sapiens
ggggagatcatttctctccattccaggaagtttgcatagctccctggacttctgctttgcactgccctgcaggagtgggtggggaaaggaagtggctttgaggcacacagaggggcttggtgaggccaccggaggaagcttctgccaccaatatgggacctgtgcccagcctaccagaagagagcatctgaaaacatgtatcgacatggtaacccctctgcttgaagcctcacatggctccctattgccttggtgctgaacaccctatggctgaccgtggcccagcctctgcaacagctctgcctcctctccagtggtgaagacccagcctgctgagactcctcctgcagttcctcaacatgcctgcatttctgctgcctcagggcctttgcgaaggttgttccttgtaactggaatgcccttccatcccttttttattcaaaaggctgcaattttaattgaagaaagttcccttccaaggttcatgagttgcctgacttgcccaccggtttcctgcaagatcccttggcctggcacttagtgctcaggaaatatttggtgatgggccaactgagtgagaaggtgggatctggtgggaaggaaagcggaaaggtagaaattctgctcacttcctcattcccacctcccaaggaacccctggtgtccctgtggaacccgctttgggaaccggtggttcaggtcagccttttcactttgtactcaaagccacatcgcattgaagccacaggtggggcaaggtcatgcatgacctgagtctccaaatcccttcaccctgtttggttctgcaacggggattaggggagccccacgatttgttttcaaaggatgtccgggctccaggacaggatgccctgggtcacctgatgacaggtgtggtggttggaaagggcctggtttcagctccgggtacacttcctccttccttctgctgcgtggtgtggcctcttccacgtcctcagaatccagctgttactccgtccgcggcctctcagctctagggccctctgcacactggcccccccagatactctgacccatggatcccctgggcccggccaagccacagtggtcgtggcgctgctgtctgaccacgctgctgtttcacctgctgatggctgtgtgtttcttctcctatctgcgtgtgtctcaagacgatcccactgtgtaccctaatgggtcccgcttcccagacagcacagggacccccgcccactccatccccctgatcctgctgtggacgtggccttttaacaaacccatagctctgccccgctgctcagagatggtgcctggcacggctgactgcaacatcactgccgaccgcaaggtgtatccacaggcagacgcggtcatcgtgcaccaccgagaggtcatgtacaaccccagtgcccagctcccacgctcctcgaggcggcaggggcagcgatggatctggttcagcatggagtccccaagccactgctggcagctgaaagccatggacggatacttcaatctcaccatgtcctaccgcagcgactccgacatcttcacgccctacggctggctggagccgtggtccggccagcctgcccacccaccgctcaacctctcggccaagaccgagctggtggcctgggcagtgtccaactgggggccaaactccgccagggtgcgctactaccagagcctgcaggcccatctcaaggtggacgtgtacggacgctcccacaagcccctgccccagggaaccatgatggagacgctgtcccggtacaagttctatctggccttcaagaactccttgcaccccgactacatcaccgagaagctgtggaggaacgccctggaggcctgggccgtgcccgtggtgctgggccccagcagaagcaactacgagaggttcctgccgcccgacgccttcatccacgtggacgacttccagagccccaaggacctggcccggtacctgcaggagctggacaaggaccacgcccgctacctgagctactttcgctggcgggagacgctgcggcctcgctccttcagctgggcactcgctttctgcaaggcctgctggaaactgcaggaggaatccaggtaccagacacgcggcatagcggcttggttcacctgagaggctggtgtggggcctgggctgccaggaacctcattttcctggggcctcacctgagtgggggcctcatctacctaaggactcgtttgcctgaagcttcacctgcctgaggactcacctgcctgggacggtcacctgttgcagcttcacctgcctggggattcacctacctgggtcctcactttcctggggcctcacctgctggagtcttcggtggccaggtatgtcccttacctgggatttcacatgctggcttccaggagcgtcccctgcggaagcctggcctgctggggatgtctcctggggactttgcctactggggacctcggctgttggggactttacctgctgggacctgctcccagagaccttccacactgaatctcacctgctaggagcctcacctgctggggacctcaccctggagggcactgggccctgggaactggcacccatggggccccacccatgagtgatggttctggctgatttgtttgtgatgttgttagccgcctgtgaggggtgcagagagataatcaccgcaccgtttccagatgtaatactgcaaagaaaaccgatgatgaggccgggtgcggtggctcacacctgtaatcccagcactttgggaggccgaggcaggcggatcacaaggtcaggagatcgagaccatcctggccaatatggtgaaacccgtctctactaaaaatacaaaaaaaaaaaaaaaaaaaaaaaaaaaaa

SEQ ID NO: 11 - protein, 342 aa, Homo sapiens
MNNAGHGPTRRLRGLGVLAGVALLAALWLLWLLGSAPRGTPAPQPTITILVWHWPFTDQPPELPSDTCTRYGIARCHLSANRSLLASADAVVFHHRELQTRRSHLPLAQRPRGQPWVWASMESPSHTHGLSHLRGIFNWVLSYRRDSDIFVPYGRLEPHWGPSPPLPAKSRVAAWVVSNFQERQLRARLYRQLAPHLRVDVFGRANGRPLCASCLVPTVAQYRFYLSFENSQHRDYITEKFWRNALVAGTVPVVLGPPRATYEAFVPADAFVHVDDFGSARELAAFLTGMNESRYQRFFAWRDRLRVRLFTDWRERFCAICDRYPHLPRSQVYEDLEGWFQA

SEQ ID NO: 12 - DNA, 1101 bp, Homo sapiens
gctgactgatcctgggagactgtggatgaataatgctgggcacggccccacccggaggctgcgaggcttgggggtcctggccggggtggctctgctcgctgccctctggctcctgtggctgctggggtcagcccctcggggtaccccggcaccccagcccacgatcaccatccttgtctggcactggcccttcactgaccagcccccagagctgcccagcgacacctgcacccgctacggcatcgcccgctgccacctgagtgccaaccgaagcctgctggccagcgccgacgccgtggtcttccaccaccgcgagctgcagacccggcggtcccacctgcccctggcccagcggccgcgagggcagccctgggtgtgggcctccatggagtctcctagccacacccacggcctcagccacctccgaggcatcttcaactgggtgctgagctaccggcgcgactcggacatctttgtgccctatggccgcctggagccccactgggggccctcgccaccgctgccagccaagagcagggtggccgcctgggtggtcagcaacttccaggagcggcagctgcgtgccaggctgtaccggcagctggcgcctcatctgcgggtggatgtctttggccgtgccaatggacggccactgtgcgccagctgcctggtgcccaccgtggcccagtaccgcttctacctgtcctttgagaactctcagcaccgcgactacattacggagaaattctggcgcaacgcactggtggctggcactgtgccagtggtgctggggcccccacgggccacctatgaggccttcgtgccggctgacgccttcgtgcatgtggatgactttggctcagcccgagagctggcggctttcctcactggcatgaatgagagccgataccaacgcttctttgcctggcgtgacaggctccgcgtgcgactgttcaccgactggcgggaacgtttctgtgccatctgtgaccgctacccacacctaccccgcagccaagtctatgaggaccttgagggttggtttcaggcctgagatccgctggccgggggaggtgggtgtgggtggaagggctgggtgtc

SEQ ID NO: 13 - protein, 359 aa, Homo sapiens
MTSTSKGILRPFLIVCIILGCFMACLLIYIKPTNSWIFSPMESASSVLKMKNFFSTKTDYFNETTILVWVWPFGQTFDLTSCQAMFNIQGCHLTTDRSLYNKSHAVLIHHRDISWDLTNLPQQARPPFQKWIWMNLESPTHTPQKSGIEHLFNLTLTYRRDSDIQVPYGFLTVSTNPFVFEVPSKEKLVCWVVSNWNPEHARVKYYNELSKSIEIHTYGQAFGEYVNDKNLIPTISACKFYLSFENSIHKDYITEKLYNAFLAGSVPVVLGPSRENYENYIPADSFIHVEDYNSPSELAKYLKEVDKNNKLYLSYFNWRKDFTVNLPRFWESHACLACDHVKRHQEYKSVGNLEKWFWN

SEQ ID NO: 14 - DNA, 2565 bp, Homo sapiens
ggcgaagccggagatgggcagagagcgcgcgcggcgcagcagctccagattcactgctctcccctgcagctccccgcgcccccgccgctgtcgctgcctcggtgtcccccagccccagtcgcgctcttaggacagcgccgccaccgccgcctggccctgcctgcctcctgcgccgcgcagccctcgcgagcgccccggaaggcgctttacccctaggaccgatttagaatgtaataactcaaggatttgataatacagtgaagtagtataacaactgtctacgtgcttcccatgatatgttctctatattgaaaaattatgacatcaacatccaaaggaattcttcgcccatttttgattgtctgcattatcctgggctgtttcatggcatgtcttctcatttacatcaaacctaccaacagctggatcttcagtccaatggaatcagccagctctgtgctgaaaatgaaaaacttcttttccaccaaaactgattattttaatgaaactactattctggtgtgggtgtggccatttgggcagacctttgaccttacatcctgccaagcaatgttcaacatccaaggatgccatctcacaacggaccgttcactgtacaacaaatcccatgcagttctgatccatcaccgagacatcagttgggatctgacaaatttacctcagcaagctaggccacccttccagaaatggatttggatgaatttggaatcaccaactcacactccccaaaagagtggcattgagcacttgtttaacctgactctgacttaccgccgtgattcagatatccaagtgccttatggcttcttgacggtaagcacaaatcccttcgtgtttgaagtgccaagcaaagagaaattggtgtgctgggttgtgagtaactggaaccctgagcatgccagagtcaagtattacaatgagctaagcaaaagcattgaaatccatacctacgggcaagcatttggagaatatgtcaatgataaaaatttgattcctaccatatctgcttgtaaattttatctttcctttgaaaattcaatccacaaggattacatcacggaaaagctatacaatgcttttctggctggctctgtacctgttgttctgggaccatctagggaaaactatgagaattatattccagcagattcattcattcatgtggaagattataactctcccagtgagctagcaaagtatctgaaggaagtcgacaaaaacaataagttataccttagttactttaactggaggaaggatttcactgtaaatcttccacgattttgggaatcacatgcatgtttggcttgcgatcatgtgaaaaggcatcaagaatataagtctgttggtaatttagagaaatggttttggaattaaaatttttcatcacttgcacacttgataaatattttgatgagatatcatccaagtattgaggataagaagagatgcaacatactacttttgtgtcacaatttatttttatcaccctctctagggtaacgtgtatattttggtggagatttttaaaagctcagcatgagcaatcattccattcggttttaaattatcctgtatatacctaattatgtgcactggagagtaatttattcttcattatcatttgtaaacattgctttttcacatttttgtagttgtccataatgtaagcttgtggtttgattattgtttccacactgatcagctgtttaatctatttgggaaatgaagatgcacatcttaaagtatgtaaaattttcactaagtattacaatgtctagttccaactttgcatactataacagaggaagaacatgttgcgattgaattctaacctctttgactcctaagatgaatgaagtgtataactgtctctatttgatctattttttttacctgtttatcacatttgtgaaggtgaaattattcatggagtgaataagaaagatatgaagcagaactgttctattcaggaagctattagacttctcatttattttcattaagctgatttgcagctacttattctcatggtcttaaattaaattattcaagtatttttaaatatccaatttgttgtgattttcagcacctgggaagtaatcccaataatactttagaaaatctaagacagttctttctgctactgatgacactcattgtcataataaaacaaataatttcctcaaataacaaagaaaaatgatacctataaatatatttataaatggtgtcatttatgaacaatgtttaattatgtatcaatttaagatttttttctgaagccctaatatttaaaatggccttattttaccatatggatataagatttggctcataatgatgagccctatcatttgatttgagttctatcatttaagagagcctaactaaaattatcatcaaggtattaaatataagacgttaaatataataaagtggggatatatagaaaacacacagtgttagcacagagtaagatctcaatgcacatttgttggatgaataaataaatgcaattgaattcccaaaaaaaaaaaaaaa

(19) United States
(12) Patent Application Publication
(10) Pub. No.: US 2021/0123024 A1
(43) Pub. Date: Apr. 29, 2021

(54) COMPOSITIONS AND METHODS FOR PRODUCTION OF EXOFUCOSYLATED CELLS FOR CLINICAL APPLICATIONS

(71) Applicant: The Brigham and Women's Hospital, Boston, MA (US)

(72) Inventor: Robert Sackstein, Pinecrest, FL (US)

(21) Appl. No.: 17/125,204

(22) Filed: Dec. 17, 2020

Related U.S. Application Data
(63) Continuation of application No. PCT/US19/37217, filed on Jun. 14, 2019.
(60) Provisional application No. 62/686,386, filed on Jun. 18, 2018.

(51) Int. Cl. C12N 5/0775; C12Q 1/48

(52) U.S. Cl. CPC C12N 5/0662; C12Q 1/48; C12N 2501/724; C12N 2500/84; G01N 2333/91091; G01N 2400/00; C12N 2500/34

(57) ABSTRACT

The present disclosure provides, inter alia, compositions and methods for detecting changes in level of expression of cell-surface Type 2 terminal lactosamines on a population of cultured cells propagated under different conditions. The disclosure also provides compositions and methods for enforcing stably expressed glycans on human cells. In certain embodiments, the compositions and/or methods utilize one or more members of the α(1,3)-fucosyltransferase family. In certain embodiments, glycoengineered CD44 glycosylated product (e.g. HCELL) is stable for at least 48 hours at 4° C., with retained expression after cell cryopreservation.

### CROSS-REFERENCE TO RELATED APPLICATIONS

The present application is a continuation of International Application No. PCT/US2019/037217, filed on Jun. 14, 2019, which claims benefit of U.S. Provisional Patent Application No. 62/686,386, filed on Jun. 18, 2018, which applications are incorporated by reference as if recited in full herein.

### GOVERNMENT FUNDING

This invention was made with government support under grants P01 HL107146 awarded by the National Institutes of Health. The government has certain rights in this invention.

### FIELD OF THE INVENTION

This disclosure relates to improved compositions and methods for enforcing expression of one or more glycans on a cell. In some embodiments, cells are cultured in a non-xenogeneic medium that promotes cell membrane expression of terminal lactosaminyl glycans, which can be further modified by glycosyltransferase-mediated addition of one or more monosaccharide constituents. In some embodiments, these glycans are present on the glycoprotein CD44 and are stably expressed for greater than 24 hours at 4° C. and after cryopreservation of the cells. This disclosure also relates to improved methods for analyzing the expression of a given glycan motif on a glycoconjugate via functional use of a glycosyltransferase and pertinent nucleotide sugar donor as a molecular probe, with detection of the product(s) of the glycosylation reaction thereby allowing for identification of the expression of the target acceptor glycan (i.e., the glycan motif).

### BACKGROUND OF THE INVENTION

### Modification of Glycans on a Cell Surface

In mammals, every cell of the body is covered with a sugar coat. Moreover, almost every protein and every lipid found on the cell surface, and those found within the extracellular fluids (e.g., blood), contains sugar modifications. These sugar decorations (which comprise a subset of “post-translational modifications”) impart key biologic effects on both the cell surface, and on each individual lipid (i.e., glycolipid) and protein (i.e., glycoprotein). Indeed, several key biologic effects are exclusively mediated by glycan determinants/composition. For example, the amount of sialic acid found on the surface of a blood leukocyte or a platelet dictates whether that cell will be destroyed (cleared) by the reticulo-endothelial system. Similarly, the sialic acid content of a glycoprotein dictates the half-life of that protein in circulation, with more sialylation generally yielding a longer half-life. Besides sialylation, the content and location of fucoses on the cell surface or on a particular glycoprotein or glycolipid imparts critical biology. For example, fucosylation of the Fc portion of antibodies dampens the ability of the antibody to participate in antibody-dependent cell-mediated cytotoxicity (ADCC). Thus, it is evident that the capacity to install a desired type or amount or a desired ratio of certain sugar structures can impart critical biologic effects on a cell, on a protein, or on a lipid. In many cases, the discrete compositional combination and relevant linkages (i.e., stereospecific localization) of certain monosaccharides (i.e, core sugar units) covalently clustered into oligosaccharides or polysaccharides impart a certain biologic property. To achieve the intended biologic effect, therefore, it is necessary to custom-modify the creation of that target oligosaccharide and/or polysaccharide motif on the surface of a given cell, on a given (glyco)protein, or on a given (glyco)lipid. As such, the ability to stereospecifically install a requisite quantity of one or more pertinent monosaccharides, or of an oligosaccharide/polysaccharide motif or a desired combination of various oligosaccharide/polysaccharide motifs on a given cell, glycolipid or glycoprotein, is highly desired.

Directing Migration of Blood-Borne Cells into Tissue

A key prerequisite to achieving the promise of all cell-based therapeutics (e.g., as in regenerative medicine, cell-based immunotherapy, etc.) is to deliver the relevant therapeutic cell(s) to affected sites of tissue injury/inflammation. Delivery of cells for clinical indications can be achieved by direct (local) injection into involved tissue(s), by intravascular administration (e.g., systemically or by catheter-based delivery to a particular vascular bed), or by application/placement of cells directly onto the affected area (e.g., for skin ulcers, burns, etc.). In all forms of cell administration, it would be advantageous for administered cells to possess membrane molecules that would promote lodgment of the cell within the administered site precisely within tissue microenvironments that are critical to achieve intended effect, e.g., control of inflammation, tissue repair, elimination of rejection, eradication of cancer, etc. One such microenvironmental site are the “perivascular areas” present in and around microvessels within an injured tissue, as it is well known that integrity of the microvasculature, and production of new microvessels (“angiogenesis”), is a critical prerequisite to tissue regeneration/repair. Indeed, at all sites of tissue injury, inflammation, and cancer, endothelial cells within the microvessels of affected tissue(s) display a characteristic set of adhesion molecules that serve a key role in recruitment of circulating (blood-borne) cells to the target site. These endothelial molecules are upregulated by inflammatory cytokines such as tumor necrosis factor (TNF) and interleukin 1 (IL-1), and, in humans include the molecule E-selectin, and, in mouse, the molecules E-selectin and P-selectin, which are lectins belonging to a family of adhesion molecules known as “selectins” (to be described in more detail below). In addition, leukocytes that have been recruited to any inflammatory site (including cancer) or to a site of tissue injury/damage display L-selectin, the “leukocyte” selectin, and, therefore, expression of ligands for L-selectin on administered cells would promote lodgment of such cells to regions of leukocyte infiltrates within the affected tissue(s).

At first glance, direct delivery might seem to be the most efficient approach to cell administration, especially considering that a concentrated bolus of cells could be applied to an affected area. However, there are situations where local injection may actually be counterproductive to intended therapeutic effects, and, moreover, local injection is practical for only certain anatomic locations: 1) By introducing pertinent cells in media suspension under hydrostatic pressure, the injection procedure could harm the delivered cells and, furthermore, could further compromise tissue integrity and disrupt incipient tissue repair and/or host defense processes, thereby exacerbating the inflammatory condition or counteracting appropriate immune reactions in situ; 2) By virtue of being an invasive method, the injection needle/device (and the suspension solution) could induce target tissue damage and/or instigate collateral tissue damage; 3) Direct injection is most feasible for organs/tissues with well-defined anatomic boundaries (e.g., the heart), and is impractical for tissues without extensive connective tissue support (e.g., the lung); 4) The injection procedure could be technologically demanding and labor-intensive, requiring use of sophisticated delivery systems with substantial imaging support, especially for relatively inaccessible and/or fragile organs/tissues (e.g., the central nervous system); 5) Most importantly, many degenerative and inflammatory conditions, infections, and cancers, are widely distributed and multifocal in nature (e.g., osteoporosis, inflammatory bowel disease, multiple sclerosis, bacterial/fungal/parasitic infections, hematologic malignancies (leukemias/lymphomas/multiple myeloma), multifocal/metastatic cancer, etc.), and thus direct injection is neither practical nor effective. Thus, though there are clinical conditions/situations in which local injection is feasible, the vascular route of administration is mandated for all generalized “systemic” disorders, as well as for any tissue with problematic access and/or anatomy not amenable to local injection (e.g., the pancreas in diabetes, the lung in chronic obstructive pulmonary disease). The capacity to administer cells repeatedly with minimal effort is another important practical advantage of systemic infusion. Therefore, creation of methodologies to optimize the expression/activity of molecular effectors directing both the adhesion/lodgment of directly injected cells within the inflammatory milieu and the physiologic migration of intravascularly administered cells to the affected site(s) is key to achieving the tremendous promise of all cell-based therapeutics.

The capacity to direct migration of blood-borne cells to a predetermined location (“homing”) has profound implications for a variety of physiologic and pathologic processes. Recruitment of circulating cells to a specific anatomic site is initiated by discrete adhesive interactions between cells in flow and vascular endothelium at the target tissue(s). The molecules that mediate these contacts are called “homing receptors,” and, as defined historically, these structures pilot tropism of cells in blood to the respective target tissue. Historically, three “tissue-specific homing receptors” were described: L-selectin for peripheral lymph nodes, α₄β₇ (LPAM-1) for intestines and gut-associated lymphoid tissue, and a specialized sialofucosylated glycoform of the molecule P-selectin Glycoprotein Ligand-1 (PSGL-1) known, specifically, as the “Cutaneous Lymphocyte Antigen” (CLA) that promotes cell migration to skin (63). Notably, apart from these tissues, it had been recognized for several decades that circulating cells, especially hematopoietic stem cells (HSCs), navigate effectively to bone marrow (66), and several studies pointed to a role for selectins, predominantly E-selectin binding to HSC E-selectin ligands, in mediating recruitment of HSCs to marrow.

Migration of cells from the vascular compartment into tissues is a cascade of events, the first step (Step 1) of which involves tethering/rolling interactions of the blood-borne cells to endothelial cells on post-capillary venules. This process is mediated principally by the selectin class of adhesion molecules, a family of three glycoproteins known as E-selectin (CD62E), P-selectin (CD62P) and L-selectin (CD62L) that function as calcium-dependent lectins (1,2). These decelerative adhesive interactions provide opportunity for cells to react to chemokines within the milieu, thereby triggering integrin activation (Step 2), resulting in firm adherence (Step 3), and then transendothelial migration (Step 4) (3,4). This “multi-step paradigm” holds that tissue-specific migration is regulated by a discrete combination of homing receptor and chemokine receptor expression on a given circulating cell, allowing for recognition of a pertinent “traffic signal” displayed by the relevant vascular adhesive ligands and chemokines expressed within target endothelium in an organ-specific manner. Following engagement of homing receptor(s) directing trafficking of cells to bone marrow, several lines of evidence indicate that one chemokine in particular, SDF-1 (CXCL12), plays an essential role in Step 2-mediated recruitment of cells to this site (66; 67; 68). However, expression of SDF-1 is not limited to the marrow, and this chemokine is typically expressed at all sites of tissue injury/inflammation (69).

The most efficient effectors of Step 1 rolling interactions are the selectins (E-, P- and L-selectin) and their ligands (65). As the name implies, selectins are lectins that bind to specialized carbohydrate determinants, consisting of sialofucosylations containing an α(2,3)-linked sialic acid substitution(s) and an α(1,3)-linked fucose modification(s) prototypically displayed on terminal lactosamines as the tetrasaccharide sialyl Lewis X (sLe^{x}; Neu5Acα2-3Galβ1-4[Fucα1-3]GlcNAcβ1-R)) (65; 70); the “core” lactosamine unit in sLe^{X} is known as a “type 2” lactosamine which is comprised of galactose attached to N-acetylglucosamine in [symbol] (1,4) linkage (Galβ(1-4)GlcNAcβ1-R). The sLe^{X} glycan (also known as “CD15s”) is recognized by a variety of monoclonal antibodies (mAbs), including the mAb known as “CSLEX-1” and another mAb known as “HECA452.” Compared to HECA452, the CSLEX-1 mAb has a more restricted specificity in that it recognizes only sLe^{X}, whereas mAb HECA452 recognizes both sLe^{X} and the isomeric sialofucosylated type 1 lactosaminyl glycan known as sialylated Lewis A (sLe^{A}) in which fucose is attached in α(1,4)-linkage to N-acetylglucosamine within a type 1 sialylated lactosamine backbone (i.e., Neu5Acα2-3Galβ1-3 [Fucα1-4]GlcNAcβ1-R).

E- and P-selectin are the ‘vascular selectins’ and are constitutively expressed in skin and bone marrow microvessels (5,6), whereas L-selectin is expressed on mature leukocytes and hematopoietic stem cells (HSC) (7). In humans, expression of E-selectin (but not P-selectin) is markedly increased on post-capillary venules in response to inflammatory cytokines such as TNF-α and IL-1 (8). Thus, apart from constitutive expression in endothelial beds of marrow and skin, microvessels at all sites of tissue injury/inflammation prominently express E-selectin.

As stated above, selectins can bind to the isomeric sialofucosylated lactosaminyl glycans sLe^{A} and sLe^{X}, but bind preferentially to sLe^{X} (9), and, moreover, E-selectin binds sLe^{X} with 5- and 10-fold higher affinity than that of L- and P-selectin, respectively (10). Among hematopoietic cells, three integral membrane glycoproteins carry sLe^{X} decorations: P-selectin glycoprotein ligand-1 (PSGL-1), CD43, and CD44 (11-13). PSGL-1 is the main P-selectin and L-selectin ligand in leukocytes (14), and, when extensively modified with sLe^{X} motifs, it can also bind E-selectin; the E-selectin-binding glycoform of PSGL-1 is known as cutaneous lymphocyte antigen (CLA). An sLe^{X}-modified glycovariant of CD43 (known as “CD43-E”) binds E-selectin, and, similarly, sLe^{X} decorations of CD44 create a specialized sialofucosylated CD44 glycoform known as Hematopoietic Cell E-/L-selectin Ligand (HCELL). HCELL is natively expressed on human hematopoietic stem/progenitor cells (HSPCs), and it is the most potent E-selectin ligand expressed on human cells (12,15). HCELL is operationally defined as CD44 that binds to E-selectin and L-selectin under shear conditions, and is identified by Western blot analysis of cell lysates as a CD44 glycoform reactive with E-selectin-Ig chimera (E-Ig) and with mAb HECA452.

The E-selectin ligands of human HSPCs are well-characterized, and include the highly sialofucosylated “CLA” glycoform of PSGL-1 (71; 69) and HCELL (72; 73). CD44 is a rather ubiquitous cell membrane protein, but the HCELL phenotype is found predominantly on human HSPCs. In contrast to HCELL's rather restricted distribution, CLA/PSGL-1 is widely expressed among hematopoietic progenitors and more mature myeloid and lymphoid cells within the marrow, as well as on circulating leukocytes (71; 69). In addition to CLA and HCELL, human leukocytes and HSPCs can also express the “CD43-E” glycoform of CD43 (74; 64), and, in mouse leukocytes, another E-selectin ligand known as E-selectin Ligand-1 (ESL-1) has been described (78). In all glycoprotein selectin ligands (e.g., CD43-E, CLA, and HCELL), binding to E-selectin (and, also, to L-selectin and P-selectin) is critically dependent on α(2,3)-sialic acid and α(1,3)-fucose modifications of terminal lactosaminyl glycans (72; 73; 75; 76). On human HSPCs, HCELL displays the pertinent sialofucosylated lactosamine selectin binding determinants on N-glycans (77; 75). In vitro assays of E- and L-selectin binding under hemodynamic shear stress indicate that HCELL is the most potent ligand for these molecules expressed on any human cell (72; 76). Importantly, though E-selectin is constitutively expressed on microvascular endothelium of the marrow and skin, this molecule is prominently expressed on endothelial beds at all sites of inflammation-both acute and chronic types-regardless of whether it is induced by direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, SIRS, etc.), neoplasia (e.g., breast cancer, lung cancer, lymphoma, etc.), immunologic/autoimmune conditions (e.g., graft vs. host disease, multiple sclerosis, diabetes, inflammatory bowel disease, lupus erythematosus, rheumatoid arthritis, psoriasis, etc.), degenerative diseases (e.g., osteoporosis, osteoarthritis, Alzheimer's disease, etc.), congenital/genetic diseases (e.g., muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). Indeed, E-selectin is expressed all over the endothelial cell, i.e., on both the luminal and abluminal sides of the endothelium.

### Glycosylation of Cells

Within cells, assembly of glycans on N-linked glycoproteins and on glycolipids is initiated in the ER, whereas O-glycosylation of proteins is initiated in the Golgi. In each case, glycan extension occurs by step-wise addition of monosaccharide units via the action of glycosyltransferases, type II integral membrane enzymes that stereo- and regiospecifically link the relevant monosaccharide to the pertinent substrate(s) (known as glycan “acceptors”). In humans, apart from sLe^{X}, the terminal (unsialylated) fucosylated lactosaminyl glycan known as Lewis-X (Le^{X}, also called “CD15”: Gal-β(1,4)-[Fuc-α(1,3)]-GlcNAc-R) bears major biological significance. These trisaccharide (Le^{X}) and tetrasaccharide (sLe^{X}) structures are displayed on the cell surface on both glycoproteins and glycolipids, and, in each case, the final step in their assembly requires α(1,3)-linked fucose modifications of N-acetylglucosamine (GlcNAc) within respective unsialylated or sialylated terminal Type 2 lactosamine (LacNAc) acceptors, i.e., Gal-β(1,4)-GlcNAc-R or NeuAc-α(2,3)-Gal-β(1,4)-GlcNAc-R. Importantly, sLe^{X} can only be created by fucosylation of sialylated LacNAc, as there is no mammalian sialyltransferase that can place sialic acid in α(2,3)-linkage to Gal in Le^{X} to create sLe^{X}. Thus, the biosynthesis of Le^{X} and sLe^{X} in each case critically pivots on fucose addition that is programmed by glycosyltransferases known as α(1,3)-fucosyltransferases (α(1,3)-FTs), which, in humans, constitute a family of six Golgi isoenzymes: FTIII (FT3), FTIV (FT4), FTV (FT5), FTVI (FT6), FTVII (FT7), and FTIX (FT9).

Display of Le^{X} and sLe^{X} are each very tightly regulated among mammalian cells (63), indicating that they each serve highly specialized biology. Le^{X} is well-known to mediate a variety of important cellular functions in development and immunity. In mice, Le^{X} is known as stage-specific embryonic antigen-1 (SSEA-1); it serves as a major marker of murine (but not human) embryonic stem cells (82, 83). In both mice and humans, Le^{X} is a marker for neural stem cells (85-88), and Le^{X}-bearing glycoconjugates mediate neural stem cell proliferation by activating the Notch signaling pathway (89). Importantly, Le^{X} is an immunomodulatory glycan motif, serving as one of the main glycans recognized by DC-SIGN (CD209), a C-type lectin (i.e., requiring Ca′ for ligand binding) expressed by dendritic cells (90); engagement of DC-SIGN polarizes dendritic cells toward a tolerogenic phenotype. Thus, in some applications it may be desirable to enforce Le^{X} expression on certain cell types, either alone or in variable combinations with enforced expression of E-selectin-binding glycan motifs (e.g., sLe^{X}). The [symbol] (1,3)-fucosyltransferases FT3, FT4, FTS, FT6, and FT9 can each create Le^{X} (i.e., can [symbol] (1,3)-fucosylate an unsialylated Type lactosamine), but FT9 is most potent in creating Le^{X} and this enzyme cannot make sLe^{X} (79).

The creation of sLe^{X} is engendered by [symbol] (1,3)-fucosylation of N-acetylglucosamines within sialylated Type 2 polylactosamines, a reaction that can be catalyzed by FT3, FT4 (modestly), FTS, FT6 and FT7 (with sLe^{X} most prominently created by FT6 and FT7) (79). Importantly, in addition to sLe^{X}, [symbol] (1,3)-fucosylation of N-acetylglucosamines within sialylated Type 2 polylactosamines can generate two other E-selectin-binding glycans known as s “VIM-2” (or “CD65s”) (in which fucose is [symbol] (1,3)-linked to GlcNAc within the penultimate LacNAc unit of a terminal polylactosaminyl glycan, i.e., NeuAc- [symbol] (2,3)-Gal- [symbol] (1,4)-GlcNAc- [symbol] (1,3)-Gal- [symbol] (1,4)-[Fuc- [symbol] (1,3)]-GlcNAc-R) and difucosyl sLe^{X} (in which fucose is [symbol] (1,3)-linked to GlcNAc within both the ultimate and penultimate LacNAc units, i.e., NeuAc- [symbol] (2,3)-Gal- [symbol] (1,4)-[Fuc- [symbol] (1,3)]-GlcNAc- [symbol] (1,3)-Gal- [symbol] (1,4)-[Fuc- [symbol] (1,3)]-GlcNAc-R). VIM-2 is predominantly created by FT3 and FTS, and difucosyl sLeX can be created by FT3, FTS, FT6 and FT7 (79).

### Mesenchymal Stem Cells

Human mesenchymal stem cells (hMSCs) are known to natively display sialylated terminal Type 2 lactosaminyl glycans (thus possessing glycosyltransferases necessary to create this structure), but are natively deficient in Le^{X} (CD15) and sLe^{X} CD15s) expression, and also lack expression of VIM-2 (CD65s) and difucosyl sLe^{X} (80, 81). hMSCs are multipotent cells with immunomodulatory and tissue reparative properties, and have garnered great interest in cell therapy for a variety of diseases (16-21). To date, hMSCs derived from bone marrow have been used most commonly in clinical trials (22,23). However, though these cells are frequently administered systemically, little attention has been paid to the fact that these cells ineffectively enter inflamed tissue(s): hMSCs natively lack expression of E-selectin ligands, and, consequently, have limited ability to engage vascular endothelium under hemodynamic shear conditions. Indeed, hMSCs do not express PSGL-1 nor CD43, but they characteristically express a sialylated Type 2 lactosamine-modified glycoform of CD44 (24). It is now clear that hMSCs derived from other tissue sources (e.g. adipose tissue) also have a glycosignature in which sialylated type 2 lactosamines are displayed on CD44 backbone (24). Treatment of hMSCs with α(1,3)-fucosyltransferases (e.g., FTVI or FTVII) in presence of the nucleotide sugar donor GDP-fucose converts CD44 into HCELL (24-25). This cell surface glycan engineering technology is called ‘glycosyltransferase programmed stereosubstitution’ (GPS). Following exofucosylation, HCELL expression lasts ˜24-48 h, gradually reversing to the endogenous CD44 phenotype by the normal protein turnover of the hMSCs surface. CD44 conversion to HCELL endows potent adhesion to E-selectin under fluid shear stress conditions, thus driving Step 1 interactions on E-selectin-bearing microvessels. Accordingly, GPS-mediated enforced HCELL expression enables homing of hMSCs to BM and skin and to all sites of inflammation/tissue injury, potentiating the use of these cells in cell therapy (26).

Apart from overcoming deficits in homing, successful application of hMSC-based therapies requires generating sufficient numbers of cells for pertinent indications. Standard ex vivo expansion media uses fetal bovine serum (FBS) as supplement. The use of FBS entails potential danger of infection with pathogenic adventitial agents (e.g., viruses and prions), and potential immunoreactivity via incorporation of xeno-epitopes on human cells (27-28). Ideally, a xenogeneic-free medium should be utilized (29), and human platelet lysate (HPL) represents an acceptable alternative to FBS (30,31). Most studies report an accelerated proliferation of hMSCs using HPL compared to FBS, without the associated chromosomal instability typically observed using FBS (32,33). Moreover, in xeno-infusions of hMSCs into mice, the risk of hMSC vascular complications such as pulmonary embolism appears to be lower in cells expanded using growth media supplements of human origin (34-36).

### Challenges to Identifying Cells Displaying Lactosaminyl Glycans

A critical barrier to increasing our understanding of the roles of glycans in human health and disease is the current requirement to utilize extremely specialized tools, expensive and requiring unique expertise, to unravel the composition and linkages of biologically-relevant glycans. There is a pressing need to develop innovative tools and methods to identify a family of biologically important cell surface glycan structures, such as the terminal lactosaminyl glycans, using human glycosyltransferase (GTs) as molecular probes, thereby displacing the need to perform technically-demanding, labor-intensive and costly mass spectrometry-based and nuclear magnetic resonance-based glycan analysis.

Mass spectrometry (MS) and nuclear magnetic resonance (NMR) can provide saccharide composition and linkage information on terminal lactosaminyl glycan structures. However, these are highly specialized and labor-intensive technologies, involving use of extremely expensive equipment that requires cumbersome and tedious workflows which are very costly and time-consuming. In addition, these approaches also require persons with unique skills for operating/maintaining sophisticated equipment and for analysis of the generated datasets, and, also typically require a significant amount of starting glycan material for scrutiny. Moreover, these techniques require isolation of glycoconjugates from the cell and subsequent release of glycans from scaffold proteins and lipids, and thus cannot readily track or yield information on the relative level of expression of relevant lactosaminyl glycoconjugates on native cell membranes nor of the topological display on the cell membrane itself. For these reasons, it would be highly advantageous to develop a method that can readily and accurately measure the expression of a given lactosaminyl glycan determinant displayed on a cell surface, and could readily and accurately evaluate differences in the expression of that determinant among two or more cell populations. Importantly, for both sialylated and unsialylated terminal lactosaminyl glycans (either Type 1 or Type 2), there are no antibodies or lectin probes that distinctly identify these structures, and, therefore, the only current methodology to detect such glycans is by using MS or NMR. These technological challenges have profoundly impeded the ability to assess cell culture conditions that can either increase or decrease expression of lactosaminyl glycans on a cell surface.

### SUMMARY OF THE INVENTION

The present disclosure provides methods for detecting differences in level of expression of Type 2 terminal lactosamines on a population of cultured cells propagated under different conditions. The present disclosure also provides compositions and methods for cell expansion and of exofucosylation of hMSCs cultured with HPL-supplemented medium, effective to safely produce HPL-expanded cells. In some embodiments, treatment of hMSCs with α(1,3)-fucosyltransferase VI (FTVI; which can modify unsialylated and sialylated lactosamines to create both Le^{X} and sLeX, respectively) or with fucosyltransferase VII (FTVII; which can only modify a sialylated lactosamine, thereby creating only sLeX) efficiently generated HCELL on hMSCs, which retained full cell viability and phenotype as determined by morphology, immunophenotypic profile and differentiation potential. In some embodiments, FTVI or FTVII is used to enforce hMSC sLeX expression for 48 h at 4° C., which persisted with cryopreservation of hMSCs. In some embodiments, exofucosylation is effective to leave karyotype unchanged, with no alteration in expression of c-Myc, in the gene expression profile (GEP), or in the receptor tyrosine kinase (RTK) phosphorylation profile of hMSCs. In some embodiments, the disclosure provides manufacturing protocols of both FTVI- and FTVII-treated hMSCs at clinical scale, fulfilling good manufacturing practice (GMP) standards, which is useful for enforcing HCELL expression on hMSCs for intended clinical indications.

According to some embodiments, the present disclosure provides a method of detecting a change of expression in cell-surface Type 2 terminal lactosamines on a population of cultured cells comprising the steps of: (a) contacting the cells with a glycosyltransferase and a donor nucleotide sugar, wherein the glycosyltransferase and donor nucleotide sugar are effective to enforce expression of a glycan; and (b) detecting the product glycan on the cells, wherein the contacting of step (a) is performed before and/or after any culture condition modification.

In some embodiments, the detecting of step (b) comprises an antibody-based technique that recognizes the product glycan. In some embodiments, the detecting of step (b) comprises a lectin-based technique that recognizes the product glycan. In some embodiments, the detecting of step (b) comprises a tag-based technique comprising a tag-modified sugar incorporated into the product glycan. In some embodiments, the antibody based technique is selected from the group consisting of enzyme-linked immunosorbent assay (ELISA), western blot, immunohistochemistry, immunocytochemistry, immunofluorescence, flow cytometry, immunoprecipitation, and combinations thereof.

In some embodiments, the glycosyltransferase is an α(1,3)-fucosyltransferase.

In some embodiments, the detecting of step (b) is effective to precisely identify the Type 2 terminal lactosamine target of an α(1,3)-fucosyltransferase by detecting one or more of product glycans consisting of sLe^{X}, Le^{X}, VIM-2, and Difucosyl sLe^{X}.

According to some embodiments, the present disclosure provides a method of detecting differences in level of expression of cell-surface Type 2 terminal lactosamines on a population of cultured cells propagated under different conditions comprising the steps of: (a) culturing a first population of cells under a first culture condition; (b) culturing a second population of cells under a second (different) culture condition; (c) contacting the first and second population of cells with a glycosyltransferase and a donor nucleotide sugar, wherein the glycosyltransferase and donor nucleotide sugar are effective to enforce expression of a glycan; and (d) detecting the glycan on the first and second population of cells.

In some embodiments, the glycosyltransferase is an α(1,3)-fucosyltransferase, and the detection step (d) is effective to precisely identify the Type 2 terminal lactosamine target of the fucosyltransferase by detecting one or more of glycans consisting of sLe^{X}, Le^{x}, VIM-2, and Difucosyl sLe^{X}.

In some embodiments, the detecting of step (d) comprises an antibody-based technique that recognizes the product glycan. In some embodiments, the detecting of step (b) comprises a lectin-based technique that recognizes the product glycan. In some embodiments, the detecting of step (b) comprises a tag-based technique comprising a tag-modified sugar incorporated into the product glycan. In some embodiments, the antibody based technique is selected from the group consisting of enzyme-linked immunosorbent assay (ELISA), western blot, immunohistochemistry, immunocytochemistry, immunofluorescence, flow cytometry, immunoprecipitation, and combinations thereof.

In some embodiments, the glycosyltransferase is an α(1,3)-fucosyltransferase.

In some embodiments, the first and second culture conditions comprise different supplements. In some embodiments, the first and second culture conditions comprise different feeder layers or matrix elements. In some embodiments, the first culture condition comprises fetal bovine serum (FBS) and the second culture condition comprises human platelet lysate (HPL).

In some embodiments, the first and second population of cells are stored at 4° C. or less for at least 24 hours after the contacting with the glycosyltransferase of step (c). In some embodiments, the first and second population of cells are frozen and then thawed after the contacting with the glycosyltransferase of step (c).

In some embodiments, the method further comprises the step of (e) detecting the viability of the cells.

In some embodiments, the cells are human mesenchymal stem cells (hMSCs).

In some embodiments, the first and or second culture condition comprises contacting the cells with a xenogeneic or non-xenogeneic protease effective to lift adherent cells from a culture plate. In some embodiments, the first and/or second culture condition comprises passaging the cells more than 2 times.

In some embodiments, the method further comprises the step of (e) selecting the culture condition that is effective to produce a desired amount of Type 2 lactosaminyl glycan. In some embodiments, the method further comprises the step of (e) selecting the culture condition that is effective to produce an increased amount of Type 2 lactosaminyl glycan.

The present disclosure also provides, an in vitro method for culture of cells comprising the steps of: (i) passaging the cells at least one time with a culture medium comprising a supplement wherein the cells comprise cell surface CD44 and the supplement is effective to maintain or increase the amount of a CD44 glycoform comprising sialylated Type 2 lactosamines; and (ii) contacting the cells with a glycosyltransferase and a donor nucleotide sugar, wherein the glycosyltransferase and donor nucleotide sugar are effective to enforce expression of the HCELL glycoform of CD44 on the cell.

In some embodiments, the passaging comprises contacting the cells with a xenogeneic or non-xenogeneic protease effective to lift adherent cells from a culture plate. In some embodiments, the protease is a recombinant protease selected from the group consisting of TrypLE Select Gibco Life Technologies), TrypLE (Gibco Life Technologies), rTrysin (Novozymes), recombinant Trypsin (MedxBio), TrypZean (Sigma-Aldrich), and combinations thereof.

In some embodiments, the cells are human mesenchymal stem cells (hMSCs).

In some embodiments, the cells are passaged 3 to 5 times. In some embodiments, the total number of cells is at least 1×10⁷ after the passaging step (i).

In some embodiments, the glycosyltransferase is an α(1,3)-fucosyltransferase. In some embodiments, the glycosyltransferase is α(1,3)-fucosyltransferase VI, α(1,3)-fucosyltransferase VII, or a combination thereof.

In some embodiments, the supplement is a xenogeneic supplement. In some embodiments, the supplement is a non-xenogeneic supplement. In some embodiments, supplement is human platelet lysate (HPL). In some embodiments, the culture medium comprises 1% to 20% HPL v/v. In some embodiments, the culture medium comprises 5% HPL v/v.

In some embodiments, the cells are grown to a maximum of 70% confluency at each passage.

In some embodiments, the HCELL is stable for at least 48 hours at 4° C. In some embodiments, the method further comprises the step of (iii) freezing and then thawing the cells, wherein the cells stably express the HCELL after thawing.

According to some embodiments, the present disclosure provides a population of cells produced by the process of (i) passaging the cells at least one time with a culture medium comprising a supplement wherein the cells comprise cell surface CD44 and the supplement is effective to maintain or increase the amount of a CD44 glycoform comprising sialylated Type 2 lactosamines; and (ii) contacting the cells with a glycosyltransferase and a donor nucleotide sugar, wherein the glycosyltransferase and donor nucleotide sugar are effective to enforce expression of the HCELL glycoform of CD44 on the cells.

In some embodiments, the passaging comprises contacting the cells with a xenogeneic or non-xenogeneic protease effective to lift adherent cells from a culture plate. In some embodiments, the protease is a recombinant protease selected from the group consisting of TrypLE Select Gibco Life Technologies), TrypLE (Gibco Life Technologies), rTrysin (Novozymes), recombinant Trypsin (MedxBio), TrypZean (Sigma-Aldrich), and combinations thereof.

In some embodiments, the cells are human mesenchymal stem cells (hMSCs). In some embodiments, the cells are passaged 3 to 5 times. In some embodiments, the total number of cells is at least 1×10⁷ after the passaging step (i). In some embodiments, the cells are grown to a maximum of 70% confluency at each passage.

In some embodiments, the glycosyltransferase is an α(1,3)-fucosyltransferase. In some embodiments, the glycosyltransferase is α(1,3)-fucosyltransferase VI, α(1,3)-fucosyltransferase VII, or a combination thereof.

In some embodiments, the supplement is human platelet lysate (HPL). In some embodiments, the culture medium comprises 1% to 20% HPL v/v. In some embodiments, the culture medium comprises 5% HPL v/v.

In some embodiments, the HCELL glycoform of CD44 is stable for at least 48 hours at 4° C. In some embodiments, the process further comprises (iii) freezing and then thawing the cells, wherein the cells stably express the HCELL glycoform of CD44 after thawing.

According to some embodiments, the present disclosure provides a process for producing GMP-grade exofucosylated cells comprising: (a) providing cells with a culture medium comprising a supplement, wherein the cells comprise cell surface CD44 and the supplement is effective to maintain or increase the amount of a CD44 glycoform comprising sialylated Type 2 lactosamines; (b) expanding the cells in the culture medium; and (c) contacting the cells with a glycosyltransferase and a donor nucleotide sugar that are effective to enforce expression of the HCELL glycoform of CD44 on the cells.

In some embodiments, the expanding of step (b) comprises passaging the cells 3-5 times. In some embodiments, the total number of cells is at least 1×10⁷ after the expanding of step (b). In some embodiments, the cells are grown to a maximum of 70% confluency at each passage of the expanding of step (b).

In some embodiments, the expanding of step (b) comprises contacting the cells with a xenogeneic or non-xenogeneic protease effective to lift adherent cells from a culture plate. In some embodiments, the protease is a recombinant protease selected from the group consisting of TrypLE Select Gibco Life Technologies), TrypLE (Gibco Life Technologies), rTrysin (Novozymes), recombinant Trypsin (MedxBio), TrypZean (Sigma-Aldrich), and combinations thereof.

In some embodiments, the cells are human mesenchymal stem cells (hMSCs).

In some embodiments, the glycosyltransferase is an α(1,3)-fucosyltransferase. In some embodiments, the glycosyltransferase is α(1,3)-fucosyltransferase VI, α(1,3)-fucosyltransferase VII, or a combination thereof.

In some embodiments, the supplement is a xenogeneic supplement. In some embodiments, the supplement is a non-xenogeneic supplement. In some embodiments, the supplement is human platelet lysate (HPL). In some embodiments, the culture medium comprises 1% to 20% HPL v/v. In some embodiments, the culture medium comprises 5% HPL v/v.

In some embodiments, the glycosyltransferse enforces expression of the HCELL glycoform of CD44 that is stable for at least 48 hours at 4° C. on the cells. In some embodiments, the method further comprises (d) freezing and then thawing the cells, wherein the cells stably express HCELL glycoform of CD44 on the cells after thawing.

According to some embodiments, the present disclosure provides an in vitro method for making cells that stably express a CD44 glycoform comprising sialylated Type 2 lactosamines comprising the steps of: (a) passaging the cells at least one time with a culture medium comprising a supplement effective to maintain or increase the amount of a CD44 glycoform comprising sialylated Type 2 lactosamines; (b) contacting the cells with a glycosyltransferase and a nucleotide sugar donor; and (c) storing the cells at 4° C. or less; wherein an HCELL glycoform of CD44 is stably expressed and cell viability is maintained for at least 48 hours.

In some embodiments, at least 80% of the cells express the HCELL glycoform of CD44 after 48 hours. In some embodiments, at least 80% of the cells are viable after 48 hours. In some embodiments, at least 80% of the cells are viable and express the HCELL glycoform of CD44 after 48 hours.

In some embodiments, the supplement is a xenogeneic or non-xenogeneic supplement. In some embodiments, the supplement is human platelet lysate (HPL). In some embodiments, the culture medium comprises 1% to 20% HPL v/v. In some embodiments, the culture medium comprises 5% HPL v/v.

In some embodiments, the passaging of step (a) comprises contacting the cells with a xenogeneic or non-xenogeneic protease effective to lift the cells from a culture plate.

In some embodiments, the cells are human mesenchymal stem cells (hMSCs).

In some embodiments, the total number of cells is at least 1×10⁷ after the passaging of step (a).

In some embodiments, the method further comprise (d) freezing and then thawing the cells, wherein the cells stably express the HCELL glycoform of CD44 after thawing.

According to some embodiments, the present disclosure provides a method of treating a subject in need thereof with GMP-grade exofucosylated human mesenchymal stem cells (hMSCs) comprising administering to the subject a therapeutically effective amount of GMP-grade exofucosylated hMSCs.

According to some embodiments, the present disclosure provides a system for producing Good Manufacturing Practices (GMP)-grade exofucosylated cells comprising: (a) cells comprising cell surface CD44; (b) a GMP-grade culture medium for passaging the cells comprising a supplement wherein the supplement is effective to maintain or increase the amount of a CD44 glycoform comprising sialylated Type 2 lactosamines; and (c) contacting the cells with a GDP-fucose donor and a glycotransferase selected from the group consisting of α(1,3)-fucosyltransferase III, IV, V, VI, VII, IX or a combination thereof.

In some embodiments, the glycosyltransferase is effective to enforce the HCELL glycoform of CD44 on the cells.

In some embodiments, the cells are human mesenchymal stem cells (hMSCs).

In some embodiments, the supplement is human platelet lysate (HPL). In some embodiments, the culture medium comprises 1% to 20% HPL v/v. In some embodiments, the culture medium comprises 5% HPL v/v.

In some embodiments, the supplement is effective to maintain expression of the CD44 glycoform comprising sialylated Type 2 lactosamines for at least 48 hours at 4° C. or less. In some embodiments, the supplement is effective to maintain viability of the cells for at least 48 hours at 4° C. or less. In some embodiments, the cells stably express the HCELL glycoform of CD44 on the hMSCs after freezing and then thawing the cells.

According to some embodiments, the present disclosure provides a method of selecting media supplements that are effective to maintain or increase the amount of a sialylated Type 2 lactosamine on a cell comprising the steps of: (a) contacting the cell with a culture medium comprising a supplement; (b) contacting the cell with a glycosyltransferase and a donor nucleotide sugar, wherein the glycosyltransferase and donor nucleotide sugar are effective to enforce expression of the glycan sLe^{X}; and (c) detecting the increased expression of the sLe^{X} glycan.

According to some embodiments, the present disclosure provides a method of identifying culture supplements that are effective to maintain or increase the amount of a sialylated Type 2 lactosamine on a cell comprising the steps of: (a) culturing a first population of cells using a first culture supplement; (b) culturing a second population of cells using a second culture supplement; (c) contacting the first and second populations of cells with a glycosyltransferase and a donor nucleotide sugar, wherein the glycosyltransferase and donor nucleotide sugar are effective to enforce expression of a glycan; and (d) detecting the glycan on the first and second population of cells.

In some embodiments, the first and second population of cells are passaged at least one time in the culture medium. In some embodiments, the first and second population of cells are stored at 4° C. or less for at least 24 hours after the contacting with glycosyltransferase of step (c) and prior to detecting the sLe^{X} on the populations of cells of step (d). In some embodiments, the first and second population of cells are frozen and then thawed after the contacting with glycosyltransferase of step (c) and prior to detecting the sLe^{X} on the population of cells of step (d).

In some embodiments, the method further comprises the step of (e) detecting the viability of the cells.

In some embodiments, the first and/or second supplement is selected from the group consisting of human platelet lysate (HPL) and fetal bovine serum (FBS).

In some embodiments, the glycosyltransferase is an α(1,3)-fucosyltransferase. In some embodiments, the glycosyltransferase is α(1,3)-fucosyltransferase VI, α(1,3)-fucosyltransferase VII, or a combination thereof.

In some embodiments, the cells are stored at 4° C. for at least 24 hours after the contacting of step (c). In some embodiments, the cells are frozen after the contacting of step (c).

In some embodiments, the cells are human mesenchymal stem cells (hMSCs).

In some embodiments, at least some of the sialylated Type 2 lactosamines are present as a CD44 glycoform comprising the sialylated Type 2 lactosamines.

In some embodiments, the glycosyltransferase of step (c) is effective to enforce the HCELL glycoform of CD44 on the first and second population of cells.

In some embodiments, the sLe^{X} glycan is detected by western blot. In some embodiments, the sLe^{X} glycan is detected by flow cytometry.

According to some embodiments, the present disclosure provides a method of selecting media supplements that are effective to maintain or increase the amount of terminal Type 2 lactosamines on a cell comprising the steps of: (a) contacting the cell with a culture medium comprising a supplement; (b) contacting the cell with a glycosyltransferase and a donor nucleotide sugar, wherein the glycosyltransferase and donor nucleotide sugar are effective to enforce expression of one or more of sLe^{X}, Le^{X}, VIM-2, and Difucosyl sLe^{X}; and (c) detecting one or more of sLe^{X}, Le^{X}, VIM-2, and Difucosyl sLe^{X} on the cell.

According to some embodiments, the present disclosure provides a method of selecting media supplements that are effective to maintain or increase the amount of a terminal Type 2 lactosamine on a cell comprising the steps of: (a) culturing a first population of cells using a first culture supplement; (b) culturing a second population of cells using a second culture supplement; (c) contacting the first and second populations of cells with a glycosyltransferase and a donor nucleotide sugar, wherein the glycosyltransferase and donor nucleotide sugar are effective to enforce expression of one or more of sLe^{X}, Le^{X}, VIM-2, and Difucosyl sLe^{X}; and (d) detecting one or more of sLe^{X}, Le^{X}, VIM-2, and Difucosyl sLe^{X} on the first and second population of cells.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1A to 1D shows FTVI- and FTVII-mediated α(1,3)-exofucosylation equally create sLe^{X} determinants on hMSCs. FIG. 1A shows representative flow cytometry histograms of HECA-452 mAb staining of RPMI 8402 cells that were buffer-treated (dotted line) or treated either with FTVI (black line) or FTVII (dashed line). HECA-452 staining of KG1a cells serves as a positive control. FIG. 1B shows representative flow cytometry histograms of HECA-452 (left), CSLEX-1(sLe^{X}; CD15s) (middle) and anti-CD15 (Le^{X}) (right) mAb staining on hMSCs cultured in media supplemented with FBS (top panels) or HPL (bottom panels) that were buffer-treated (dotted line) or exofucosylated with either FTVI (black line) or FTVII (dashed line). Gray filled histograms represent staining with isotype control. As assessed by HECA-452 and CSLEX-1 staining, hMSCs natively lack sLe^{X} epitopes (which are created after both FTVI and FTVII treatment) and Le^{X} fucosylated epitopes (which are created by FTVI treatment but not by FTVII). FIG. 1C shows flow cytometry analysis of HECA-452, CSLEX-1, anti-CD15 and anti-CD44 mAb staining of buffer-treated, FTVI-treated and FTVII-treated hMSCs cultured in media supplemented with FBS (gray bars) or HPL (black bars) (mean±SD; n=4). Statistically significant differences were assessed using paired t-test (**p<0.01). FIG. 1C shows representative western blot analysis of whole cell lysates of buffer-treated, FTVI-treated and FTVII-treated hMSCs resolved by SDS-PAGE and stained with HECA-452 mAb, E-selectin chimera (E-Ig), or anti-CD44 mAb. Lysates of KG1a cells serve as positive controls for HECA-452, E-Ig and CD44 blot results. Exofucosylation of hMSCs either with FTVI or FTVII engenders a HECA-452 and E-Ig-reactive glycoprotein of mw ˜90 kDa, which is CD44. For all figures, data are representative of four independent experiments.

FIG. 1E shows Flow Cytometry Mean Channel Fluorescence Intensity (MFI) levels of CD44 and of sLe^{X} (CD15s) and Le^{X} (CD15) determinants following FTVI- and FTVII-mediated α(1,3)-exofucosylation of hMSCs. Each panel shows mean±SEM of MFI levels of buffer-treated (BT), FTVI-treated and FTVII-treated hMSCs stained with HECA-452 mAb, and with anti-CD44, anti-CD15 and anti-CD15s (CSLEX-1) mAbs. Gray bars are cells cultured with FBS, dark bars are cells cultured with HPL. As shown, absolute MFI levels do not differ significantly in hMSCs cultured in HPL compared to FBS after α(1,3)-fucosylation using either FTVI or FTVII, but the baseline (buffer-treated) staining levels differ. As such, corrected for baseline variation, HPL shows higher FT-induced expression of CD15, CSLEX-1 (CD15s), and HECA452-reactive determinants (see FIG. 1C). As such, compared to FBS-supplementation, HPL-supplementation boosts expression of both sialylated and unsialylated Type 2 lactosaminyl glycans. Data are representative of experiments performed on hMSCs cultures derived from 10 different individuals. Statistically significant differences were assessed using paired t-test (*p<0.5; **p<0.01; ***p<0.001).

FIG. 2A to 2E shows validation of a manufacturing process for production of clinical-grade exofucosylated hMSCs. FIG. 2A shows flow cytometry histograms compared between untreated, buffer-treated, FTVI-treated and FTVII-treated hMSCs cultured under HPL conditions Immunophenotypic characterization was undertaken using a panel of cell surface markers including those associated with stromal, endothelial and hematopoietic cells. The values represent percentage of cells (mean±SD) that stained for respective markers. Data are representative of experiments performed on hMSCs cultures derived from at least 10 individuals. FIG. 2B shows flow cytometry analysis of anti-CD44 and HECA-452 mAb staining of buffer-treated, FTVI-treated and FTVII-treated hMSCs. As shown, α(1,3)-fucosylation with either FTVI and FTVII equally generates HECA-452 reactivity on hMSCs. Data are representative of experiments performed on hMSCs cultures derived from at least 10 individuals. Statistically significant differences were calculated using paired t-test (***p<0.001). FIG. 2C shows flow cytometry histograms compared between buffer-treated, FTVI-treated and FTVII-treated hMSCs based on their cell viability percentage after annexin V/PI staining. Graph values represent percentage of cells. FIG. 2D shows differentiation potential of buffer-treated (left panels) versus FTVI-treated hMSCs (right panels). Osteoblast formation was assessed by NBT/BCIP and Alizarin Red, and intracellular lipids droplets enrichment of hMSCs by Oil Red staining. Inset shows labelling of lipid vacuoles at higher magnification. Scale bars, 20 μm. FIG. 2E shows human PBMC (lymphocyte) binding to hMSCs as assessed by Stamper-Woodruff assay. hMSC binding of lymphocytes (bright circles) is markedly increased among FTVI-treated (bottom micrograph) compared to buffer-treated hMSCs (top micrograph). Insets show adhered lymphocytes at higher magnification. Scale bars, 20 μm.

FIG. 2F to 2G shows E-Ig staining and anti-CD44 mAb staining of CD44 immunoprecipitated from untreated and FTVI-treated hMSCs. CD44 immunoprecipitation was performed on whole cell lysates of untreated and FTVI-treated hMSCs cultured with HPL supplementation. FIG. 2F and FIG. 2G show representative western blot analysis of total cell lysate (T) and CD44 immunoprecipitated (IP) resolved by SDS-PAGE and stained with E-Ig (FIG. 2F) or anti-CD44 mAb (FIG. 2G). Western blot reveals a principal E-Ig-reactive glycoprotein of mw ˜90 kDa after α(1,3)-exofucosylation (FIG. 2F), which is created exclusively on the CD44 protein scaffold (˜90 kDa) on hMSCs (FIG. 2G).

FIG. 3A to 3E shows stability of exofucosylated hMSCs. FIG. 3A shows kinetics of cell viability (measured by flow cytometry as the percentage of annexin-V⁻PI⁻ cells) of buffer-treated vs FTVI-treated hMSCs stored either at RT or at 4° C. (mean±SD; n=3). FIGS. 3B and 3C shows kinetic analyses of HECA-452 expression by flow cytometry in the same samples as in FIG. 3A, showing the histograms of a representative sample (FIG. 3B). FIG. 3D to 3E shows kinetics of cell viability (FIG. 2D) and HECA-452 expression (FIG. 2E) of FTVII-treated hMSCs and stored at 4° C.; Data are representative of experiments performed on hMSCs cultures derived from 3 individuals.

FIG. 3F to 3H shows exofucosylation-enforced sLeX expression persists with cryopreservation, and for 24 hours after reculturing of thawed hMSCs. Representative flow cytometry histograms are shown for mAb HECA452 staining of FTVI-treated hMSCs. FIG. 3F shows data immediately following exofucosylation of hMSCs that were then cryopreserved; FIG. 3G shows data upon thawing of exofucosylated hMSCs that were cryopreserved and stored in liquid nitrogen for 24 h; and FIG. 3H shows data following 24 h of re-culture of the thawed exofucosylated hMSCs in HPL-containing media, detached using TrypLE Select reagent. Numbers shown under histograms 3F-3H are values of mean channel fluorescence.

FIG. 4A to 4C shows expression of c-Myc in exofucosylated hMSCs. Relative expression of c-Myc was measured by qRT-PCR and expressed as 2^{-ΔΔCt}. FIG. 4A shows C-Myc expression in a panel of 5 human cell lines and untreated hMSCs (n=13); mean±SD is depicted for the latter. DG75 was set as reference (2^{-ΔΔCt}=1). FIG. 4B shows C-Myc expression of buffer-treated or FTVI-treated hMSCs (mean±SD; n=4). FIG. 4C shows C-Myc expression of buffer-treated or FTVII-treated hMSCs (mean±SD; n=4).

### DETAILED DESCRIPTION

The following abbreviations are used herein: AP, alkaline phosphatase; BM, bone marrow; hMSCs, bone marrow-derived human mesenchymal stem cells: BMMCs, bone marrow mononuclear cells; CB, cord blood; CPD, cumulative population doubling; FBS, fetal bovine serum; FTVI, fucosyltransferase VI; FTVII, fucosyltransferase VII; GEP, gene expression profile; GMP, good manufacturing practice; GPS, glycosyltransferase-programmed stereosubstitution; HCELL, hematopoietic cell E-/L-selectin ligand; hMSCs, human mesenchymal stem cells; HPL, human platelet lysate; HSC, hematopoietic stem cell; ISCT, International Society for Cellular Therapy; PB, peripheral blood; PBMC, peripheral blood mononuclear cells; qPCR, quantitative PCR; RT, room temperature; RTK, receptor tyrosine kinase; Le^{X}, Lewis X; sLe^{X}, sialyl Lewis X.

Methods of Identifying and/or Selecting Cell Culture Conditions that are Effective to Enforce a Desired Amount of a Sialylated Type 2 Lactosamine

The present disclosure provides methods of identifying culture conditions that are effective to enforce a desired amount of sialylated Type 2 lactosamine on a cell. According to some embodiments, this is accomplished by using the accurate and high-sensitivity detection of terminal lactosaminyl glycans displayed on any glycolipid or glycoprotein, including those on cell surfaces, i.e., pertinent glycoprotein or glycolipid molecules/scaffolds on cell membranes. In some embodiments, the fidelity in target recognition of glycosyltransferases (GTs) (i.e. their stereospecificity for unique oligosaccharide compositions containing distinguishable monosaccharide sequences and anomeric linkage(s)) are exploited for their use as tools/reagents to identify the expression of key terminal lactosaminyl glycan determinants on glycolipid and glycoprotein scaffolds. Accordingly, the product of the glycosylation reaction(s) is measured by antibody and/or lectin probes specific for that product, and the generation of that product provides evidence that the acceptor structure (i.e., the glycan of interest) is expressed on the cell surface, or on any given glycolipid or glycoprotein substrate. Using this approach, the underlying acceptor structure can be detected without need for technically demanding glycoanalytic techniques such as mass spectrometry and nuclear magnetic resonance. Thus, the disclosed method provides an alternative to the current complex, technologically daunting practices in lactosaminyl glycan analysis, allowing for broadly accessible, high-throughput and cost-effective elucidation and tracking of lactosaminyl glycans present on soluble glycolipids or glycoproteins, or as displayed by cells, relevant to human health and disease.

In some embodiments, the above-described stereospecific exoglycosylation approach is used to identify effects of tissue culture conditions and/or reagents on the expression of terminal lactosaminyl glycans on a cell, or on glycoproteins or glycolipids that are produced by the cell. According to some embodiments, the present disclosure provides a method of detecting a change of expression of Type 2 terminal lactosamines on a population of cultured cells comprising the steps of: (a) contacting the cells with a glycosyltransferase (e.g. a fucosyltransferase) and a donor nucleotide sugar, wherein the glycosyltransferase and donor nucleotide sugar are effective to enforce expression of a glycan (e.g., sLe^{X}, Le^{X}, VIM-2, and Difucosyl sLe^{X}); and (b) detecting the product glycan on the cells, wherein the contacting of step (a) is performed before and/or after any culture condition modification. The cells that are contacted with the glycosyltransferase may be any of the cell types disclosed herein, and the expression of Type 2 terminal lactosamines and the resulting product glycans may be present on the cell surface, within the cell, or secreted by the cell. The detection of product glycan may be performed on whole (i.e. intact) cells, on the components of disrupted cells, or on components released by cells (e.g. glycoconjugates secreted from the cell, released as particles from the cells, or cleaved off the surface of a cell).

In some embodiments, the capacity to enforce the expression of a lactosaminyl glycan on a given glycoconjugate using a glycosyltransferase is independent of the scaffold (e.g. lipid or protein) on which the glycan is enforced. This is because the target of the glycosyltransferase may be the glycan on the glycoconjugate, without preference for the scaffold upon which the glycan is displayed. In some embodiments, the glycans are enforced on a limited number of glycoconjugates, consisting of one or more of glyolipids, glycoproteins, and combinations thereof. In some embodiments, expression of a lactosaminyl glycan is enforced on one or more of the following scaffolds: glycolipids (e,g, neolactose-series glycosphingolipids), CD44 glycoprotein, CD43 glycoprotein, PSGL-1 (CD162) glycoprotein, CD34 glycoprotein, ESL-1/Glg1 glycoprotein, Myeloperoxidase glycoprotein, CD34 glycoprotein, L-selectin (CD62L) glycoprotein, CD66 (CEA; CEACAM) glycoproteins, CD11a and/or CD18 glycoproteins (LFA-1). In some embodiments, a fucosyltransferase may be used to enforce expression of the lactosaminyl glycan. For example, an α(1,3)-fucosyltransferase 3 (FT3), FT5, FT6, FT7, and combinations thereof may be used to enforce expression of sLe^{X} on the scaffold lipid and/or protein (i.e., glycolipid and/or glycoprotein). In another example, FT3, FT4, FT5, FT6, FT9, and combinations thereof may be used to enforce Le^{X} expression.

Although several of the various embodiments comprise enforcement of Type 2 lactosaminyl glycans (e.g. sLe^{X} and Le^{X}), it should be understood that any lactosaminyl glycan acceptor that can be distinctively targeted by a glycosyltransferase may be probed using the stereospecific exoglycosylation approach provided herein. For example, stereospecific modifications of Type 1 lactosaminyl glycans may be enforced (e.g. Le^{e}, H type I, Lea, Type I SialylLacNAc, SLe^{A}) using fucosyltransferases (e.g. FT1, 2, 3, and 5) and/or sialyltransferase (e.g., ST6Gal1 (which can be used to detect Type 2 lactosamines displayed on N-glycans) or ST3Gal 3, 4, and 6 which can each modify Type 1 or Type 2 lactosamines) using the same stereospecific exoglycosylation approach provided herein, with detection of the product glycan of the exoglycosylation reaction. Indeed, with regard to modifications of subterminal GlcNAc, the fucosyltransferases FT3 and FT5 can fucosylate GlcNAc at both the 3 and 4 position (i.e., these enzymes are “α(1,3/4)-FTs”), thus can be used to detect acceptors that are Type 2 lactosamines or Type 1 lactosamines, respectively.

In some embodiments, the detection of the product glycan comprises an antibody-based technique, wherein the antibody has reactivity to the product glycan. Any antibody may be used to probe for the product glycan, including, without limitation, the antibodies described herein. In some embodiments, the antibody based technique is selected from the group consisting of enzyme-linked immunosorbent assay (ELISA), western blot, immunohistochemistry, immunocytochemistry, immunofluorescence, flow cytometry, immunoprecipitation, and combinations thereof. Many other antibody-based techniques are known by those skilled in the art, which may be used to detect the product glycan.

In some embodiments, the detection of the product glycan comprises a lectin-based technique, wherein a lectin is used to probe for the presence of a product glycan. Any lectin may be used, including the Ca++ dependent lectins such as selectins (e.g. E-selectin), or DC-SIGN (which recognizes Le^{X}), or other lectins that recognize sialylated lactosamines after treatment of the terminal lactosaminyl-bearing glycoconjugate with a sialyltransferase and CMP-sialic acid donor (e.g., siglecs, SNA, MALII, etc).

In some embodiments, the detection of the product glycan comprises a tag-based technique comprising a tag-modified sugar incorporated into the product glycan. In some embodiments, glycans can be detected by incorporation of a modified sugar donor onto the underlying glycan structure. In some embodiments, directed placement upon a cell surface of a sugar donor (e.g. a GDP-fucose donor) is performed, wherein the sugar donor is modified with a chemically-reactive tag (e.g., a functional group serving as a chemical reporter) which would then allow subsequent conjugation with another structure (e.g., via a bioorthogonal chemical reaction) and/or wherein the sugar donor is linked (modified covalently) prior to introduction onto the cells with one or more additional molecules that confer a desired property (e.g. a biologic property). In such embodiments, the use of a glycosyltranfersae (e.g. α(1,3)-FTs) confers both regiospecificity and stereospecificity in the placement of the pertinent molecular moiety which is linked to the installed sugar donor (e.g. fucose). In some embodiments, the modified (functionalized) sugar donor comprises a uniquely reactive chemical handle that is displayed on the cell surface after the modified sugar is introduced onto a cell (e.g. via fucosyltransferase). The chemical handle will react only when exposed to a reagent or moiety that has a matched reactivity. Thus, according to some embodiments, the modified sugar donor may be engineered to bear any molecule having (or engineered to have) the concomitant reactivity. According to some embodiments, the modified sugar donor is effective for chemoselective-ligation reactions that are well known in the art to selectively form a covalent linkage in a biological medium. According to some embodiments, stereospecific addition of a molecular tag-modified donor nucleotide allows for subsequent linkage of other molecules onto the installed sugar in a distinct pattern onto lactosaminyl glycans. In some embodiments, molecules containing distinct properties can be covalently linked to the donor nucleotide and can thus be stereospecifically added to lactosaminyl glycans. For example, molecules containing distinct biological properties can be covalently linked to fucose incorporated within sLe^{X}. In some embodiments, the functionalized sugar (e.g. fucose) used in conjunction with one or more glycosyltransfeases (e.g. fucosyltransferase) is an azide- or alkyne-tagged sugar. For example, in some embodiments a GDP-azido-fucose is used. Any azido-fucose analogue known in the art may be used (e.g., GDP-Azido-Fucose, R&D Systems, Bio-Techne Corporation, Cat. No. ES101-100). According to some embodiments, fucose-alkyne is used. Any fucose-alkyne analogue known in the art may be used (e.g. Click-IT Fucose Alkyne, Thermo Fisher, At. No. C10264) According to some embodiments, the alkyne or azide tagged fucose is further conjugated to another molecule. For example, after depositing an azido-fucose onto the surface of a cell using one or more fucosyltransferases, the azido fucose may be conjugated to biotinylated alkyne. The resulting covalently bound biotinylated fucose may then be used to attach any avidin/streptavidin-bound molecule known in the art. Addition of a donor GDP-fucose wherein the fucose has been modified by methods known in the art with a chemical reactive group/molecular tag (e.g., biotinylated GDP-fucose, azido-GDP-fucose, etc.) thereby allowing for subsequent linkage of other molecules onto the installed fucose within cell surface lactosaminyl glycans (examples of this approach include, but are not limited to, utility of biotinylated GDP-fucose with subsequent complexing using streptavidin-conjugated molecules and/or use of “click chemistry” wherein the azido-containing fucose molecule is then complexed to an alkyne-containing molecule). In other embodiments, molecules covalently linked to the donor nucleotide fucose (i.e., GDP-fucose with covalent attachment of additional molecule(s)) can be stereospecifically added in a distinct pattern onto cell surface lactosaminyl glycans to endow a desired biologic property upon the cell. According to some embodiments, the conjugation of azido-fucose to a biotin moiety is performed via copper mediated click chemistry, as known in the art. For example, for each reaction, 20 nmol of Cu2+, 10 nmol of biotinylated alkyne and 200 nmol of ascorbic acid may be combined at room temperature to allow the Cu2+ to reduce to Cu+. The mixture may then be diluted in 25 mM Tris, 150 nM NaCl at pH 7.5, and then applied for 30 minutes to cells having azido-fucose deposited on the cell surface (e.g., by exofucosylation). The reaction solution may then be removed and cells washed. The resulting biotinylated fucose may then be further conjugated to additional molecules of interest via interaction with biotin. The structures that may be conjugated to the modified sugar donor after being deposited on the cell surface according to embodiments disclosed herein include, but are not limited to, peptides, proteins, nucleotides, polynucleotides, carbohydrates, lipids, antibodies (such as IgA, IgD, IgE, IgG, IgM, and fragments thereof), probes, and combinations thereof.

According to some embodiments, the present disclosure provides a method of detecting differences in level of expression of cell-surface Type 2 terminal lactosamines on a population of cultured cells propagated under different conditions comprising the steps of: (a) culturing a first population of cells under a first culture condition; (b) culturing a second population of cells under a second (different) culture condition; (c) contacting the first and second population of cells with a glycosyltransferase and a donor nucleotide sugar, wherein the glycosyltransferase and donor nucleotide sugar are effective to enforce expression of a glycan; and (d) detecting the glycan on the first and second population of cells.

The conditions of cell culture that may be varied to detect differences in one or more embodiments include any parameter of cell culture known by those skilled in the art. The culture conditions include, without limitation, (1) any component of cell culture media (e.g., minerals, chemicals, molecules, cytokines, or growth supplements including those disclosed herein (e.g. supplements like HPL and FBS)), (2) the temperature at which cells are cultured, (3) the amount of oxygen (e.g. hypoxia or hyperoxia) or carbon dioxide under which the cells are cultured, (4) the presence of additional cell types (e.g. feeder cell layers), (5) structures in or upon which cells are grown (e.g. matrigel, extracellular matrix elements, or cells growth on scaffolds or on coated culture vessel surfaces), (6) the handling of the cells (e.g. the number of times passaged and the confluency of the cells) and combinations thereof.

The first population of cells and the second population of cells may, e.g., be the same cell type, different cell types, or start as the same cell type but change during culture, according to the various embodiments disclosed herein. For example, in some embodiments, cells are cultured under conditions which cause the cells to change during culturing, such as stem cells (e.g. induced pluripotent stem cells) being cultured under conditions that are effective to cause differentiation. As used herein the terms “first population” and “second population” are not necessarily limited in any chronological way (e.g. the first and second populations of cells may be cultured concurrently or consecutively).

According to some embodiments, the product glycans resulting from glycosytransferase treatment that are detected on the first and second population of cells may be compared to one another to identify the culture conditions that are effective to produce a desired amount of a Type 2 lactosaminyl glycan. In some embodiments, the differing culture conditions will result in a detectable difference (e.g. t-test p<0.05) in the amount of product glycan present on the cells after contacting with the glycosyltransferase. In some embodiments, culture conditions significantly increase the amount of Type 2 lactosaminyl glycan present on cells. In some embodiments, culture conditions significantly decrease the amount of Type 2 lactosaminyl glycan present on cells. In some embodiments, the significant increase or decrease in the amount of Type 2 lactosaminyl glycan is detected via product glycan after contacting with a glycosyltransferase. For example, in some embodiments, one or more detected amounts of the HCELL glycoform of CD44 on cells are compared to determine relative amounts of terminal sialylated Type 2 lactosamines on CD44 that have resulted from varying cell culture conditions.

According to some embodiments, populations of cells are tested for the ability to retain enforced expression of a product glycan and for cell viability. In some embodiments, the first and second population of cells are stored at 4° C. or less for at least 24 hours after the contacting with the glycosyltransferase. In some embodiments, the first and second population of cells are frozen and then thawed after the contacting with the glycosyltransferase. In some embodiments, the viability of cells is detected after the storage at 4° C. or less, or freezing and thawing.

According to some embodiments, the present disclosure also provides a method of identifying and/or selecting media supplements that are effective to maintain or increase the amount of a sialylated Type 2 lactosamines on a cell, such as a human mesenchymal stem cell (hMSC). In some embodiments, the method comprises the steps of: (a) contacting the cell with a culture medium comprising a supplement; (b) contacting the cell with a glycosyltransferase and a donor nucleotide sugar, wherein the glycosyltransferase and donor nucleotide sugar are effective to enforce expression of the glycan sLe^{X}; and (c) detecting the increased expression of the sLe^{X} glycan.

In some embodiments, the present disclosure provides a method of identifying and/or selecting media supplements that are effective to maintain or increase the amount of a sialylated Type 2 lactosamine on human mesenchymal stem cells (hMSCs) comprising the steps of: (a) culturing a first population of cells using a first culture supplement; (b) culturing a second population of cells using a second culture supplement; (c) contacting the first and second populations of cells with a glycosyltransferase and a donor nucleotide sugar, wherein the glycosyltransferase and donor nucleotide sugar are effective to enforce expression of a glycan; and (d) detecting the glycan on the first and second population of cells.

According to some embodiments, generated fucosylated lactosaminyl glycans, such as Le^{x}, sLe^{x}, VIM-2, and Difucosyl sLe^{X} can be detected by reactivity to one or more antibodies as described herein.

### Methods of Enforcing and Maintaining Cell Surface Glycosylation

The present disclosure is also directed to methods and compositions for enforcing and maintaining cell surface glycosylation on any cultured cell of interest. According to some embodiments, an in vitro method is provided for making human mesenchymal stem cells (hMSCs) comprising the steps of (i) passaging the hMSCs at least one time with a culture medium comprising a supplement, such as a non-xenogeneic supplement, and (ii) contacting the hMSCs with a glycosyltransferase, wherein the hMSCs comprise cell surface CD44 and the non-xenogeneic supplement is effective to increase the amount of a CD44 glycoform comprising sialylated Type 2 lactosamines. As used herein, the term “CD44 glycoform” means any of the several forms of CD44 glycoprotein that can exist on or in a cell, including those that are enforced, e.g. by exofucosylation. CD44 glycoforms include, but are not limited to, CD44 comprising sialylated Type 2 lactosamines and the HCELL glycoform of CD44, comprising CD44 whose sialylated Type 2 lactosamine acceptors have been fucosylated to create sLe^{X} determinants.

The term “mesenchymal stem cell” (MSC) refers to cells isolated from stroma, the connective tissue that surrounds other tissues and organs. MSCs express a panel of markers including, but not limited to, CD13, CD44, CD73, CD105. MSCs are postnatal stem cells capable of self-renewing and can differentiate into a variety of cells such as osteoblasts, chondrocytes, adipocytes, and neural cells. These cells typically express STRO-1, CD29, CD73, CD90, CD105, CD146, and SSEA4, but do not typically express hematopoietic cell markers, especially CD14 and CD34; however, MSCs derived from tissues other than marrow (e.g., from adipose tissue) and a subset of MSCs known as “pericytes” or “adventitial” cells can natively express CD34, and this marker is characteristically lost on culture-expansion. In some embodiments, the MSCs are cultured at low densities (i.e. less than 70% maximum confluency). The MSC could be unmodified or may be modified (e.g., by nucleic acid transfection to express a desired protein product of interest, by viral transduction, etc.).

The term “non-xenogeneic” means that the pertinent component is not obtained from an animal source other than that from which the host cell is derived (if the component is derived from the same animal, the component is “homologous”), or does not derive from any animal source. Xenogeneic components can elicit immune-reactivity (e.g. via incorporation of xeno-epitopes on human cells) or introduce infectious agents (e.g. pathogenic viruses and prions). For example, in some embodiments, the cells being cultured and passaged are human cells and the non-xenogeneic supplement is human platelet lysate (HPL).

The term “supplement” refers to any natural or artificial component added to the culture medium. In some embodiments, the supplement is non-xenogeneic. In some embodiments, the supplement is not non-animal-derived (e.g., could be created chemically or recombinantly). In some embodiments, the supplement is one or more of amino acids, minerals, organic molecules/compounds, small molecules, vitamins, salts, lipids, cytokines and protein polypeptides, and trace elements. In some embodiments, the supplement is one or more of human AB serum, thrombin-activated platelet release plasma, human platelet lysate, and pooled human platelet lysate. In some embodiments, the supplement is present in the culture medium in the amount of at least 1, 2, 3, 4, 5, 6, 7, 8, 9, 10, 15, 20%, or more v/v in culture medium. According to some embodiments, the amino acids include one or more of alanine, arginine, asparagine, aspartic acid, cysteine, glutamic acid, glycine, histidine, isoleucine, leucine, lysine, methionine, phenylalanine, proline, serine, threonine, tryptophan, tyrosine, valine and glutamine. In some embodiments, the cytokines and protein/polypeptides could include one or more of fibroblast growth factor 1 (FGF1), Interleukins (e.g., IL-1, IL-2, IL-4, IL-7, IL-10, IL-12, IL-15, IL-18, IL-21, etc.), fibroblast growth factor 2 (FGF2), epidermal growth factor (EGF), platelet derived growth factor (PDGF), insulin (insulin), insulin-like growth factor 1 (IGF1), vascular endothelial growth factor (VEGF), placental growth factor (PGF), Colony-Stimulating Factors (e.g., G-CSF, GM-CSF, M-CSF, Erythropoietin, Thrombopoietin), Leukocyte inhibitor factor (LIF), stem cell factor (SCF), transferrin (transferrin) and human serum albumin (HSA). In some embodiments, agents that stimulate production/activity of HIF-1a (e.g., desferroxamine). In some embodiments, the vitamins include one or more of biotin, choline chloride, D-pantothenic acid sodium, folic acid, inositol, niacinamide, riboflavin, pyridoxine hydrochloride, thiamine hydrochloride, coenzyme Q10, vitamin B12, Putrescine dihydrochloride, vitamin C and vitamin E. In some embodiments, the lipids include one or more of dexamethasone, oleic acid, cholesterol, ethanolamine, linoleic acid, lipoic acid and lipid mixture (Sigma, L5416). In some embodiments, the trace elements include one or more of cobalt chloride, sodium selenite, nickel chloride, manganese chloride, hexaammonium molybdate, aluminum chloride, chromium potassium sulfate, copper sulfate, ferric nitrate, ferrous sulfate and zinc sulfate. The supplement may comprise one or more other molecules, including antioxidants (β-mercaptoethanol, reduced glutathione), butyrate, D-glucose, taurine, heparin sodium, hyaluronic acid, etc.

According to some embodiments, the supplement comprises one or more of the following components

| Component | Concentration or Amount | |
|---|---|---|
| Ala (Alanine) | 0.01~0.25 | mM |
| Arg (Arginine) | 0.4~10 | mM |
| Asn (Asparagine) | 0.11~2.75 | mM |
| Asp (Asparagic acid) | 0.08~2 | mM |
| Cys (Cysteine) | 0.03~0.75 | mM |
| Glu (Glutamic acid) | 0.03~0.75 | mM |
| Gly (Glycine) | 0.05~1.25 | mM |
| His (Histidine) | 0.03~0.75 | mM |
| Ile (Isoleucine) | 0.5~12.5 | mM |
| Leu (Leucine) | 0.24~6 | mM |
| Lys (Lysine) | 0.2~5 | mM |
| Met (Methionine) | 0.12~3 | mM |
| Phe (Phenylalanine) | 0.21~5.25 | mM |
| Pro (Proline) | 0.004~0.1 | mM |
| Ser (Serine) | 0.15~3.5 | mM |
| Thr (Threonine) | 0.06~1.5 | mM |
| Trp (Tryptophan) | 0.02~0.5 | mM |
| Tyr (Tyrosine) | 0.03~0.75 | mM |
| Val (Valine) | 0.28~7 | mM |
| Gln (Glutamine) | 0.8~20 | mM |
| Epidermal growth factor (EGF) | 1-100 | μg/L |
| Fibroblast growth factor-1 (FGF1) | 1-100 | μg/L |
| Fibroblast growth factor-2 (FGF2) | 1-100 | μg/L |
| Insulin-like growth factor 1 (IGF1) | 1-100 | μg/L |
| Leukocyte inhibitor factor (LIF) | 1-100 | μg/L |
| Platelet derived growth factor (PDGF) | 1-100 | μg/L |
| Placental growth factor (PGF) | 1-100 | μg/L |
| Stem cell factor (SCF) | 1-100 | μg/L |
| Vascular endothelial growth factor | 1-100 | μg/L |
| (VEGF) | | |
| insulin | 1~25 | mg/L |
| transferrin | 4~100 | mg/L |
| Human serum albumin (HSA) | 0.02~0.5 | g/L |
| biotin | 6~150 | μM |
| Choline chloride | 12~300 | μM |
| Folic acid | 1.2~30 | μM |
| Coenzyme Q10 | 0.2~5 | μM |
| Dexamethasone | 2~50 | nM |
| D-pantothenic acid sodium | 1~25 | μM |
| i-Inositol | 14~350 | μM |
| Niacinamide | 4~100 | μM |
| Pyridoxine hydrochloride | 0.03~0.75 | μM |
| Riboflavin | 0.11~2.75 | μM |
| Thiamine hydrochloride | 0.11~2.75 | μM |
| Vitamin B12 | 0.1~2.5 | μM |
| Putrescine Dihydrochloride | 0.1~2.5 | μM |
| (Putresicine - 2HCl) | | |
| Vitamin C | 2~50 | mg/L |
| Vitamin E | 0.05~1.25 | mg/L |
| Heparin sodium | 0.08~2 | g/L |
| D-glucose | 3.6~90 | mM |
| Reduced glutathione | 0.0002~0.005 | mg/L |
| Taurine | 0.00144~0.36 | g/L |
| β-mercaptoethanol (BME) | 0.01~0.25 | μg/L |
| Phenol red | 1.62~40.5 | mg/L |
| Oleic acid | 0.5~12.5 | mg/L |
| Linoleic acid | 0.5~7.5 | mg/L |
| Lipoic acid | 0.02~0.5 | mg/L |
| Lipid mixture (Sigma, L5416) | 0.2~5 | ml/L |
| Cholesterol | 1~25 | mg/L |
| Ethanolamine | 12~300 | .mu.l/L |
| Sodium Bicarbonate (NaHCO₃) | 2.32~58 | mM |
| Calcium Chloride (CaCl₂) | 0.0168~0.42 | mM |
| Potassium Chloride (KCl) | 0.832~20.8 | mM |
| Magnesium Chloride (MgCl₂) | 0.06~1.5 | mM |
| Magnesium Sulfate (MgSO₄) | 0.0814~2.035 | mM |
| Sodium Chloride (NaCl) | 24.122~603.55 | mM |
| Sodium Dihydrogen | 0.0906~2.265 | mM |
| Phosphate Monohydrate (NaH₂PO₄—H₂O) | | |
| Sodium Phosphate Dibasic (Na₂HPO₄) | 0.1~2.5 | mM |
| Sodium pyruvate | 0.1~2.5 | mM |
| TNF ┘ | 0.1-100 | ng/mL |
| G-CSF | 0.1-100 | ng/mL |
| IL-15 | 0.1-100 | ng/mL |
| IL-21 | 0.1-100 | ng/mL |
| IL-1 | 0.1-100 | ng/mL |
| IL-2 | 0.1-100 | ng/mL |

According to some embodiments, the supplement is effective for the culture of any cell derived from human or mammalian tissues, including somatic cells, leukocytes (including leukocytic cells derived from culture of blood leukocytes, e.g., monocyte-derived dendritic cells), genetically altered and/or manipulated cells (e.g., chimeric antigen receptor (CAR)-T cells), embryonic stem cells, induced pluripotential stem cells (iPS cells), and tissue progenitor cells or stem cells, including all types of adult stem cells. In some embodiments, the supplement is effective for culture of adipose-derived mesenchymal stem cells, bone marrow-derived mesenchymal stem cells, and/or umbilical cord-derived stem cells. According to some embodiments, the supplement is effective to increase the expression of sialylated lactosamine-bearing CD44 on the surface of a cell. According to some embodiments, cells contacted with a supplement maintain, after contact with a fucosyltranferase, HCELL expression at 4° C. for at least 24 hours. According to some embodiments, cells contacted with a supplement maintain HCELL expression after being frozen. As used herein, “frozen” or “cryopreserved” (and grammatical variations thereof) cells means that the cells are frozen under conditions effective to maintain viability until the cells are thawed.

The term “passaging” or grammatical variations thereof refers to the process of growing a culture of cells in a culture medium by transferring all or some cells from a previous culture into the new culture with none, some or all fresh culture medium. In some embodiments, cells are grown to a maximum of 40% confluency at each passage. In some embodiments, cells are grown to a maximum of 50% confluency at each passage. In some embodiments, cells are grown to a maximum of 60% confluency at each passage. In some embodiments, cells are grown to a maximum of 70% confluency at each passage. In some embodiments, cells are grown to a maximum of 80% confluency at each passage. In some embodiments, cells are grown to a maximum of 90% confluency at each passage. According to some embodiments, the cells are passaged 1, 2, 3, 4, 5, 6 times or more. The term “expanding,” or grammatical variations thereof, refers to the process of growing a culture of cells in a culture medium. According to some embodiments, the passaging or expanding results in a total number of cells of at least 1×10⁶, 1×10⁷, 1×10⁸, 1×10⁹, or more.

The term “culture medium” or “growth medium” or grammatical variations thereof refers to a solid, liquid, or semi-solid effective to support the growth of cells. The culture medium may comprise any type of natural media component and/or artificial media component. Natural media components include, but are not limited to, biological fluids (e.g. plasma, serum, lymph, human placental cord serum, amniotic fluid), tissue extracts (e.g. extracts of liver, spleen, tumors, leucocytes, and bone marrow), and balanced salt solutions (e.g. PBS, DPBS, HBSS, EBSS). Artificial media components include, but are not limited to, basal media (e.g. MEM and DMEM) and complex media (e.g. RPI-1640, IMDM). In some embodiments, the culture medium comprises a mixture of amino acids, glucose, salts, vitamins, and other nutrients, and a buffering system for regulating pH (e.g. gaseous CO₂, HEPES). In some embodiments, the culture medium comprises serum to provide a source of amino acids, proteins, vitamins (such as fat-soluble vitamins A, D, E, and K), carbohydrates, lipids, hormones, growth factors, minerals, and trace elements. For example, serum may include fetal bovine serum, or calf bovine serum. In some embodiments, the culture medium comprises one or more antibiotics (e.g. penicillin/streptomycin).

In some embodiments, the passaging comprises contacting cells (such as human MSCs) with a composition, such as an enzyme (e.g. a non-xenogeneic protease), that is effective to lift the cells from a culture plate. In some embodiments, the composition includes, but is not limited to one or more of animal origin-free recombinant enzymes that are effective to dissociate cells from a growth surface. In some embodiments, the enzyme cleaves peptide bonds one the C-terminal side of lysine and arginine. For example, in some embodiments, the passaging of cells comprises contacting cells with a composition comprising one or more of TrypLE (Gibco Life Technologies), rTrysin (Novozymes), recombinant Trypsin (MedxBio), TrypZean (Sigma-Aldrich).

According to some embodiments, cells of the disclosed invention are contacted with a glycosyltransferase to enforce a glycan on the cell surface. According to some embodiments, fucosylated lactosaminyl glycans are enforced by a member of the α(1,3)-fucosyltransferase family. The α(1,3)-fucosyltransferase family includes Fucosyltransferase III (also called FTIII, FT3, FUTIII, FUT3), Fucosyltransferase IV (also called FTIV, FT4, FUTIV, FUT4), Fucosyltransferase V (also called FTV, FT5, FUTV, FUT5), Fucosyltransferase VI (also called FTVI, FT6, FUTVI, FUT6), Fucosyltransferase VII (also called FTVII, FT7, FUTVII, FUT7), Fucosyltransferase IX (also called FTIX, FT9, FUTIX, FUT94), and variants thereof. The cDNA/protein sequences for the α(1,3)-fucosyltransferase family are as follows:

TABLE I

| Name | GenBank Acc. No. | ID NO: |
|---|---|---|
| Fucosyltransferase III | BC108675 | SEQ ID NO: 1 (AA) |
| (FUT3; FT3) | | SEQ ID NO: 2 (cDNA) |
| Fucosyltransferase IV | BC136374 | Long |
| (FUT4; FT4) | | SEQ ID NO: 3 (AA) |
| | | SEQ ID NO: 4 (cDNA) |
| | | Short |
| | | SEQ ID NO: 5 (AA) |
| | | SEQ ID NO: 6 (cDNA) |
| Fucosyltransferase V | BC140905 | SEQ ID NO: 7 (AA) |
| (FUT5; FT5) | | SEQ ID NO: 8 (cDNA) |
| Fucosyltransferase VI | BC061700 | SEQ ID NO: 9 (AA) |
| (FUT6; FT6) | | SEQ ID NO: 10 (cDNA) |
| Fucosyltransferase VII | BC074746 | SEQ ID NO: 11 (AA) |
| (FUT7; FT7) | | SEQ ID NO: 12 (cDNA) |
| Fucosyltransferase IX | BC036101 | SEQ ID NO: 13 (AA) |
| (FUT9; FT9) | | SEQ ID NO: 14 (cDNA) |

As used herein, the notation for a fucosyltransferase should not be construed as limiting to the nucleotide sequence or the amino acid sequence. For example, the notation of Fucosyltransferase IX, FTIX, FT9, FUTIX or FUT9 are used interchangeably as meaning the nucleotide, amino acid sequence, or both, of Fucosyltransferase IX. According to some embodiments, cells are contacted by one or more of the α(1,3)-fucosyltransferase family members to enforce fucosylated lactosaminyl glycans.

As used herein, the term “contact” (and grammatical variations thereof) means to bring two things within physical proximity or to physically touch. In one non-limiting example, the term “contact” (and grammatical variations thereof) of an enzyme with a cell to enforce glycans includes any form of bringing an enzyme into proximity with its substrate so as to allow for enzymatic activity. For example, cells contacted by one or more α(1,3)-fucosyltransferase family members to enforce a fucosylated lactosaminyl glycans includes, but is not limited to, direct contact with an α(1,3)-fucosyltransferase together with donor nucleotide sugar GDP-fucose with cell surface substrates (i.e., exofucosylation), and also includes contact of the α(1,3)-fucosyltransferase with intracellular substrates by any means of introducing nucleic acid (e.g., transfection, electroporation, transduction) encoding the α(1,3)-fucosyltransferase into a cell. The contacting can be together (i.e., introducing a nucleic acid encoding a given fucosyltransferase together with cell surface exofucosylation using the same (or another) fucosyltransferase). In another non-limiting example, a cell may “contact” a supplement by the addition of the supplement to the culture media in which the cell is grown.

In some embodiments, fragments of α(1,3)-fucosyltransferase family members are contacted with a cell. For example, a peptide/nucleotide having at least 50%, 60%, 70%, 80%, 90%, 95%, 96%, 97%, 98%, or 99% identity to an α(1,3)-fucosyltransferase family member is contacted with a cell. As used herein, the term “identity” and grammatical versions thereof means the extent to which two nucleotide or amino acid sequences have the same residues at the same positions in an alignment. Percent (%) identity is calculated by multiplying the number of matches in a sequence alignment by 100 and dividing by the length of the aligned region, including internal gaps.

According to some embodiments, the fucosylated lactosaminyl glycans can be detected by reactivity to one or more antibodies. For example, the sLe^{x}, Le^{x}, VIM-2, and Di-Fuc-sLe^{X} may be detected by one or more of the HECA-452 antibody (ATCC HB-11485) and/or CSLEX1 antibody (clone CSLEX1, BD Pharmingen, Billerica, Mass.), an anti-Le^{x} antibody such as the H198 mAb (clone H198, Biolegend, San Diego, Calif.), the VIM-2 antibody (clone VIM-2, BioRad), and an anti-Di-Fuc-sLe^{X} antibody such as the FH6 mAb (clone FH6, BioLegend), respectively. Other antibodies known in the art that bind to sLe^{X}, Le^{x}, VIM-2, and Di-Fuc-sLe^{X} motifs may also be used. E-selectin (e.g., E-selectin-immunoglobulin chimera) could also be used to detect either sLe^{X}, VIM-2, or Di-Fuc-sLe^{x}

In some embodiments, the disclosed compositions and methods are effective to retain/maintain the level of a CD44 glycoform during culture or to increase the amount of a CD44 glycoform comprising sialylated Type 2 lactosamines, which may be detected after contacting with a glycosyltransferase (to produce the products of the glycosylation, e.g., Le^{x}, sLe^{X}, VIM-2, and Difucosyl sLe^{X}). The term “increase”, and grammatical variations thereof, as used to describe an increase in the amount of a glycan or CD44 glycoform (e.g. HCELL), means that there is a statistically significant increase in the amount of a glycan or CD44 glycoform relative to a control. In some embodiments, one or more of ANOVA, t-tests, F-tests, among others, may be used to determine the statistical significance of a glycan measurement. According to some embodiments, if the cells are measured to have statistically significantly more glycan or CD44 glycoform (e.g. HCELL) compared to values obtained from that of a negative control by t-test (p<0.05), the glycan or CD44 glycoform is increased in the cells. For example, in some embodiments, the amount of a glycan or a CD44 glycoform (e.g. HCELL) is measured as mean fluorescence intensity (MFI) units by flow cytometry. In some embodiments, the cells cultured with a supplement have a statistically significant increase in the amount of a CD44 glycoform (e.g. HCELL) after contact with a fucosyltransferase relative to cells with no supplement or with a different supplement, t-test (p<0.05), as measured by MFI with flow cytometry. In some embodiments, cells cultured with HPL have a statistically significant increase in the amount of a glycan or CD44 glycoform (e.g. HCELL) relative to cells cultured with FBS, t-test (p<0.05), as measured by MFI with flow cytometry.

According to some embodiments, the glycan or CD44 glycoform (e.g. HCELL) on the surface of the cells is stable for a length of time stored at 4° C. or less (e.g. frozen). The term “stable” as used with respect to the glycan or CD44 glycoform means that at least 99, 98, 97, 96, 95, 94, 93, 92, 91, 90, 85, 80, 75, 70, 60, 50, or 40% of the glycan or CD44 glycoform is present relative to the starting amount of glycan or CD44 glycoform. According to some embodiments, the glycan or CD44 glycoform (e.g. HCELL) is stable for at least 24, 48, 72, 96, or more, hours when stored at 4° C. or less. According to some embodiments, cell viability is maintained for a length of time (e.g. as measured by Annexin V and/or propidium iodide staining). “Viability is maintained” as used herein means that at least 99, 98, 97, 96, 95, 94, 93, 92, 91, 90, 85, 80, 75, 70, 60, 50, or 40% of the cells are alive as measured by Annexin V and/or propidium iodide staining. According to some embodiments, cell viability is maintained for at least 24, 48, 72, 96, or more, hours when stored at 4° C. or less. According to some embodiments, there is no statistically significant change in the amount of glycan or CD44 glycoform (e.g. HCELL) after at least 24, 48, 72, 96, or more, hours after contacting with a glycosyltranferase (e.g. a fucosyltransferase).

According to some embodiments, the present disclosure provides a process for producing GMP-grade exofucosylated hMSCs comprising the steps of (a) providing hMSCs a culture medium comprising a non-xenogeneic supplement; (b) expanding the hMSCs in the culture medium; and (c) contacting the hMSCs with a glycosyltransferase.

According to some embodiments, the present disclosure provides a method of treating a subject in need thereof with GMP-grade exofucosylated human mesenchymal stem cells (hMSCs) comprising administering to the subject a therapeutically effective amount of GMP-grade exofucosylated hMSCs.

According to some embodiments, the present disclosure provides a non-xenogeneic system for producing GMP-grade exofucosylated human mesenchymal stem cells (hMSCs) comprising (a) hMSCs; (b) a culture medium for passaging the hMSCs comprising a non-xenogeneic supplement; and (c) a glycosyltransferase selected from the group consisting of α(1,3)-fucosyltransferase III, IV, V, VI, VII, IX or a combination thereof.

In some embodiments, the glycosyltransferase is effective to enforce expression of one or more of Le^{x}, sLe^{x}, VIM-2, and Difucosyl sLe^{X}. In some embodiments, the supplement, such as a non-xenogeneic supplement, is effective to increase expression of a CD44 glycoform comprising Type 2 lactosamines to provide an increased number of substrates for the formation of one or more of Le^{x}, sLe^{x}, VIM-2, and Difucosyl sLe^{x} by contact with a fucosyltransferase. In some embodiments, the supplement is human platelet lysate (HPL). In some embodiments, the culture medium comprises 2% to 10% HPL v/v. In some embodiments, the culture medium comprises 5% HPL v/v. In some embodiments, the supplement is effective to increase or maintain expression of sialylated Type 2 lactosamines and, after contacting with a fucosyltransferase, the resulting fucosylated lactosaminyl glycans (e.g. HCELL) are stable for at least 48 hours at 4° C. or less. In some embodiments, the non-xenogeneic supplement is effective to maintain viability of the hMSCs for at least 48 hours at 4° C. or less.

Methods Used to Contact a Cell with a Fucosyltransferase to Enforce a Cell Surface Fucosylated Lactosaminyl Glycan

In some embodiments, the cells are contacted with the desired fucosyltransferase via exofucosyltation. For example, U.S. Pat. Nos. 7,875,585 and 8,084,236, provide compositions and methods for ex vivo modification of cell surface glycans on a viable cell, which may be used to enforce a pattern of cell surface fucosylated lactosaminyl glycans on a cell. In some embodiments, the compositions include a purified glycosyltransferase polypeptide and a physiologically acceptable solution, for use together with appropriate donor nucleotide sugars in reaction buffers and reaction conditions specifically formulated to retain cell viability. In some embodiments, the physiologically acceptable solution is free or substantially free of divalent metal co-factors, to such extent that cell viability is not compromised. In these and other embodiments, the composition is also free or substantially free of stabilizer compounds such as for example, glycerol, again, to such extent that cell viability is not compromised. Glycosyltransferases include for example, fucosyltransferase. In one embodiment, the fucosyltransferase is an α(1,3)-fucosyltransferase such as an α(1,3)-fucosyltransferase III, α(1,3)-fucosyltransferase IV, an α(1,3)-fucosyltransferase V, an α(1,3)-fucosyltransferase VI, an α(1,3)-fucosyltransferase VII or an α(1,3)-fucosyltransferase IX. In some embodiments, an additional glycosyltransferase and/or glycosidase is used to enforce the pertinent acceptor lactosaminyl glycan, upon which a fucosyltransferase could then add a fucose moiety. The glycosyltransferases and glyocosidases capable of forming lactosaminyl glycans (upon with fucose can be added by fucosyltransferase) are well known in the art. In some embodiments, α(2,3)-sialyltransferases such as ST3GalIII, ST3GalIV, and ST3GalVI, can be used to convert unsialylated (i.e., “neutral”) terminal Type 2 lactosaminyl glycans into α(2,3)-sialylated Type 2 lactosaminyl glycans, which could then be fucosylated by the fucosyltransferase(s) to create pertinent sialofucosylated lactosaminyl glycans. In some embodiments, a sialidase can be used (e.g., an α(2,3)-sialidase, or an α(2,3/2,6/2,8)-sialidase (such as sialidase from Vibrio cholerae (e.g. 0.1 U/ml; Roche)) to cleave terminal α(2,3)-sialic acid and/or terminal α(2,3)-linked, α(2,6)-linked or α(2,8)-linked sialic acid(s) off of sialylated type 2 lactosamines, thereby creating “neutral” type 2 lactosamine termini; these termini could then be fucosylated to create Le^{X}, or, in the case of (originally) α(2,6)-linked or α(2,8)-linked lactosamines, desialylated by sialidase treatment and then resialylated by α(2,3)-sialyltransferases to create α(2,3)-sialylated Type 2 lactosaminyl glycans, which could then be fucosylated by the fucosyltransferase(s) to create pertinent sialofucosylated lactosaminyl glycans (e.g., sLe^{X}). In some embodiments, a hexosaminidase may be used to cleave N-acetylgalactosamine from the Sda antigen (GalNAc-β(1,4)-[Neu5Ac-α(2,3)]-Gal-β(1,4)-GlcNAc-R) to render substrate α(2,3)-sialylated Type 2 lactosaminyl glycans, which could then be fucosylated by the fucosyltransferase(s) to create pertinent sialofucosylated lactosaminyl glycans. In some embodiments, the contacting of the combination of fucosyltransferase and addition glycosyltrasferase/glycosidase occurs simultaneously or sequentially.

According to some embodiments the human or mammalian cells may be contacted with a desired fucosyltransferase by transfecting a DNA or RNA nucleotide sequence encoding the desired fucosyltransferase into the cell. According to some embodiments, modified RNA (modRNA) encoding the relevant α(1,3)-FT transcripts is used to enforce the desired pattern of fucosylated lactosaminyl glycans. In some embodiments, the transfected nucleotide sequence encodes a full length or partial peptide sequence of the desired fucosyltransferase. In some embodiments, the nucleotide sequence encodes a naturally existing isoform of a fucosyltransferase.

According to some embodiments, the cells may be contacted with the desired fucosyltransferase by transfecting a recombinant DNA or RNA molecule. As used herein, the term “recombinant DNA or RNA” means a DNA or RNA molecule formed through recombination methods to splice fragments of DNA or RNA from a different source or from different parts of the same source. In some embodiments the recombinant DNA may comprise a plasmid vector, which controls expression of the DNA in the cell. Proteins, such as enzymes, encoded by recombinant DNA or RNA are recombinant proteins.

In some embodiments, glycans are modified on the surface of a cell by contacting a population of cells with one or more glycosyltransferase compositions described above. In some embodiments, the cells are contacted with the glycosyltransferase composition together with appropriate nucleotide sugar donor (e.g., GDP-fucose, CMP-sialic acid) under conditions in which the glycosyltransferase has enzymatic activity. Glycan modification according to this method results in cells that have at least 70%, 75%, 80%, 85%, 90%, 95%, 96%, 97%, 98%, 99% or more viability at 24 hours or more after treatment. In one embodiment, for example, the cells have at least 70% viability at 48 hours after treatment. In one such embodiment, for example, the cells have at least 75% viability at 48 hours after treatment. In one embodiment, for example, the cells have at least 80% viability at 48 hours after treatment. In addition, the phenotype of the cells (other than the glycan modification) is preferably preserved after treatment. By preserved phenotype it is meant the cell maintains its native function and/or activity. For example, if the cell is a stem cell it retains its potency, i.e., its relevant totipotency or pluripotency or multipotency or unipotency, as would be characteristic of that particular stem cell type.

According to some embodiments, glycosyltransferases are contacted with cells in the absence of divalent metal co-factors (e.g. divalent cations such as manganese, magnesium, calcium, zinc, cobalt or nickel) and stabilizers such as glycerol. In some embodiments, a purified glycosyltransferase polypeptide and a physiologically acceptable solution free of divalent metal co-factors is used to enforce a desired glycosylation pattern. The composition is free of stabilizer compounds such as for example, glycerol, or the composition contains stabilizers at levels that do not affect cell viability. Glycosyltransferase contacted with cell in the absence of divalent metal cofactors include for example, α(1,3)-fucosyltransferase such as an α 1,3 fucosyltransferase III, α 1,3 fucosyltransferase IV, an α 1,3 fucosyltransferase VI, an α 1,3 fucosyltransferase VII or an α 1,3 fucosyltransferase IX). In some embodiments, the composition further includes a sugar donor suitable for the specific glycosyltransferase. For example, when the glycosyltransferase is a fucosyltransferase, the donor is GDP-fucose. According to some embodiments, the glycosyltransferase is biologically active. By biologically active means that the glycosyltransferase is capable of transferring a sugar molecule from a donor to acceptor. For example, the glycosyltransferase is capable of transferring 0.1, 0.2, 0.3, 0.4, 0.5, 1.0, 1.5, 2.0, 2.5, 5, 10 or more moles of sugar per minute at pH 6.5 at 37° C. In some embodiments, the contacting of a glycosyltranferase with a cell occurs in a physiologically acceptable solution, which is any solution that does not cause cell damage, e.g. death. For example, the viability of the cell is at least 80%, 85%, 90%, 95%, 96%, 97%, 98%, 99% or more after treatment with the compositions of the invention. Suitable physiologically acceptable solutions include, for example, Hank's Balanced Salt Solution (HBSS), Dulbecco's Modified Eagle Medium (DMEM), a Good's buffer (see 104; 105) such as a HEPES buffer, a 2-Morpholinoethanesulfonic acid (MES) buffer, or phosphate buffered saline (PBS).

### Cell Types

Any type of cell can be used in the methods described herein, such as human or non-human. For example, according to some embodiments the cell is a somatic human cell such as an epithelial cell (e.g., a skin cell), a hepatocyte (e.g. a primary hepatocyte), a neuronal cell (e.g. a primary neuronal cell), a myoblast (e.g. a primary myoblast), or a leukocyte. The cell could be a human tissue progenitor cell or a stem cell (e.g., a mesenchymal stem cell). In some embodiments, the cell type includes, but is not limited to, embryonic stem cells, adult stem cells, induced pluripotent stem cells, blood progenitor cells, tissue progenitor cells, epithelial, endothelial, neuronal, adipose, cardiac, skeletal muscle, fibroblast, immune cells (for example, dendritic cells, monocytes, macrophages, granulocytes, lymphocyte-type leukocytes (e.g., a lymphocyte such as a B-lymphocyte, a T-lymphocyte, or a subset of T-lymphocytes, such as regulatory lymphocyte (e.g., CD4⁺/CD25⁺/FOXP3⁺ cells, Breg cells, etc.), a naive T cell, a central memory T cell, an effector memory T cell, an effector T cell, NK cells, etc.), hepatic, splenic, lung, circulating blood cells, platelets, reproductive cells, gastrointestinal cells, renal cells, bone marrow cells, cardiac cells, endothelial cells, endocrine cells, skin cells, muscle cells, neuronal cells, and pancreatic cells. The cell can be an umbilical cord stem cell, an embryonic stem cell, or a cell isolated from any tissue (such as a primary cell) including, but not limited to brain, liver, lung, gut, stomach, fat, muscle, testes, uterus, ovary, skin, spleen, endocrine organ and bone, and the like. The cell can be culture-expanded and/or modified in vitro by introduction of any nucleic acid sequence encoding a protein of interest. The cell can be derived from a tissue progenitor cell or a stem cell or a somatic cell (e.g., a monocyte-derived dendritic cell).

Where the cell is maintained under in vitro conditions, conventional tissue culture conditions and methods can be used, and are known to those of skill in the art. Isolation and culture methods, and cell expansion methods, for various cells are well within the knowledge of one skilled in the art. Moreover, various cells that contain nucleic acid encoding desired protein products are also incorporated (e.g., CAR-T cells, nucleic acid modified cells, gene-modified cells, RNA-modified cells, etc.).

In addition, both heterogeneous and homogeneous cell populations are contemplated for use with the methods and compositions described herein. In addition, aggregates of cells, cells attached to or encapsulated within particles, cells within injectable delivery vehicles such as hydrogels, and cells attached to transplantable substrates (including scaffolds) or applied into tissue(s) that harbors scaffolds/transplantable substrates are contemplated for use with the methods and compositions described herein. Moreover, cells may be used in combination with tissue proliferative/enhancing agents and/or anti-inflammatory agents (e.g., growth factors, cytokines, prostaglandins, trophic agents, Resolvins, NSAIDS, steroids, etc.)

### Administration of Cell Populations Described Herein

Administration of cell populations described herein for therapeutic indications can be achieved in a variety of ways, in each case as clinically warranted/indicated, using a variety of anatomic access devices, a variety of administration devices, and a variety of anatomic approaches, with or without support of anatomic imaging modalities (e.g., radiologic, MRI, ultrasound, etc.) or mapping technologies (e.g., epiphysiologic mapping procedures, electromyographic procedures, electrodiagnostic procedures, etc.). Cells can be administered systemically, via either peripheral vascular access (e.g., intravenous placement, peripheral venous access devices, etc.) or central vascular access (e.g., central venous catheter/devices, arterial access devices/approaches, etc.). Cells can be delivered intravascularly into anatomic feeder vessels of an intended tissue site using catheter-based approaches or other vascular access devices (e.g., cardiac catheterization, etc.) that will deliver a vascular bolus of cells to the intended site. Cells can be introduced into the spinal canal and/or intraventricularly intrathecally, into the subarachnoid space to distribute within cerebrospinal fluid and/or within the ventricles). Cells can be administered directly into body cavities or anatomic compartments by either catheter-based approaches or direct injection (e.g., intraperitoneal, intrapleural, intrapericardial, intravesicularly (e.g., into bladder, into gall bladder, into bone marrow, into biliary system (including biliary duct and pancreatic duct network), intraurethrally, via renal pelvis/intraureteral approaches, intravaginally, etc.)). Cells can be introduced by direct local tissue injection, using either intravascular approaches (e.g., endomyocardial injection), or percutaneous approaches, or via surgical exposure/approaches to the tissue, or via laparoscopic/thoracoscopic/endoscopic/colonoscopic approaches, or directly into anatomically accessible tissue sites and/or guided by imaging techniques (e.g., intra-articular, into spinal discs and other cartilage, into bones, into muscles, into skin, into connective tissues, and into relevant tissues/organs such as central nervous system, peripheral nervous system, heart, liver, kidneys, spleen, etc.). Cells can also be placed directly onto relevant tissue surfaces/sites (e.g., placement onto tissue directly, onto ulcers, onto burn surfaces, onto serosal or mucosal surfaces, onto epicardium, etc.). Cells can also administered into tissue or structural support devices (e.g., tissue scaffold devices and/or embedded within scaffolds placed into tissues, etc.), and/or administered in gels, and/or administered together with enhancing agents (e.g., admixed with supportive cells, cytokines, growth factors, resolvins, anti-inflammatory agents, etc.).

According to some embodiments, the cell population is administered to the subject with an enforced expression of glycosylation. According to some embodiments, the enforced glycosylation on the surface of administered cells will aid in revascularization, in host defense (e.g., against infection or cancer) and/or in tissue repair/regeneration and/or mediate immunomodulatory processes that will dampen inflammation and/or prevent inflammation. According to some embodiments, the enforced glycosylation pattern guides delivery of intravascularly administered cells to sites of inflammation by mediating binding of blood-borne cells to vascular E-selectin expressed on endothelial cells at sites of inflammation. Moreover, whether cells are administered systemically, intravascularly, into the spinal canal and/or intraventricularly intrathecally, into the subarachnoid space to distribute within cerebrospinal fluid), directly into body cavities or compartments, by direct local tissue injection, or by placement onto relevant tissue surfaces/sites, the enforced expression of ligands for E-selectin and/or L-selectin on administered cells promotes lodgment of cells within the affected tissue milieu, in apposition to cells bearing E-selectin (i.e., endothelial cells) and/or L-selectin (i.e., leukocytes), respectively, within the target site. Thus, the spatial distribution and localization of administered cells within the target tissue is modulated by the enforced glycosylation on administered cells.

Particularly, the colonization of a desired cell type at a site of inflammation occurs as a result of the enforced glycosylation on the administered cells, such that the administered cells have augmented binding to E-selectin, thereby promoting the systemic delivery of the desired cells and/or the lodgment of cells when injected directly into the affected site. For example, the enforced glycosylation of E-selectin ligands (e.g., HCELL) is advantageously capable of anchoring directly injected cells within E-selectin-expressing vessels at sites of inflammation, tissue injury, or cancer. Thus, the present methods augment efficiency in the delivery of relevant cells at or to a site of inflammation, tissue injury, or cancer, including, for example, the capacity to deliver tissue-reparative stem cells, to deliver immunomodulatory cells (e.g., mesenchymal stem cells, T-regulatory cells, B-regulatory cells, NK-cells, dendritic cells, etc.), and the capacity to deliver immune effector cells to combat the inciting inflammatory process or cancer (e.g., in the case of infection or malignancy, delivery of pathogen-specific immune effector T cells or cancer-specific cytotoxic T cells or NK cells, respectively); such immunologic cells (regulatory T-cells, NK cells, cytotoxic T-cells, dendritic cells, etc.) may be antigen-pulsed, tumor cell pulsed, virus pulsed, and other means to create antigen specificity (e.g., genetic engineering of antigen receptors (e.g., CAR-T cells) or other forms of creation of antigen-specific cells). Similarly, the enforced glycosylation of L-selectin ligands (e.g., HCELL) is advantageously capable of anchoring directly injected cells within L-selectin-expressing cells infiltrating sites of inflammation, tissue injury, or cancer.

According to some embodiments, the enforced glycosylation on the cell surface will drive vascular homing of cells to any site where E-selectin is expressed. In various embodiments, the cell population comprises Le^{x}, sLe^{x}, VIM-2, and/or Di-Fuc-sLe^{X}. For example, since CD44 is a ubiquitously expressed cell membrane protein and is displayed on stem/progenitor cell populations of both “adult” and embryonic types, the capacity to modify glycosylation of this protein by ex vivo glycan engineering to create the HCELL (CD44 glycoform) phenotype will drive migration of injected (e.g., intravascularly) (adoptively transferred) cells in vivo to marrow or to any tissue/organ site where E-selectin is expressed. Thus, the modified cells can be used in therapeutic settings to achieve targeted cell migration in a variety of physiologic and pathologic processes, including, for example, bone diseases, immune diseases, infectious diseases, and cancer therapeutics, to name just a few conditions.

In some embodiments the disease, disorder, or medical condition having associated inflammation can be treated using the instant methods even in the absence of differentiation of the cell population in the subject. That is, there are trophic effects of administered cells at the site of inflammation without persistent engraftment and/or repopulation of the administered cells, irrespective of the type of tissue involved. These trophic effects include release of cytokines/growth factors that promote revascularization (e.g., VEGF), that promote tissue repair (e.g., TGF-β), that are immunomodulatory (e.g., IL-10), that stimulate growth/proliferation of tissue-resident progenitors (e.g., SCF, LIF, etc) and many other tissue-reparative processes (e.g., mitochondria delivery to cells). In addition, administered cells (e.g., Tregs, MSCs, dendritic cells, etc.) may have potent immunomodulatory properties, including direct suppression of activated lymphocytes (e.g., via expression of PDL-1).

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods of the present invention. These examples are illustrative only and are not intended to limit the scope of the invention in any way.

### Example 1—Methods

Isolation, Culture and Expansion of Human hMSCs

This research effort was approved by the Institutional Review Boards of the University Hospital Virgen de la Arrixaca (Murcia, Spain) and of Partners Healthcare System (Massachusetts General Hospital/Brigham & Women's Hospital, Boston, Mass.). As needed, written informed consent was obtained from donors as per Helsinki Declaration guidelines. To obtain HPL, discarded platelet transfusion bags were frozen at −80° C. then thawed at 37° C. Lysates were centrifuged at 900 g for 30 min, and the supernatants were then collected, aliquoted and stored at −20° C.

For laboratory-scale experiments comparing the two different culture and plate-lifting conditions (i.e., the use of FBS/porcine trypsin (Gibco, Grand Island, N.Y.) and the use of HPL/TrypLE Select (Gibco) reagents), hMSCs were obtained from remnant cells within collection bags and filters of BM harvests of normal donors (for hematopoietic stem cell (HSC) transplant) at the Massachusetts General Hospital under protocols approved by the human Experimentation and Ethics Committee of Partners Healthcare. Nucleated cells (NC) were flushed from bags and filters using calcium-free Dulbecco's PBS (DPBS) (Gibco), and BM mononuclear cells (BMMCs) were isolated by density gradient centrifugation over Histopaque-1077 (Sigma-Aldrich, St Louis, Mo.). BMMCs were then plated in 175 cm2 culture flasks at 160,000 cells/cm2 in low glucose DMEM (Gibco Invitrogen Corporation, Grand Island, N.Y., USA) supplemented either with 10% FBS or with 5% HPL, 1% penicillin streptomycin, 2 U/ml heparin and incubated at 37° C. in a humidified atmosphere containing 5% CO2 and 21% O2. Cells were grown to maximal 70% confluency at each passage. The cellular expansion growth rate of hMSCs was evaluated by counting the cells at each passage, and expressed in terms of cumulative population doubling (CPD) using the formula log N/log 2, where N is the cell number of the confluent monolayer divided by the initial number of cells seeded. All studies were performed on cells propagated within culture passage 3-5. After the third passage, hMSCs were harvested and characterized according to the International Society for Cellular Therapy (ISCT) criteria (37).

Sterility testing was performed in the BacT/ALERTR 3D system (BioMerieux, France) using 40 ml bottles of BacT/ALERTR SA (for aerobes) and BacT/ALERTR SN (for anaerobes) media. The respective BacT bottles (supplied by BioMerieux (France)) were inoculated with the samples and incubated at 32.5±2.5° C. for 14 days. Automatic readings were taken every 10 minutes. Samples were tested for Endotoxin using the Endosafe® Portable Test System (PTS™; Charles River Labs, Wilmington, Mass.), a chromogenic Limulus Amebocyte Lysate (LAL) endotoxin detection system which provides quantitative results in 15 minutes; we used 0.5-0.005 endotoxin units/mL (EU/mL) sensitivity cartridges. Mycoplasma testing was performed by PCR using Venor®GeM OneStep Mycoplasma detection kit (Minerva Labs, Berlin, DE).

Design and Validation of a Consistent and Reproducible Manufacturing Process to Produce Clinical-Grade Exofucosylated hMSCs

On the basis of the acquired experience with laboratory-scale batches, we created standard operating procedures and batch record sheets to validate an hMSC fucosylation protocol in compliance with GMP standards. We established specifications and test methods for quality control using multiple specimens of hMSCs expanded from BM harvest bags/filters. From these cultures, validation protocols were also approved before starting the manufacture of the GMP-required 3 validation batches. For this purpose, we used 4 BM samples obtained from 3 volunteer donors. The first lot of hMSCs was obtained from 120 ml of BM from a healthy 44-year-old woman; the second, by thawing the replacement cell bank from this first lot. The third and fourth samples were obtained from 60 ml of BM from each of two osteoporotic women, aged 64- and 74-years, as we aimed to test the ability to culture-expand hMSCs obtained from osteoporotic bones. BMMCs were isolated using the Sepax Cell Processing System (Biosafe, Eysins, Switzerland), following the manufacturer's instructions. hMSCs were expanded using 2-layer or 4-layer Nunc Cell Factory Systems (Thermo Fisher Scientific, Waltham, Mass.).

As these validations were focused on clinical application, exofucosylation was performed using two different α(1,3)-fucosyltransferases, FTVI and FTVII. Hematopoietic cell lines that natively express sLe^{X} or that do not express sLe^{X} were used as controls for exofucosylation efficiency: typically, cell line KG1a (sLe^{X}+) served as positive control to confirm sLeX detection experimentally by flow cytometry and western blot, and RPMI 8402 (which expresses sialylated type 2 lactosamines, but does not natively display sLe^{X}) was used to assess the extent of enforced expression of sLe^{X} following exofucosylation. Other cell lines were used as controls for c-Myc expression (Daudi, DG-75, Jurkat, K-562, HeLa, and Namalwa). All cell lines were obtained from ATCC (Manassas, Va.), and expanded in RPMI 1640 medium supplemented with 10% FBS and 1% penicillin-streptomycin.

Glycoengineering of sLe^{X} Expression on hMSCs

For α(1,3)-exofucosylation of hMSCs, all reagents utilized were sterile, and all reactions were performed under sterile conditions with endotoxin levels below that of sterile water USP for injection (<0.25 EU/ml). To this end, 1×106 hMSCs were buffer-treated (without enzyme) or treated either with 1 ug of FTVI (Warrior Therapeutics, Sudbury, Mass.) or 2 ug FTVII (Warrior Therapeutics, Sudbury, MA) in 50 μL of HBSS without Ca2+ or Mg2+ (Gibco), containing 10 mM HEPES, 0.1% human serum albumin (RMBIO, Missoula, Mont.), and 1 mM GDP-fucose (Carbosynth, Compton, UK), at 37° C. for 1 hour with gentle shaking. The cells were then collected by centrifugation and washed ×2 with DPBS (Gibco) (24,38).

### Western Blot Analysis

To obtain cell lysates, cell lines or hMSCs were suspended in 50 mM Tris-Cl (pH 7.4), 150 mM NaCl, 20 μg/ml PMSF, 0.02% NaN3 and Protease Inhibitor Cocktail (Roche, Nutley, N.J.), sonicated, and then solubilized in 2% Nonidet P-40 (NP-40). Proteins in lysates were quantified by Bradford assay, and all lysates loaded in gel lanes were normalized for protein content, then resolved by 7.5% sodium dodecyl sulfate polyacrylamide gel electrophoresis (SDS-PAGE)(Bio-Rad, Hercules, Calif.). Gels were transferred to Polyscreen polyvinylidene difluoride (PVDF) membranes (Bio-Rad) and blocked with 10% non-fat dry milk and 0.1% Tween-20 in TBS (TBS-T). For assessment of E-selectin ligands, as well as detection of sLe^{X} and CD44, membranes were incubated with primary and secondary antibodies as described previously (24).

Immunophenotyping of hMSCs after FTVI and FTVII Exofucosylation

The immunophenotypic characterization of the hMSCs was performed according to the recommendations of the ISCT, using the hMSCs Phenotyping Kit (Miltenyi Biotec, Bergisch Gladbach, DE). Briefly, 1×105 cells were incubated for 20 min at 4° C. using labelling buffer (DPBS with 1% FBS and 2 mM EDTA) containing the hMSC Phenotyping Cocktail (CD14-PerCP, CD2O-PerCP, CD34-PerCP, CD45-PerCP, CD73-APC, CD90 FITC, CD106 PE) or the Isotype Control Cocktail (Mouse IgG1-FITC, mouse IgG1-PE, mouse IgG1-APC, mouse IgG1-PerCP, mouse IgG2a-FITC). Data were collected using a Navios flow cytometer (Beckman Coulter, Miami, Fla.), and analyzed with Kaluza 1.2 program (Beckman Coulter.) Apoptosis was determined by dual labelling with annexin V and propidium iodide (Immunostep, Salamanca, Spain).

Flow Cytometry to Assess HECA-452, CSLEX-1, CD15 and CD44 Expression Before and after FTVI and FTVII Exofucosylation

For flow cytometry, all mAb staining was performed for 30 minutes at 4° C. For detection of sLe^{X}, staining used FITC-conjugated anti-human/mouse clone HECA-452 (Rat IgM; Southern Biotech) and, also, FITC-conjugated anti-human CD15s mAb (clone CSLEX-1; IgM) (Southern Biotech). For detection of CD15 (Le^{x}), staining used FITC-conjugated anti-human CD15 (clone HI98; IgM) (Southern Biotech). CD44 staining used anti-human CD44-PE mouse mAb (clone G44-26; IgG2) (BD Biosciences).

### Stamper-Woodruff Cell Binding Assay to Assess L-Selectin Binding Activity of HCELL

HCELL binds both E-selectin and L-selectin. To assess L-selectin ligand activity of exofucosylated hMSCs, we utilized a modification of the Stamper-Woodruff assay as previously described (15). Briefly, untreated and α(1,3)-exofucosylated hMSCs were cytocentrifuged onto glass slides (Shandon Cytospin3, Thermo Fisher Scientific), fixed in 3% glutaraldehyde, and blocked in 0.2 M lysine. Human peripheral blood mononuclear cells (PBMC) were isolated by Ficoll Histopaque density gradient centrifugation of whole blood, and suspended at 1×107 cells/ml in RPMI 1640 medium supplemented with 2% FBS. The PBMC suspensions were then overlaid onto fixed slides containing hMSCs, and slides were placed on an orbital shaker at 80 rpm for 30 min at 4° C., following which slides were washed with PBS and fixed in 3% glutaraldehyde. PBMC binding to hMSCs was observed by phase contrast with an inverted light microscope (39). Controls for binding specificity included assays in the presence of function-blocking anti-L-selectin mAb (LAM1-3; Biolegend) and assays performed in presence of EDTA.

Assessment of hMSCs Differentiation Capacity Following Exofucosylation

To analyze differentiation potential of buffer-treated and exofucosylated hMSCs, cells were subjected to osteogenic, adipogenic and chondrogenic differentiation using NH OsteoDiff Medium, NH AdipoDiff Medium and NH ChondroDiff Medium (Miltenyi biotec) respectively, as per manufacturer's instructions. Medium was changed every 3-4 days. Parallel control cultures were performed using standard DMEM supplemented with 5% HPL. For assessment of osteogenic and adipogenic differentiation, cells were fixed with cold 70% methanol for 5 min. Osteogenic differentiation was evaluated using two indicators: alkaline phosphatase (AP) activity and the presence of calcium deposits. AP activity was detected as a dark purple staining after incubation with SigmaFast BCIP/NBT (Sigma-Aldrich) for 10 min at RT. Calcium deposits were detected by orange staining after incubation with Alizarin Red for 30 min at RT. Adipogenic differentiation was detected by the presence of red-stained cytoplasmic vacuoles observed after incubation with Oil Red for 20 min at RT.

Cytogenetic Analysis, Real-Time Quantitative PCR Analysis (qRT-PCR) of c-Myc, Microarray Analysis, and Receptor Tyrosine Kinase (RTK) Phosphorylation Analysis.

For all GMP validation cultures, hMSCs were α(1,3)-fucosylated using either FTVI or FTVII, and subsequently analyzed for genetic stability, c-Myc expression, gene expression profile (by microarray), and RTK phosphorylation status. Genetic stability of the validation batches was studied after the second or third passage (with hMSCs at 60%-70% confluence) by conventional G-banding karyotype analysis. The expression levels of c-Myc mRNA were measured in untreated (n=13), buffer-treated (n=8), FTVI-treated (n=4) and FTVII-treated (n=4) hMSCs by qRT-PCR. To this end, total RNA was obtained from 1×106 hMSCs using the miRNeasy Mini Kit (Qiagen, Hilden, DE). RNA samples were quantitated in a NanoDrop 2000 (Thermo Fisher Scientific). RNA quality was examined in an Agilent 2100 Bioanalyzer (Agilent Technologies, Palo Alto, Calif.). In most samples, the R.I.N. (RNA Integrity Number) was equal to 10. First-strand cDNA was synthesized from 0.5 ug total RNA using the iScript™ cDNA synthesis kit (Bio-Rad). Predesigned QuantiTect primers (Qiagen) for MYC (QT00035406) and GAPDH (QT01192646), as a gene reference, were used. qRT-PCR was performed in an ABI Prism 7000 Sequence Detection System with SYBR Green Master mix (Takara Bio, Mountain View, Calif.). The relative fold differences in transcript expression were calculated by adapting the 2-ΔΔCt as described elsewhere (40).

For microarray analysis, RNA samples from buffer-treated (n=7), FTVI-treated (n=4) and FTVII-treated (n=3) hMSCs were labelled using Agilent Two Color Quick Amp Labelling and RNA Spike-In kits. Each of the samples was labelled with cyanine 5-CTP. A pooled sample composed of equimolar amounts of RNA from untreated hMSCs (n=5) was labelled with cyanine 3-CTP. The labelled RNAs were mixed together and hybridized using the Agilent Gene Expression Hybridization kit onto SurePrint G3 Human Gene Expression 8×60K v2 Microarrays, containing 58,717 probes targeting 50,599 different biological features (genes and lncRNAs). After hybridization, the microarray slides were washed and scanned in an Agilent G2565CA DNA Microarray Scanner. Images were analyzed with the Agilent Feature Extraction software to automatically generate the datasets. Log 10 ratios (test vs reference) were computed after normalization correction performed by linear and Lowess methods. Datasets were deposited at the Gene Expression Omnibus database under accession number GSE90131.

Evaluation of the relative levels of phosphorylation of 71 different human RTKs was undertaken using Human RTK Phosphorylation Antibody Array G-series 1 Glass Chips (RayBiotech, Norcross, Ga.) following the manufacturer's instructions, through a service provided by Tebu-bio (Le Perray-en-Yvelines, France). This analysis was performed in buffer-treated (n=11), FTVI-treated (n=7) and FTVII-treated (n=4) hMSCs.

### Example 2—Results

FTVI- and FTVII-Mediated α(1,3)-Fucosylation of hMSCs Cultured with Either FBS or HPL Converts Cell Surface CD44 into HCELL

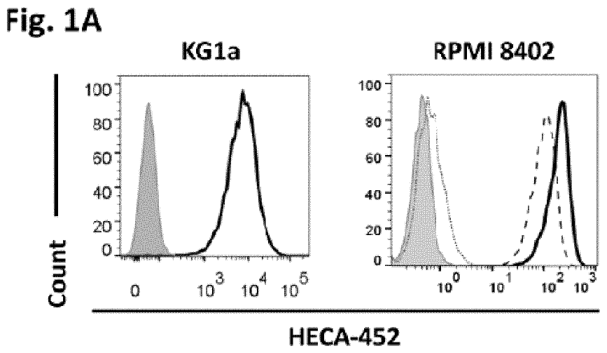

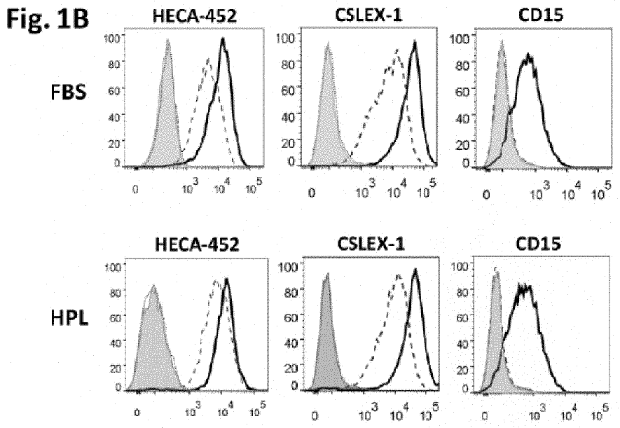

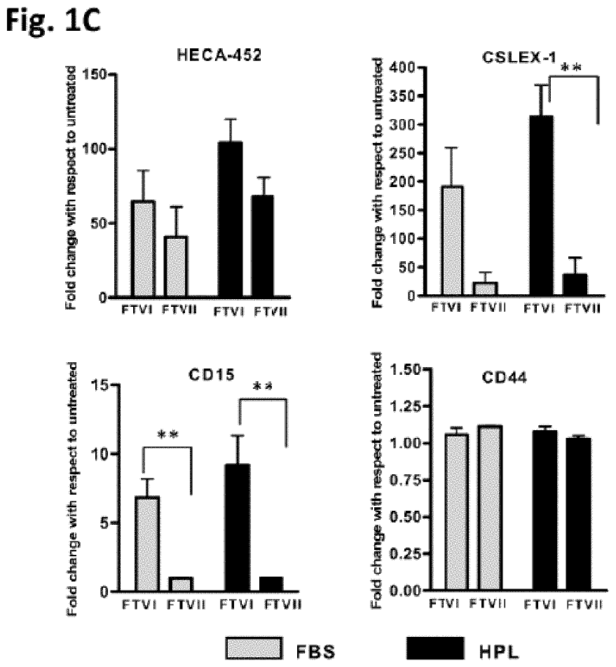

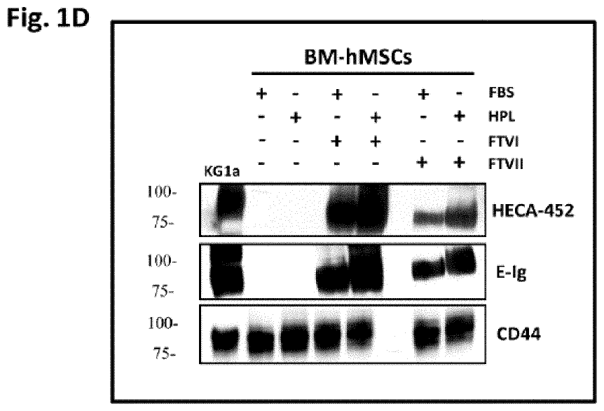

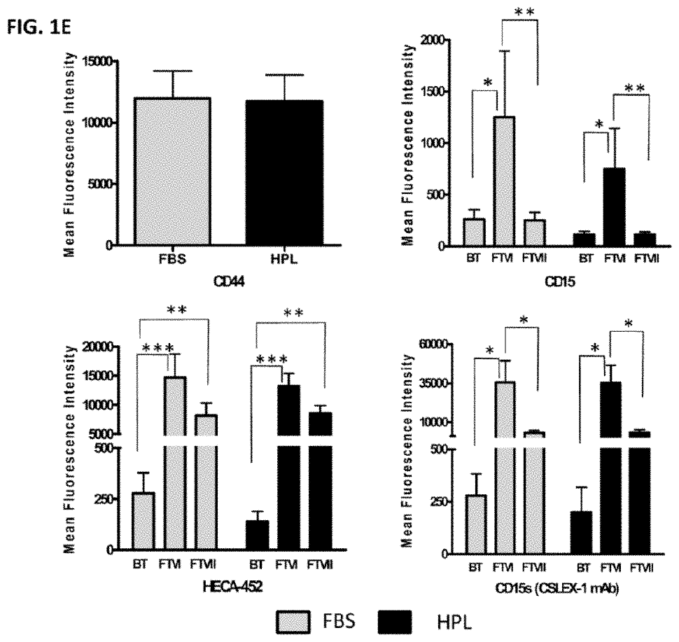

Clinical application of glycoengineered cells favors the use of reagents that are free of animal-derived components. To this end, we aimed to assess whether CD44 could be efficiently exofucosylated into HCELL on hMSCs expanded in vitro using HPL as supplement for culture medium instead of FBS. First, we studied the efficiency of exofucosylation using the human hematopoietic cell line RPMI 8402; similar to hMSCs, these cells are CD44+ and natively lack expression of sLe^{X} (as measured using the anti-sLe^{X} mAb HECA-452), and they also express a CD44 glycovariant that possesses sialylated type 2 lactosamine residues that can serve as acceptors of α(1,3)-fucosyltransferases (24-25). As such, after exofucosylation with either FTVI or FTVII, RPMI 8402 cells are HECA-452-reactive (i.e., express the sLe^{X} determinant) (FIG. 1A, right).

To assess whether hMSCs under HPL-supplemented growth conditions express glycoconjugates displaying terminal type 2 lactosaminyl glycans (both neutral (unsialylated) and sialylated lactosamines), we measured expression of LeX (CD15) and of enforced E-selectin ligands on hMSCs compared to that obtained under standard FBS-supplemented conditions. Accordingly, surface levels of sLeX were measured by flow cytometry on untreated, FTVI- and FTVII-treated hMSCs cultured either with FBS or HPL using both HECA-452 mAb and CSLEX-1 mAb as probes, and by western blot using HECA452 mAb and E-selectin-Ig chimera (E-Ig) as probes.

Treatment of hMSCs with both enzymes showed marked induction of HECA-452 reactivity by both flow cytometry and western blot (FIGS. 1B-1E). Notably, the FTVI enzyme is capable of fucosylating two glycan acceptors, unsialylated (“neutral”) type 2 lactosamine or α(2,3)-sialylated type 2 lactosamine (it can thus render the fucosylated glycans LeX and sLeX, respectively), whereas FTVII specifically fucosylates only α(2,3)-sialylated type 2 lactosamine acceptors (thereby creating sLeX) (FIG. 1B, 1C, 1E). Similar to prior results obtained with FTVI-mediated exofucosylation (25), FTVII treatment of hMSCs engendered CSLEX-1 and HECA-452 reactivity, however, significantly higher reactivity was observed after exofucosylation with FTVI; interestingly, when normalized for staining level of buffer-treated cells, higher-fold increases in FT-enforced expression of fucosylated lactosaminyl glycans—both CD15 (LeX) and CD15s (sLeX) were observed under HPL conditions than under FBS conditions (FIGS. 1B and 1C, and FIG. 1E). However, no differences in CD44 expression were observed between hMSCs cultured in HPL or FBS (FIG. 1C and FIG. 1E).

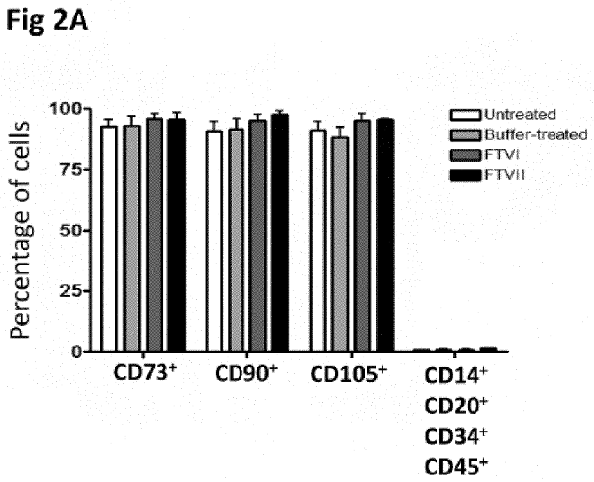

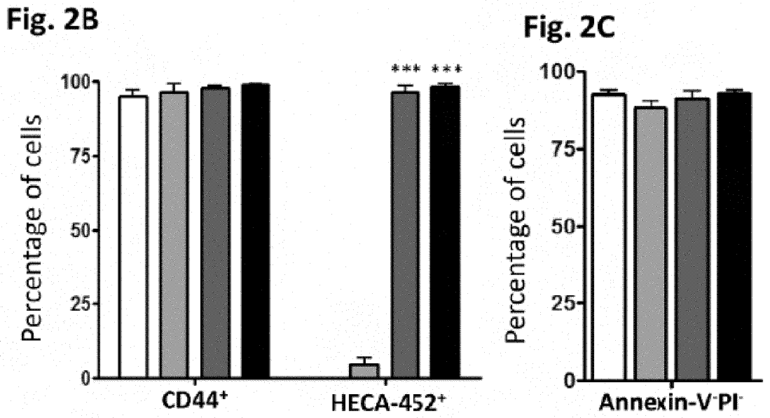

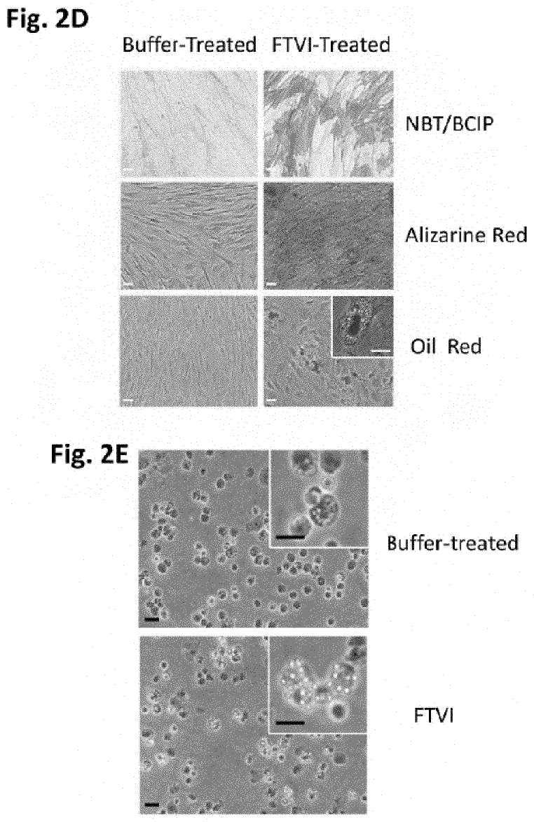

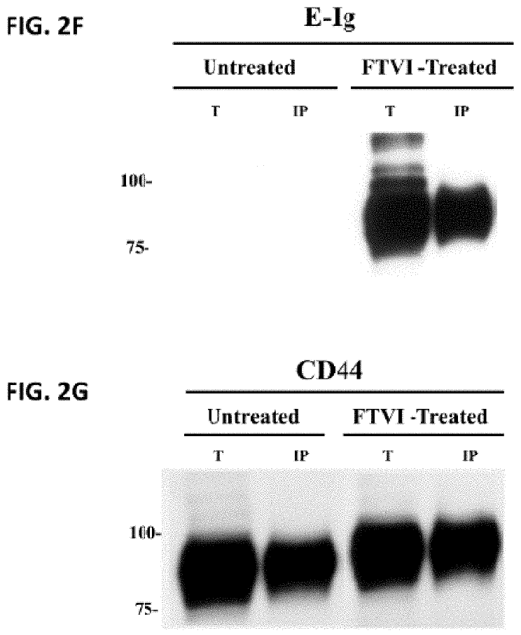

To further assess the product(s) of the exofucosylation reaction, glycoprotein extracts were analyzed by western blot using three different Abs: HECA-452 mAb (which recognizes sLeX determinants), E-selectin chimera (that recognizes E-selectin ligands), and CD44 mAb. Whole-cell extracts from KG1a and RPMI 8402 cells were used as controls. HECA-452 mAb and E-selectin-Ig chimera detected glycoproteins of molecular weight ˜90 kDa on exofucosylated hMSCs, a size consistent with that of standard CD44 on KG1a, but no bands were stained in untreated hMSCs (FIG. 1D, upper and middle panels). CD44 mAb detected glycoproteins of molecular weight ˜90 kDa in KG1a, and in untreated and FTVI-hMSCs, with no significant variations in CD44 levels among the different conditions (FIG. 1D, lower panel). CD44 immunoprecipitated from exofucosylated hMSCs stained with E-Ig on western blot, indicating that exofucosylation of sialyllactosaminyl glycans of CD44 generates sLeX, thereby endowing E-selectin binding activity (FIG. 2). Thus, CD44 is efficiently converted into HCELL by extracellular α(1,3)-fucosylation on BM-hMSCs cultured using HPL-supplementation and harnessed using TrypLE Select reagent. Those these data are similar to results using BM-hMSCs that have been propagated in FBS-supplemented media and have been detached using porcine trypsin (24,25), enforced expression of HCELL is significantly higher on cells cultured with HPL compared with FBS, without significant changes in CD44 expression. Moreover, as detected by flow cytometry, we found that FTVI induces higher HECA-452 and E-selectin-Ig reactivity on hMSCs compared with FTVII.

Design and Validation of a Consistent and Reproducible Manufacturing Process for the Production of Clinical-Grade Exofucosylated hMSCs

Based on the aforementioned results, hMSCs obtained from samples processed using the Sepax system were expanded using cell culture factories (1-2×10⁸ cells), cultured using HPL, and exofucosylated either with FVTI and FTVII. After exofucosylation, flow cytometry data indicated that hMSCs retained their immunophenotypic identity (FIG. 2A) with preservation of cell viability (FIG. 2C); hMSCs were uniformly positive for CD73, CD90, CD105, CD44 and negative for CD14, CD20, CD34 and CD45, but efficiently acquired the sLeX antigen determinant (FIG. 2B). To assess whether exofucosylation alters differentiation capacity of hMSCs under HPL culture conditions, buffer-treated and FTVI-treated hMSCs were incubated in osteogenic and adipogenic differentiation media. Exofucosylated hMSCs retained their osteogenic differentiation potential in vitro as demonstrated by presence of alkaline phosphatase activity using the NBT/BCIP chromogenic substrate and by detection of extra-cellular calcium deposits using the Alizarin Red staining. Adipogenic differentiation in NH AdipoDiff medium was shown by Oil Red staining of lipid vacuoles (FIG. 2D).

These results indicate that the culture expansion conditions chosen, and the exofucosylation protocol, all of which fulfill GMP standards for application in a clinical setting, is efficient and preserves hMSCs identity and viability. In addition, quality controls needed for the release of cell therapy batches such as sterility testing, mycoplasma detection, endotoxin test and Gram staining were performed. Those tests gave negative results in all cases (data not shown).

FTVI Exofucosylation Increases L-Selectin Ligand Activity on hMSCs

To assess the L-selectin ligand activity of HCELL engendered by α(1,3)-fucosylation of hMSCs cultured using HPL and lifted with TrypLE Select, we performed Stamper-Woodruff assays in which PBMCs (lymphocytes) were overlaid onto cytocentrifuged hMSCs. Untreated hMSCs, (which lack HECA-452 and CSLEX-1 staining) did not engage lymphocytes in Stamper-Woodruff assays, whereas FTVI-treated hMSCs showed marked L-selectin-dependent lymphocyte adhesion (FIG. 2E).

Stability of α(1,3)-Exofucosylated hMSCs Stored at RT and at 4° C.

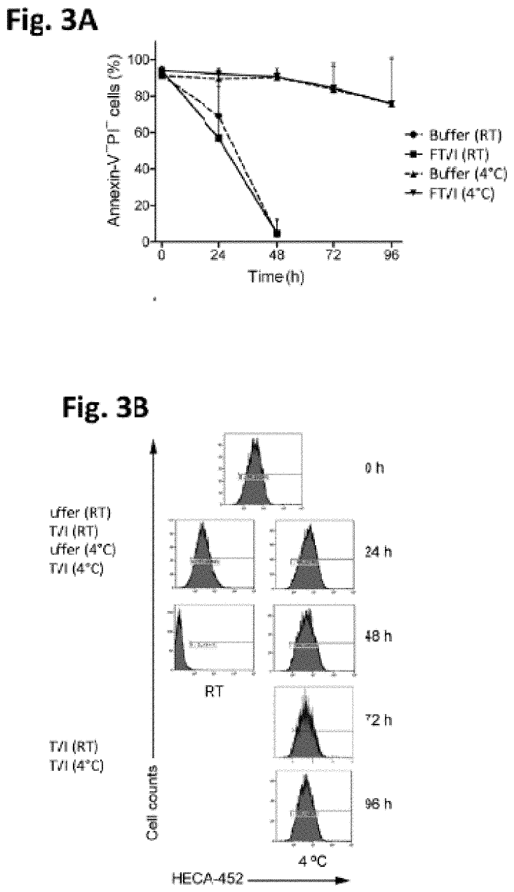

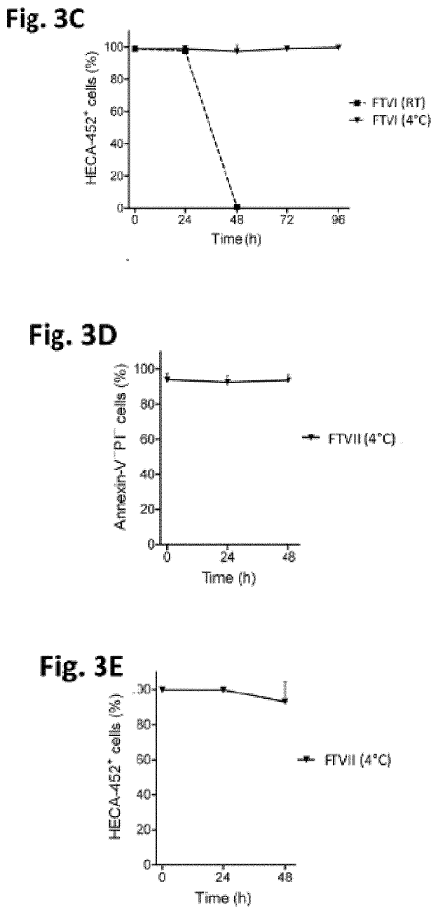

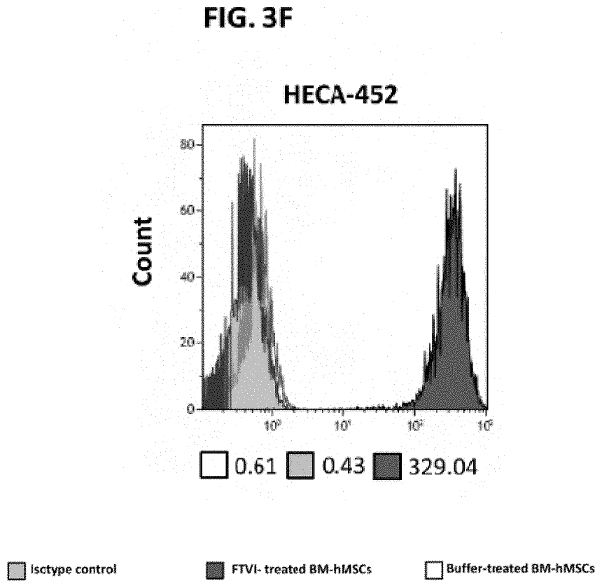

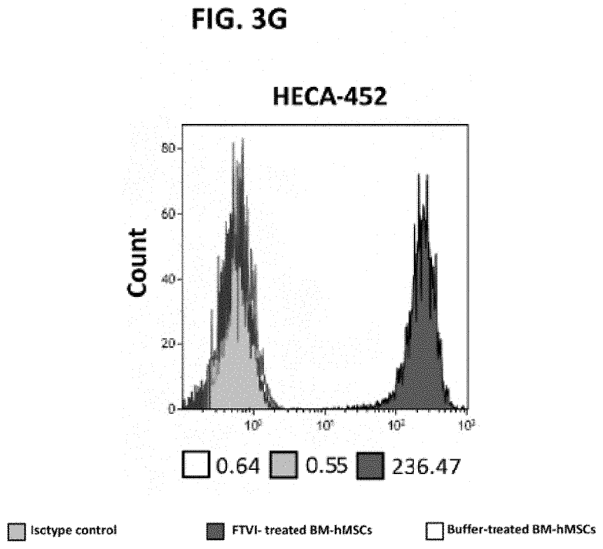

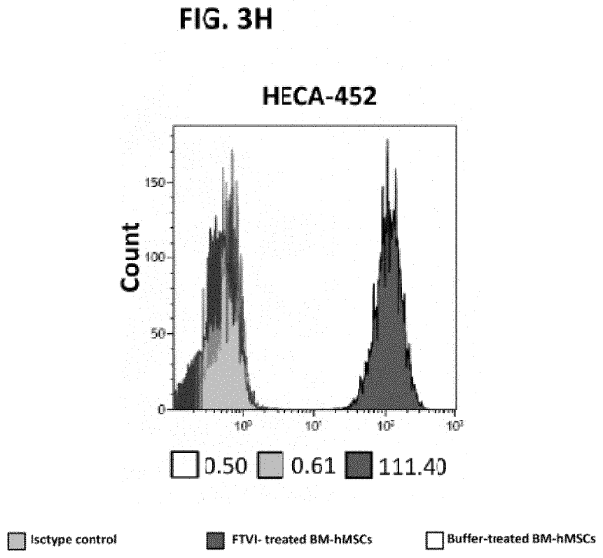

In cells cultured using HPL and lifted with TrypLE Select reagent, we assessed both cell viability and the stability of sLeX expression (HECA452-reactivity) among untreated and exofucosylated hMSCs at different time points in cells maintained at RT and at 4° C. within HPL-containing media. As measured as the percentage of annexin-V−/PI− cells, cell viability was unaffected by exofucosylation, and remained stable for at least 48 h for hMSCs stored at 4° C.; however, for both buffer-treated and FTVI-treated hMSCs, RT storage precipitously dropped viability within 48 h (FIG. 3A). Notably, for cells stored at RT, HECA452-reactivity persisted for 24 h and was lost within 48 h, whereas HECA452-reactivity remained completely stable with storage of cells at 4° C. for up to 96 h (FIGS. 3B and 3C). Based on these results, stability of FTVII-treated validation lots was assessed only with storage at 4° C. for the first 48 h after exofucosylation; similar to FTVI-treated hMSCs, both cell viability and sLeX expression were stable at 4° C. for 48 h (FIGS. 3D and 3E, respectively). In addition, we observed that HECA452-reactivity is retained following cryopreservation of exofucosylated hMSC, and sLeX expression is maintained for 24 hours after reculturing of the thawed exofucosylated hMSCs in HPL-containing media (FIG. 3).

FTVI and FTVII-Mediated α(1,3)-Exofucosylation does not Affect Karyotype and c-Myc Expression of hMSCs

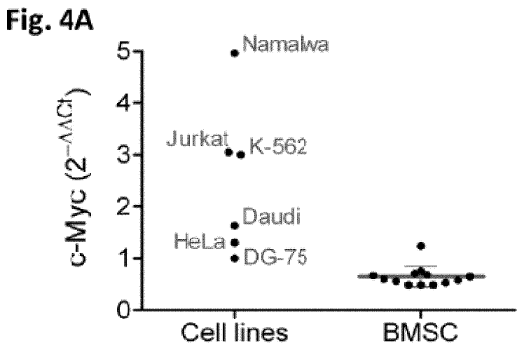

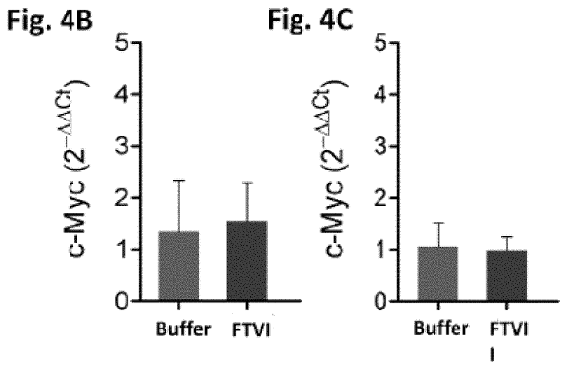

To evaluate genomic stability during the expansion phase using HPL and TrypLE Select reagent a karyotype analysis was performed. Prior to enzymatic fucosylation, as well as in all validation batches, karyotype was normal (n=4; data not shown). In addition, we measured expression by qRT-PCR of the tumor marker c-Myc in hMSCs during the expansion phase using HPL-supplemented media and lifting with TrypLE Select reagent, and following exofucosylation. To set references for this assay, a panel of 6 human cell lines was used. Among them, DG75 and Namalwa expressed the lowest (ΔCt=4.44) and highest (ΔCt=2.13) levels of c-Myc transcripts, respectively. DG75 was set as reference for c-Myc measurements (ΔΔCt=0), and hMSCs exhibited low levels of c-Myc expression (n=13, FIG. 4A), with no significant changes in levels following treatment with either FTVI (n=4, FIG. 4B) or FTVII (n=4, FIG. 4C).

Gene Expression Profile of α(1,3)-Exofucosylated hMSCs

To assess whether the GPS technology reprograms gene expression in hMSCs, gene expression microarrays following exofucosylation were performed. The relative intensities of each probe were compared between the exofucosylated samples and the buffer-treated samples, analyzing them together (n=7), or in separate samples following either FTVI (n=4) or FTVII (n=3) treatments. No significant differences were detected for any of the comparisons, according to the Student's paired t test using the Benjamini-Hochberg correction for p value calculation. Therefore, the gene expression profile of HMSCs was not affected by exofucosylation.

FTVI- and FTVII-Mediated α(1,3)-Exofucosylation does not Affect Receptor Tyrosine Kinase Phosphorylation Profiles of hMSCs

We aimed to get insights into the potential changes in cell signaling induced by exofucosylation by analyzing the relative levels of phosphorylation of 71 different RTKs in lysates from hMSCs cultured in the presence of HPL and TrypLE Select reagent with or without exofucosylation. Comparing untreated, buffer-treated, FTVI-treated (n=7) and FTVII-treated (n=4) hMSCs there were no significant differences in RTK profiles according to the Student's paired t test, indicating that RTK phosphorylations in hMSCs are not affected by exofucosylation.

Results of Sepax Separation of Mononuclear Cells (MNCs) for Clinical Scale Validation of hMSC Cultures

Ficoll density gradient-based separation of bone marrow cells using the Sepax cell processor resulted in red blood cell and neutrophil depletion, with a median nucleated cell (NC) recovery of 23.7% (±12.9%) and an MNC (lymphocytes plus monocytes) recovery of 59.9% (±33.5%). We did not perform a CFU-fibroblast (CFU-F) assay to quantify nonhematopoietic fibroblastic colonies following Sepax separation, however, in all cases >10⁸ expanded BM-MSCs were obtained by the third passage. CPDs were less than 10 in all cases.

### Example 3—Discussion

In recent years, encouraging results have been published on the clinical safety and potential efficacy of hMSCs as a cell therapy medicinal product in diverse pathological entities. Preclinical studies and clinical trials, mostly phase-I and phase-II, have shown an absence of major adverse effects (40-44). Regarding efficacy, promising results of intravenous administration of autologous and allogeneic hMSCs have been obtained in pathologies such as osteogenesis imperfecta, refractory graft-versus-host disease, inflammatory bowel disease, and rheumatoid arthritis (17-21,45,46). However, the inability of systematically-administered cells to enter affected sites of tissue injury/inflammation is a factor that could be limiting the achievement of better clinical outcomes. Exofucosylation of hMSCs to enforce the CD44 glycoform HCELL, according to the results published by Sackstein et al. (25,47), increases the tropism of hMSCs for E-selectin-expressing tissues such as BM microvascular endothelium or inflamed tissue, and therefore is a highly promising approach that for MSC-based treatment of systemic diseases such as osteoporosis and autoimmune disease(s) (25,47). Accordingly, we aimed to develop and validate a consistent and reproducible manufacturing process for the production of clinical-grade exofucosylated hMSCs, with express intention of preserving, or optimally, augmenting the expression of terminal sialylated Type 2 lactosamines that could serve as substrates for exofucosylation thereby engendering expression of sLeX determinants and thereby programming binding to E-selectin.

Xenogeneic products used in typical in vitro experimentation, such as FBS and porcine trypsin, are not considered suitable for clinical-scale GMP cellular production. Therefore, our first goal was to determine whether exofucosylation of hMSCs expanded using HPL as culture supplement and TrypLE Select reagent to harness the plated cells was feasible. It has been reported that HPL, compared to FBS, improves the proliferative capacity of hMSCs and enhances their potential for osteogenic differentiation (48-51). These properties are of interest in relation to clinical application in osteoporosis, where native hMSCs in situ (within osteoporotic marrow) have been described to have reduced proliferative capacity and a higher trend towards adipogenic differentiation. There are no prior data about the in vitro safety profile of exofucosylated hMSCs expanded with HPL and manufactured according to GMP. In our validation study, the origin of 2 out of 3 BM samples were from osteoporotic patients aged 64 and 74; during the expansion phase of these samples using HPL, we observed expansion of >10⁸ hMSCs after just 2 and 3 passages, respectively Table II). These data indicate that hMSCs from osteoporotic marrow are capable of significant proliferation ex vivo.

Table II shows the culture and expansion of hMSCs before treatment with fucosyltransferases using HPL-containing medium. Primary culture of bone marrow mononuclear cells (BMMNCs) was performed and passaged to reach 100×10⁶ to 200×10⁶ of cells prior to exofucosylation, and cumulative population doublings (“duplications”) were assessed to confirm that HPL-supplementation would support sufficient expansion of cells. The first batch was obtained from a healthy donor (hMSCs-FUC-V/001), and the second batch represents cryopreserved cells of the first batch (RCB-hMSCs-FUC-V/001). Expansion of a cryopreserved sample was undertaken to assess whether banked cells could serve as back-up for a primary batch culture. The third and fourth batches (hMSCs-FUC-V/002 and hMSCs-FUC-V/003) were obtained from each of two osteoporotic donors. Note number of cells, passages, and duplications (cumulative population doublings). Cell viability was evaluated by Trypan Blue exclusion and more than 75% cell viability was consistently observed for all samples.

TABLE II

| LOT | PASSAGE | CELLS | DUPLICATIONS |
|---|---|---|---|
| BM-hMCSs-FUC-V/001 | 3 | 100 × 10⁶ | 9.9 |
| RCB-BM-hMSCs-FUC- | 3 | 100 × 10⁶ | 7.61 |
| V/001 | | | |
| BM-hMSCs-FUC-V/002 | 2 | 200 × 10⁶ | 4.4 |
| BM-hMSCs-FUC-V/003 | 3 | 130 × 10⁶ | 7.22 |

Our results also show that HPL does not negatively affect expression of hMSC lactosaminyl glycans, and, interestingly, higher levels of LeX (detected by both CSLEX-1 and HECA452 staining) were engendered using either FTVI or FTVII to exofucosylate the cells, and higher levels of CD15 (LeX) were engendered using FTVI to exofucosylate the cells, in hMSCs cultured under HPL conditions; these results indicate that HPL-supplementation increased expression of both sialylated and unsialylated Type 2 lactosamines on hMSCs compared to cells grown in FBS-supplementation. In addition, detachment of cells using TrypLE Select reagent, a recombinant protease, is suitable for use in GMP. This reagent is stable at RT, and thus simplifies management and storage requirements as compared to porcine trypsin which requires storage at −20° C. Furthermore, the exposure time needed to lift cells using this reagent is shorter, neutralization is not required, and cell viability is not affected by its use. Importantly, our data indicate that enzymatic detachment of hMSCs with TrypLE Select does not affect the efficiency of exofucosylation. Indeed, cell binding assays showed increased L-selectin-dependent lymphocyte adherence onto exofucosylated hMSCs expanded using HPL and lifted with TrypLE Select, and that HCELL expressed on these cells has functional similarities to that of native HCELL expressed on human hematopoietic cells.

To establish a robust, GMP-compliant, clinical-scale protocol to obtain large batches of exofucosylated hMSCs for clinical use, we sought to obtain at least 3 consecutive favorable results of expanded hMSCs that had undergone exofucosylation (52). Automated and closed cell isolation systems are currently being developed to ease clinical application of cell therapies, ensuring the reproducibility of the results. In our validation study, the Sepax cell processor was used to isolate bone marrow mononuclear cells. This system has been validated by other investigators using cord blood, peripheral blood, and bone marrow cells (53,54,55).

Three minimal criteria are conventionally used to identify hMSCs: (1) plastic adherence; (2) native expression of specific surface antigens (positivity for CD105, CD73 and CD90 (>90%)) and absence of expression (<5%-positivity) for CD45, CD34, CD14, CD11b, CD79a or CD19, and HLA class II); and (3) the ability to differentiate into osteoblasts, adipocytes and chondroblasts (56). In general, expansion should not exceed 4 passages or 20 CPDs in order to avoid genetic instability or cell senescence (57). Accordingly, our validation lots did not exceed 4 passages, and upon obtaining the desired cell number (1×10⁸), hMSCs were treated with fucosyltransferase (FTVII) in a physiological buffer fully comprised of GMP grade reagents. Exofucosylated hMSCs were then washed, conditioned, and packaged (final product). The manufacturing process validation was successfully completed in 4 consecutive batches, including one obtained from the replacement cell bank from the first donor. Notably, cell viability was not affected by FTVI or FTVII enzymatic treatments. Cell viability and expression of the engendered glycan (sLeX) was stable at 4° C. for at least 48 h. Neither the final product nor any of the culture phases of the validation lots suffered microbiological contamination, despite the withdrawal of antibiotics from the culture medium from passage 1 to avoid remaining traces in the final product.

To date, no associated tumorigenic risks have been reported in clinical trials using hMSCs (58-59). Exofucosylated CB-HSCs using FTVI have been already used in patients in BM transplant setting without any safety concern (60,61). In the process validation batches, genetic stability was evaluated by karyotypic analysis and tumorogenic potential by c-Myc mRNA expression levels. Karyotypes were normal in all the batches, which is consistent with previous studies (50,62). Our results support those by Crespo et al. (32) that report protection against chromosomal instability and maintenance of multipotentiality of hMSCs cultured with HPL as compared to FBS. hMSCs exhibited low levels of c-Myc expression, and these did not significantly change following treatment with FTVI or FTVII. The safety of exofucosylation is further supported by the observation that intrinsic cell signaling of hMSCs is unaltered, as assessed by analysis of the GEP and RTK profiles.

The investigational medicinal product based on expanded exofucosylated hMSCs is stable at 4° C. for 48 h. During this period, cell viability and immunophenotypic identity of hMSCs is preserved, and HCELL expression levels remain high, which allows cold shipping and facilitates the logistics of clinical applications. Collectively, our data indicate that culture methods can be chosen specifically such as to maintain or to augment expression of desired terminal Type 2 lactosamines, and thereby support the applicability of cell surface glycan engineering via exofucosylation to enforce HCELL expression on culture-expanded cells, such as hMSCs. The embodiments described herein thereby facilitate the implementation of clinical trials of stem cell therapy with ex vivo expanded exofucosylated hMSCs to program the migration of these cells to marrow, to skin, and to all inflammatory sites.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

### REFERENCES

The following documents, to the extent they provide exemplary procedural or other details supplementary to those set forth herein, are specifically incorporated herein by reference.
(1) Finger E B, Puri K D, Alon R, Lawrence M B, von Andrian U H, Springer T A. Adhesion through L-selectin requires a threshold hydrodynamic shear. Nature 1996; 379:266-9.
(2) Alon R, Chen S, Puri K D, Finger E B, Springer T A. The kinetics of L-selectin tethers and the mechanics of selectin-mediated rolling. J Cell Biol 1997; 138:1169-80.
(3) Butcher E C. Leukocyte-endothelial cell recognition: three (or more) steps to specificity and diversity. Cell 1991; 67:1033-6.
(4) Schreiber T H, Shinder V, Cain D W, Alon R, Sackstein R. Shear flow-dependent integration of apical and subendothelial chemokines in T-cell transmigration: implications for locomotion and the multistep paradigm. Blood 2007; 109:1381-6.
(5) Schweitzer K M, Drager A M, van der Valk P, Thijsen S F, Zevenbergen A, Theijsmeijer A P, et al. Constitutive expression of E-selectin and vascular cell adhesion molecule-1 on endothelial cells of hematopoietic tissues. Am J Pathol 1996; 148:165-75.
(6) Weninger W, Ulfman L H, Cheng G, Souchkova N, Quackenbush E J, Lowe J B, et al. Specialized contributions by alphα(1,3)-fucosyltransferase-IV and FucT-VII during leukocyte rolling in dermal microvessels. Immunity 2000; 12:665-76.
(7) Picker L J, Treer J R, Ferguson-Darnell B, Collins P A, Bergstresser P R, Terstappen L W. Control of lymphocyte recirculation in man. II. Differential regulation of the cutaneous lymphocyte-associated antigen, a tissue-selective homing receptor for skin-homing T cells. J Immunol Baltim Md 1950 1993; 150:1122-36.
(8) Vestweber D, Blanks J E. Mechanisms that regulate the function of the selectins and their ligands. Physiol Rev 1999; 79:181-213.
(9) Berg E L, Magnani J, Warnock R A, Robinson M K, Butcher E C. Comparison of L-selectin and E-selectin ligand specificities: the L-selectin can bind the E-selectin ligands sialyl Le(x) and sialyl Le(a). Biochem Biophys Res Commun 1992; 184:1048-55.
(10) Poppe L, Brown G S, Philo J S, Nikrad P V, Shah B H. Conformation of sLe^{X} Tetrasaccharide, Free in Solution and Bound to E-, P-, and L-Selectin, J Am Chem Soc 1997; 119:1727-36.
(11) Fuhlbrigge R C, Kieffer J D, Armerding D, Kupper T S. Cutaneous lymphocyte antigen is a specialized form of PSGL-1 expressed on skin-homing T cells. Nature 1997; 389:978-81.
(12) Dimitroff C J, Lee J Y, Rafii S, Fuhlbrigge R C, Sackstein R. CD44 is a major E-selectin ligand on human hematopoietic progenitor cells. J Cell Biol 2001; 153:1277-86.
(13) Fuhlbrigge R C, King S L, Sackstein R, Kupper T S. CD43 is a ligand for E-selectin on CLA+ human T cells. Blood 2006; 107:1421-6.
(14) Yang J, Furie B C, Furie B. The biology of P-selectin glycoprotein ligand-1: its role as a selectin counterreceptor in leukocyte-endothelial and leukocyte-platelet interaction. Thromb Haemost 1999; 81:1-7.
(15) Oxley S M, Sackstein R. Detection of an L-selectin ligand on a hematopoietic progenitor cell line. Blood 1994; 84:3299-306.
(16) Koc O N, Day J, Nieder M, Gerson S L, Lazarus H M, Krivit W. Allogeneic mesenchymal stem cell infusion for treatment of metachromatic leukodystrophy (MLD) and Hurler syndrome (MPS-IH). Bone Marrow Transplant 2002; 30:215-22.
(17) Moraleda J M, Blanquer M, Gomez-Espuch J, Iniesta F, Hurtado V, Perez-Espejo M A et al. Terapia con células madre en enfermedades neurodegenerativas. REVISTA DE HEMATOLOGÍA MEXICANA 2011; 12:144-14.
(18) Pérez-Simón J A, Lopez-Villar O, Andreu E J, Rifon J, Muntion S, Campelo M D, et al. Mesenchymal stem cells expanded in vitro with human serum for the treatment of acute and chronic graft-versus-host disease: results of a phase I/II clinical trial. Haematologica 2011; 7:1072-6.
(19) Garcia-Olmo D, Herreros D, Pascual I, Pascual J A, Del-Valle E, Zorrilla J, et al. Expanded adipose-derived stem cells for the treatment of complex perianal fistula: a phase II clinical trial. Dis Colon Rectum 2009; 52:79-86.
(20) Sanchez P L, Sánchez-Guijo F M, Villa A, del Callizo C, Arnold R, San Roman J A, et al. Launching a clinical program of stem cell therapy for cardiovascular repair. Nat Clin Pract Cardiovasc Med 2007; 4 (Suppl 1):5123-9.
(21) Le Blanc K. Immunomodulatory effects of fetal and adult mesenchymal stem cells. Cytotherapy 2003; 5:485-9.
(22) Friedenstein A J, Petrakova K V, Kurolesova A I, Frolova G P. Heterotopic of bone marrow. Analysis of precursor cells for osteogenic and hematopoietic tissues. Transplantation 1968; 6:230-47.
(23) Pittenger M F, Mackay A M, Beck S C, Jaiswal R K, Douglas R, Mosca J D, et al. Multilineage potential of adult human mesenchymal stem cells. Science 1999; 284:143-7.
(24) Pachón-Peña G, Donnelly C, Ruiz-Cañada C, Katz A, Fernández-Veledo S, Vendrell J. et al. A glycovariant of human CD44 is characteristically expressed on human mesenchymal stem cells. Stem Cells 2017; 35:1080-92.
(25) Sackstein R, Merzaban J S, Cain D W, Dagia N M, Spencer J A, Lin C P, et al. Ex vivo glycan engineering of CD44 programs human multipotent mesenchymal stromal cell trafficking to bone. Nat Med 2008; 14:181-7.
(26) Sather B D, Treuting P, Perdue N, Miazgowicz M, Fontenot J D, Rudensky A Y, et al. Altering the distribution of Foxp3(+) regulatory T cells results in tissue-specific inflammatory disease. J Exp Med 2007; 204:1335-47. doi:10.1084/jem.20070081.
(27) Sensebé L, Bourin P, Tarte K. Good manufacturing practices production of mesenchymal stem/stromal cells. Hum Gene Ther 2011; 22:19-26.
(28) Reinhardt J, Stühler A, Blúmel J. Safety of bovine sera for production of mesenchymal stem cells for therapeutic use. Hum Gene Ther 2011; 22:775; author reply 776.
(29) Kinzebach S, Bieback K. Expansion of Mesenchymal Stem/Stromal cells under xenogenic-free culture conditions. Adv Biochem Eng Biotechnol 2013; 129:33-57.
(30) Schallmoser K, Bartmann C, Rohde E, Reinisch A, Kashofer K, Stadelmeyer E, et al. Human platelet lysate can replace fetal bovine serum for clinical-scale expansion of functional mesenchymal stromal cells. Transfusion (Paris) 2007; 47:1436-46.
(31) Fekete N, Gadelorge M, Fúrst D, Maurer C, Dausend J, Fleury-Cappellesso S, et al. Platelet lysate from whole blood-derived pooled platelet concentrates and apheresis-derived platelet concentrates for the isolation and expansion of human bone marrow mesenchymal stromal cells: production process, content and identification of active components. Cytotherapy 2012; 14:540-54.
(32) Crespo-Díaz R, Behfar A, Butler G W, Padley D J, San M G, Bartunek J, et al. Platelet lysate consisting of a natural repair proteome supports human mesenchymal stem cell proliferation and chromosomal stability. Cell Transplant 2011; 20:797-811.
(33) Dahl J-A, Duggal S, Coulston N, Millar D, Melki J, Shandadfar A, et al. Genetic and epigenetic instability of human bone marrow mesenchymal stem cells expanded in autologous serum or fetal bovine serum. Int J Dev Biol 2008; 52:1033-42.
(34) Dreher L, Elvers-Hornung S, Brinkmann I, Huck V, Henschler R, Gloe T, et al. Cultivation in human serum reduces adipose tissue-derived mesenchymal stromal cell adhesion to laminin and endothelium and reduces capillary entrapment. Stem Cells Dev 2013; 22:791-803.
(35) Furlani D, Ugurlucan M, Ong L, Bieback K, Pittermann E, Westien I, et al. Is the intravascular administration of mesenchymal stem cells safe? Mesenchymal stem cells and intravital microscopy. Microvasc Res 2009; 77:370-6.
(36) Ramot Y, Steiner M, Morad V, Leibovitch S, Amouyal N, Cesta M F, et al. Pulmonary thrombosis in the mouse following intravenous administration of quantum dot-labeled mesenchymal cells. Nanotoxicology 2010; 4:98-105.
(37) Dominici M, Le Blanc K, Mueller I, Slaper-Cortenbach I, Marini F, Krause D, et al. Minimal criteria for defining multipotent mesenchymal stromal cells. The International Society for Cellular Therapy position statement. Cytotherapy 2006; 8:315-7.
(38) Reyes B, Coca M I, Codinach M, Lopez-Lucas M D, Mazo-Barbara A, Caminal M, et al. Assessment for study design and challenges in detection methodologies. Cytotherapy 2017; 19:1060-69.
(39) Blanquer M, Pérez Espejo M A, Iniesta F, Gómez Espuch J, Meca J, Villaverde R, et al. Bone marrow stem cell transplantation in amyotrophic lateral sclerosis: technical aspects and preliminary results from a clinical trial. Methods Find Exp Clin Pharmacol 2010; 32 Suppl A:31-7.
(40) Packham D K, Fraser I R, Kerr P G, Segal K R. Allogeneic Mesenchymal Precursor Cells (MPC) in Diabetic nephropathy: a randomized, placebo-controlled, dose escalation study. EBioMedicine 2016; 263-9.
(41) Stolk J, Broekman W, Mauad T, et al. A phase I study for intravenous autologous mesenchymal stromal cell administration to patients with severe emphysema. QJM 2016; 109:331-6.
(42) Saad A, Dietz A B, Herrmann S M S, Hickson U, Glockner J F, McKusick M A, et al. Autologous mesenchymal stem cells increase cortical perfusion in renovascular disease. J Am Soc Nephrol 2017; 28:2777-85.
(43) Wang D, Zhang H, Liang J, Wang H, Hua B, Feng X, Gilkeson G S, Farge D, Shi S, Sun L. A Long-Term Follow-Up Study of Allogeneic Mesenchymal Stem/Stromal Cell Transplantation in Patients with Drug-Resistant Systemic Lupus Erythematosus. Stem Cell Reports. 2018 Mar. 13; 10(3):933-941. doi: 10.1016/j.stemcr.2018.01.029. Epub 2018 Mar. 1.
(44) Xu, J., Wang, D., Liu, D., Fan, Z., Zhang, H., Liu, O., Ding, G., Gao, R., Zhang, C., Ding, Y., et al. (2012). Allogeneic mesenchymal stem cell treatment alleviates experimental and clinical Sjogren's syndrome. Blood 120, 3142-3151. 45) Bueno C, Ramirez C, Rodríguez-Lozano F J, Tabarés-Seisdedos R, Rodenas M, Moraleda J M, et al. Human adult periodontal ligament-derived cells integrate and differentiate after implantation into the adult mammalian brain. Cell Transplant 2013; 22:2017-28.
(45) Toonkel R L, Hare J M, Matthay M A, Glassberg M K. Mesenchymal stem cells and idiopathic pulmonary fibrosis. Potential for clinical testing. Am J Respir Crit Care Med 2013; 188:133-40.
(46) Pastor D, Viso-Léon M C, Botella-López A, Jaramillo-Merchan J, Moraleda J M, Jones J, et al. Bone marrow transplantation in hindlimb muscles of motoneuron degenerative mice reduces neuronal death and improves motor function. Stem Cells Dev 2013; 22:1633-44.
(47) Thankamony S P, Sackstein R. Enforced hematopoietic cell E- and L-selectin ligand (HCELL) expression primes transendothelial migration of human mesenchymal stem cells. Proc Natl Acad Sci USA 2011; 108:2258-63.
(48) Bieback K, Hecker A, Kocaomer A, Lannert H, Schallmoser K, Strunk D, et al. Human alternatives to fetal bovine serum for the expansion of mesenchymal stromal cells from bone marrow. Stem Cells Dayt Ohio 2009; 27:2331-41.
(49) Bernardo M E, Avanzini M A, Perotti C, Cometa A M, Moretta A, Lenta E, et al. Optimization of in vitro expansion of human multipotent mesenchymal stromal cells for cell-therapy approaches: further insights in the search for a fetal calf serum substitute. J Cell Physiol 2007; 211:121-30.
(50) Doucet C, Ernou I, Zhang Y, Llense J-R, Begot L, Holy X, et al. Platelet lysates promote mesenchymal stem cell expansion: a safety substitute for animal serum in cell-based therapy applications. J Cell Physiol 2005; 205:228-36.
(51) Chevallier N, Anagnostou F, Zilber S, Bodivit G, Maurin S, Barrault A, et al. Osteoblastic differentiation of human mesenchymal stem cells with platelet lysate. Biomaterials 2010; 31:270-8.
(52) EudraLex. Volume 4: EU Guidelines to Good Manufacturing Practice Medicinal Products for Human and Veterinary Use. Annex 2: Manufacture of Biological active substances and Medicinal Products for Human Use (2013). Directives 91/356/EEC, as amended by Directive 2003/94/E C, and 91/412/EEC n.d.
(53) Giiven S, Karagianni M, Schwalbe M, Schreiner S, Farhadi J, Bula S, et al. Validation of an automated procedure to isolate human adipose tissue-derived cells by using the Sepax® technology. Tissue Eng Part C Methods 2012; 18:575-82.
(54) Aktas M, Radke T F, Strauer B E, Wernet P, Kogler G. Separation of adult bone marrow mononuclear cells using the automated closed separation system Sepax. Cytotherapy 2008; 10:203-11.
(55) Zinno F, Landi F, Scerpa M C, Aureli V, Lanti A, Ceccarelli S, et al. Processing of hematopoietic stem cells from peripheral blood before cryopreservation: use of a closed automated system. Transfusion (Paris) 2011; 51:2656-63.
(56) Jones E A, English A, Henshaw K, Kinsey S E, Markham A F, Emery P, et al. Enumeration and phenotypic characterization of synovial fluid multipotential mesenchymal progenitor cells in inflammatory and degenerative arthritis. Arthritis Rheum 2004; 50:817-27.
(57) Bertolo A, Mehr M, Janner-Jametti T, Graumann U, Aebli N, Baur M, et al. An in vitro expansion score for tissue-engineering applications with human bone marrow-derived mesenchymal stem cells. J Tissue Eng Regen Med 2013; 10:149-61.
(58) Wagner W, Wein F, Seckinger A, Frankhauser M, Wirkner U, Krause U, et al. Comparative characteristics of mesenchymal stem cells from human bone marrow, adipose tissue, and umbilical cord blood. Exp Hematol 2005; 33:1402-16.
(59) Ishii M, Koike C, Igarashi A, Yamanaka K, Pan H, Higashi Y, et al. Molecular markers distinguish bone marrow mesenchymal stem cells from fibroblasts. Biochem Biophys Res Commun 2005; 332:297-303.
(60) Jones E, McGonagle D. Human bone marrow mesenchymal stem cells in vivo. Rheumatol Oxf Engl 2008; 47:126-31.
(61) Popat U, Mehta R S, Rezvani K, Fox P, Kondo K, Marin D, et al. Enforced fucosylation of cord blood hematopoietic cells accelerates neutrophil and platelet engraftment after transplantation. Blood 2015; 125:2885-92.
(62) Zhang Z-X, Guan L-X, Zhang K, Wang S, Cao P-C, Wang Y-H, et al. Cytogenetic analysis of human bone marrow-derived mesenchymal stem cells passaged in vitro. Cell Biol Int 2007; 31:645-8.
(63) Sackstein, R. (2009) Glycosyltransferase-programmed stereosubstitution (GPS) to create HCELL: engineering a roadmap for cell migration. Immunol Rev 230, 51-74
(64) Sackstein, R. (2005) The lymphocyte homing receptors: gatekeepers of the multistep paradigm. Curr. Opin. Hematol. 12(6): 444-50
(65) Lapidot, T., Dar, A., Kollet, O., (2005) How do stem cells find their way home? Blood 106(6): 1901-10
(66) Springer, T. A., (1994) Traffic signals for lymphocyte recirculation and leukocyte emigration: the multistep paradigm. Cell 76(2):301-14
(67) Peled, A., Petit, I., Kollet, O., Magid, M., Ponomaryov, T., Byk, T., Nagler, A., Ben-Hur, H., Many, A., Shultz, L., Lider, O., Alon, R., Zipori, D., Lapidot, T., (1999) Science 283(5403):845-8
(68) Sipkins, D. A., Wei, X., Wu, J. W., Runnels, J. M., Côté, D., Means, T. K., Luster, A. D., Scadden, D. T., Lin, C. P., (2005) Nature 435(7044):969-73
(69) Sackstein, R., (2009) Glycosyltransferase-programmed stereosubstitution (GPS) to create HCELL: engineering a roadmap for cell migration. Immunol. Rev. 230(1):51-74
(70) Polley, M. J., Phillips, M. L., Wayner, E., Nudelman, E., Singhal, A. K., Hakomori, S., Paulson, J. C., (1991) CD62 and endothelial cell-leukocyte adhesion molecule 1 (ELAM-1) recognize the same carbohydrate ligand, sialyl-Lewis x. Proc Natl Acad Sci USA 88(14):6224-8
(71) Laszik, Z., Jansen, P. J., Cummings, R. D., Tedder, T. F., McEver, R. P., Moore, K. L., (1996) P-selectin glycoprotein ligand-1 is broadly expressed in cells of myeloid, lymphoid, and dendritic lineage and in some nonhematopoietic cells. Blood 88(8):3010-21
(72) Dimitroff, C. J., Lee, J. Y., Rafii, S., Fuhlbrigge, R. C., Sackstein, R., (2001) CD44 is a major E-selectin ligand on human hematopoietic progenitor cells. J Cell Biol 153(6): 1277-86
(73) Dimitroff, C. J., Lee, J. Y., Fuhlbrigge, R. C., Sackstein, R., (2000) A distinct glycoform of CD44 is an L-selectin ligand on human hematopoietic cells. Proc Natl Acad Sci USA 97(25): 13841-6
(74) Fuhlbrigge, R. C., King, S. L., Sackstein, R., Kupper, T. S. (2006) CD43 is a ligand for E-selectin on CLA+ human T cells. Blood 107(4):1421-6
(75) Sackstein, R., Dimitroff, C. J., (2000) A hematopoietic cell L-selectin ligand that is distinct from PSGL-1 and displays N-glycan-dependent binding activity. Blood 96(8):2765-74
(76) Dimitroff, C. J., Lee, J. Y., Schor, K. S., Sandmaier, B. M., Sackstein, R., (2001) differential L-selectin binding activities of human hematopoietic cell L-selectin ligands, HCELL and PSGL-1. J Biol Chem 276(50):47623-31
(77) Dimitroff, C. J., Lee, J. Y., Rafii, S., Fuhlbrigge, R. C., Sackstein, R., (2001) CD44 is a major E-selectin ligand on human hematopoietic progenitor cells. J Cell Biol 153(6): 1277-86
(78) Merzaban, J. S., Burdick, M. M., Gadhoum, S. Z., Dagia, N. M., Chu, J. T., Fuhlbrigge, R. C., and Sackstein, R. (2011) Analysis of glycoprotein E-selectin ligands on human and mouse marrow cells enriched for hematopoietic stem/progenitor cells. Blood 118, 1774-1783
(79) Mondal, N., Dykstra, B., Lee, J., Ashline, D. J., Reinhold, V. N., Rossi, D. J., Sackstein, R., Distinct human α(1,3)-fucosyltransferases drive Lewis-X/sialyl Lewis-X assembly in human cells. (2018) J. Biol. Chem. 293(19) 7300-7314
(80) Dykstra, B., Lee, J., Mortensen, L. J., Yu, H., Wu, Z. L., Lin, C. P., Rossi, D. J., and Sackstein, R. (2016) Glycoengineering of E-Selectin Ligands by Intracellular versus Extracellular Fucosylation Differentially Affects Osteotropism of Human Mesenchymal Stem Cells. Stem Cells 34, 2501-2511
(81) Sackstein, R., Merzaban, J. S., Cain, D. W., Dagia, N. M., Spencer, J. A., Lin, C. P., and Wohlgemuth, R. (2008) Ex vivo glycan engineering of CD44 programs human multipotent mesenchymal stromal cell trafficking to bone. Nat Med 14, 181-187
(82) Gooi, H. C., Feizi, T., Kapadia, A., Knowles, B. B., Solter, D., and Evans, M. J. (1981) Stage-specific embryonic antigen involves alpha 1 goes to 3 fucosylated type 2 blood group chains. Nature 292, 156-158
(83) Solter, D., and Knowles, B. B. (1978) Monoclonal antibody defining a stage-specific mouse embryonic antigen (SSEA-1). Proc Natl Acad Sci USA 75, 5565-5569
(85) Capela, A., and Temple, S. (2002) LeX/ssea-1 is expressed by adult mouse CNS stem cells, identifying them as nonependymal. Neuron 35, 865-875
(86) Klassen, H., Schwartz, M. R., Bailey, A. H., and Young, M. J. (2001) Surface markers expressed by multipotent human and mouse neural progenitor cells include tetraspanins and non-protein epitopes. Neurosci Lett 312, 180-182
(87) Yanagisawa, M., Taga, T., Nakamura, K., Ariga, T., and Yu, R. K. (2005) Characterization of glycoconjugate antigens in mouse embryonic neural precursor cells. J Neurochem 95, 1311-1320
(88) Pruszak, J., Ludwig, W., Blak, A., Alavian, K., and Isacson, O. (2009) CD15, CD24, and CD29 Define a Surface Biomarker Code for Neural Lineage Differentiation of Stem Cells. STEM CELLS 27, 2928-2940
(89) Yagi, H., Saito, T., Yanagisawa, M., Yu, R. K., and Kato, K. (2012) Lewis X-carrying N-glycans regulate the proliferation of mouse embryonic neural stem cells via the Notch signaling pathway. J Biol Chem 287, 24356-24364
(90) van Gisbergen, K. P., Sanchez-Hernandez, M., Geijtenbeek, T. B., and van Kooyk, Y. (2005) Neutrophils mediate immune modulation of dendritic cells through glycosylation-dependent interactions between Mac-1 and DC-SIGN. J Exp Med 201, 1281-1292

What is claimed is:

1. A method of detecting a change of expression in cell-surface Type 2 terminal lactosamines on a population of cultured cells comprising the steps of:
(a) contacting the cells with a glycosyltransferase and a donor nucleotide sugar, wherein the glycosyltransferase and donor nucleotide sugar are effective to enforce expression of a glycan; and
(b) detecting the product glycan on the cells,
wherein the contacting of step (a) is performed before and/or after any culture condition modification.

2. The method of claim 1, wherein the detecting of step (b) comprises an antibody-based technique that recognizes the product glycan.

3. The method of claim 1, wherein the detecting of step (b) is effective to precisely identify the Type 2 terminal lactosamine target of an α(1,3)-fucosyltransferase by detecting one or more of product glycans consisting of sLeX, LeX, VIM-2, and Difucosyl sLeX.

4. A method of detecting differences in level of expression of cell-surface Type 2 terminal lactosamines on a population of cultured cells propagated under different conditions comprising the steps of:
(a) culturing a first population of cells under a first culture condition;
(b) culturing a second population of cells under a second (different) culture condition;
(c) contacting the first and second population of cells with a glycosyltransferase and a donor nucleotide sugar, wherein the glycosyltransferase and donor nucleotide sugar are effective to enforce expression of a glycan; and
(d) detecting the glycan on the first and second population of cells.

5. The method of claim 4, wherein the glycosyltransferase is an α(1,3)-fucosyltransferase, and the detection step (d) is effective to precisely identify the Type 2 terminal lactosamine target of the fucosyltransferase by detecting one or more of glycans consisting of sLeX, LeX, VIM-2, and Difucosyl sLeX.

6. The method of claim 4, wherein the first and second culture conditions comprise different supplements.

7. The method of claim 4, wherein the first and second population of cells are frozen and then thawed after the contacting with the glycosyltransferase of step (c).

8. The method of claim 4, further comprising the step of (e) selecting the culture condition that is effective to produce a desired amount of Type 2 lactosaminyl glycan.

9. The method of claim 4, wherein the glycosyltransferase is α(1,3)-fucosyltransferase VI, α(1,3)-fucosyltransferase VII, or a combination thereof.

10. A process for producing GMP-grade exofucosylated cells comprising:
(a) providing cells with a culture medium comprising a supplement, wherein the cells comprise cell surface CD44 and the supplement is effective to maintain or increase the amount of a CD44 glycoform comprising sialylated Type 2 lactosamines;
(b) expanding the cells in the culture medium; and
(c) contacting the cells with a glycosyltransferase and a donor nucleotide sugar that are effective to enforce expression of the HCELL glycoform of CD44 on the cells.

11. The process of claim 10, further comprising the step of storing the cells at 4° C. or less;
wherein an HCELL glycoform of CD44 is stably expressed and cell viability is maintained for at least 48 hours.

12. The process of claim 10, further comprising (d) freezing and then thawing the cells, wherein the cells stably express the HCELL glycoform of CD44 after thawing.

13. The process of claim 10, wherein the glycotransferase is selected from the group consisting of α(1,3)-fucosyltransferase III, IV, V, VI, VII, IX or a combination thereof.

14. The system of claim 13, wherein the glycosyltransferase is effective to enforce the HCELL glycoform of CD44 on the cells.

15. The system of claim 13, wherein the cells are human mesenchymal stem cells (hMSCs).

16. The system of claim 10, wherein the supplement is human platelet lysate (HPL).

17. The system of claim 14, where the cells stably express the HCELL glycoform of CD44 on the hMSCs after freezing and then thawing the cells.

18. The method of claim 6, further comprising the step of selecting a media supplement that is effective to maintain or increase the amount of a sialylated Type 2 lactosamine on a cell.
